SEQUENCE LISTING (72 sequences)

SEQ ID NO: 1 - protein, 10 aa, Homo sapiens
LLSLGAGEFK

SEQ ID NO: 2 - protein, 10 aa, Homo sapiens
LLSLGAGEFR

SEQ ID NO: 3 - protein, 11 aa, Homo sapiens
LPDATPTELAK

SEQ ID NO: 4 - protein, 11 aa, Homo sapiens
LPEATPTELAK

SEQ ID NO: 5 - protein, 13 aa, Homo sapiens
AADDTWEPFSSGK

SEQ ID NO: 6 - protein, 13 aa, Homo sapiens
AADDTWEPFASVK

SEQ ID NO: 7 - protein, 11 aa, Homo sapiens
LGADMEDVCGR

SEQ ID NO: 8 - protein, 9 aa, Homo sapiens
LGADMEDVR

SEQ ID NO: 9 - protein, 9 aa, Homo sapiens
LAVYQAGAR

SEQ ID NO: 10 - protein, 10 aa, Homo sapiens
CLAVYQAGAR

SEQ ID NO: 11 - protein, 9 aa, Homo sapiens
EHSSLAFWK

SEQ ID NO: 12 - protein, 9 aa, Homo sapiens
EHSSLAFSK

SEQ ID NO: 13 - protein, 8 aa, Homo sapiens
LTVSIEAR

SEQ ID NO: 14 - protein, 8 aa, Homo sapiens
LTMSIEAR

SEQ ID NO: 15 - protein, 9 aa, Homo sapiens
QTALVELVK

SEQ ID NO: 16 - protein, 9 aa, Homo sapiens
QTALVELMK

SEQ ID NO: 17 - protein, 12 aa, Homo sapiens
AVMDDFAAFVEK

SEQ ID NO: 18 - protein, 12 aa, Homo sapiens
AVMDAFAAFVEK

SEQ ID NO: 19 - protein, 12 aa, Homo sapiens
AVMDGFAAFVEK

SEQ ID NO: 20 - protein, 19 aa, Homo sapiens
SDNCEDTPEAGYFAIAVVK

SEQ ID NO: 21 - protein, 19 aa, Homo sapiens
SDNCEDTPEAGYFAVAVVK

SEQ ID NO: 22 - protein, 12 aa, Homo sapiens
PVEEYANCHLAR

SEQ ID NO: 23 - protein, 12 aa, Homo sapiens
SVEEYANCHLAR

SEQ ID NO: 24 - protein, 9 aa, Homo sapiens
WDRELISER

SEQ ID NO: 25 - protein, 9 aa, Homo sapiens
WDWELISER

SEQ ID NO: 26 - protein, 38 aa, Homo sapiens
VVPDSTPSWANPSPTPVISMLAQGSQVLESTPPPHVMR

SEQ ID NO: 27 - protein, 38 aa, Homo sapiens
VVPDSTPSWANPSATPVISMLAQGSQVLESTPPPHVMR

SEQ ID NO: 28 - protein, 56 aa, Homo sapiens
ADTHDEILEGLNFNLTEIPEAQIHEGFQELLRTLNQPDSQLQLTTGNGLFLSEGLK

SEQ ID NO: 29 - protein, 56 aa, Homo sapiens
ADTHDEILEGLNFNLTEIPEAQIHEGFQELLHTLNQPDSQLQLTTGNGLFLSEGLK

SEQ ID NO: 30 - protein, 16 aa, Homo sapiens
DTEEEDFHVDQVTTVK

SEQ ID NO: 31 - protein, 16 aa, Homo sapiens
DTEEEDFHVDQATTVK

SEQ ID NO: 32 - protein, 12 aa, Homo sapiens
PFVFLMIEQNTK

SEQ ID NO: 33 - protein, 12 aa, Homo sapiens
PFVFLMIDQNTK

SEQ ID NO: 34 - protein, 7 aa, Homo sapiens
GSWYSMR

SEQ ID NO: 35 - protein, 7 aa, Homo sapiens
GSWYSMK

SEQ ID NO: 36 - protein, 12 aa, Homo sapiens
YLQEIYNSNNQK

SEQ ID NO: 37 - protein, 12 aa, Homo sapiens
YLQEIHNSNNQK

SEQ ID NO: 38 - protein, 10 aa, Homo sapiens
VTSIQDWVQK

SEQ ID NO: 39 - protein, 10 aa, Homo sapiens
VTSIQHWVQK

SEQ ID NO: 40 - protein, 31 aa, Homo sapiens
PGSSGPGSTGSWNSGSSGTGSTGNQNPGSPR

SEQ ID NO: 41 - protein, 31 aa, Homo sapiens
PGSSGPGSAGSWNSGSSGTGSTGNQNPGSPR

SEQ ID NO: 42 - protein, 11 aa, Homo sapiens
LPAATPTELAK

SEQ ID NO: 43 - protein, 15 aa, Homo sapiens
QNQCFYNSSYLNVQR

SEQ ID NO: 44 - protein, 15 aa, Homo sapiens
QNQCFYNSSYLNAQR

SEQ ID NO: 45 - protein, 21 aa, Homo sapiens
LAILPASAPPATSNPDPAVSR

SEQ ID NO: 46 - protein, 21 aa, Homo sapiens
LAILPASATPATSNPDPAVSR

SEQ ID NO: 47 - protein, 24 aa, Homo sapiens
LLLLCLAGLVFVSEAGPTGTGESK

SEQ ID NO: 48 - protein, 24 aa, Homo sapiens
LLLLCLAGLVFVSEAGPTGTSESK

SEQ ID NO: 49 - protein, 26 aa, Homo sapiens
ANLSSQALQMSLDYGFVTPLTSMSIR

SEQ ID NO: 50 - protein, 26 aa, Homo sapiens
ANLSSQALRMSLDYGFVTPLTSMSIR

SEQ ID NO: 51 - protein, 22 aa, Homo sapiens
PLANVTLTCQAHLETPDFQLFK

SEQ ID NO: 52 - protein, 22 aa, Homo sapiens
PLANVTLTCQARLETPDFQLFK

SEQ ID NO: 53 - protein, 15 aa, Homo sapiens
DELTKNQVSLTCLVK

SEQ ID NO: 54 - protein, 15 aa, Homo sapiens
EELTKNQVSLTCLVK

SEQ ID NO: 55 - protein, 15 aa, Homo sapiens
DELTKNQVSLTCLVK

SEQ ID NO: 56 - protein, 15 aa, Homo sapiens
DEMTKNQVSLTCLVK

SEQ ID NO: 57 - protein, 23 aa, Homo sapiens
WQQGNVFSCSVMHEALHNHYTQK

SEQ ID NO: 58 - protein, 23 aa, Homo sapiens
WQQGNVFSCSVMHEGLHNHYTQK

SEQ ID NO: 59 - protein, 22 aa, Homo sapiens
GFYPSDISVEWESNGQPENNYK

SEQ ID NO: 60 - protein, 22 aa, Homo sapiens
GFYPADISVEWESNGQPENNYK

SEQ ID NO: 61 - protein, 58 aa, Homo sapiens
DYFPEPVTVSWNSGALTSGVHTFPAVLQSSGLYSLSSVVTVPSSNFGTQTYTCNVDHK

SEQ ID NO: 62 - protein, 58 aa, Homo sapiens
DYFPEPVTVSWNSGALTSGVHTFPAVLQSSGLYSLSSVVTVTSSNFGTQTYTCNVDHK

SEQ ID NO: 63 - protein, 33 aa, Homo sapiens
TPEVTCVVVDVSHEDPEVQFNWYVDGVEVHNAK

SEQ ID NO: 64 - protein, 33 aa, Homo sapiens
TPEVTCVVVDVSHEDPEVQFNWYVDGMEVHNAK

SEQ ID NO: 65 - protein, 9 aa, Homo sapiens
SAVQGPPER

SEQ ID NO: 66 - protein, 9 aa, Homo sapiens
SAVQGPPDR

SEQ ID NO: 67 - protein, 21 aa, Homo sapiens
GLTFQQNASSMCVPDQDTAIR

SEQ ID NO: 68 - protein, 21 aa, Homo sapiens
GLTFQQNASSMCGPDQDTAIR

SEQ ID NO: 69 - protein, 13 aa, Homo sapiens
ESDWLGQSMFTCR

SEQ ID NO: 70 - protein, 13 aa, Homo sapiens
ESDWLSQSMFTCR

SEQ ID NO: 71 - protein, 21 aa, Homo sapiens
VYACEVTHQGLSSPVTKSFNR

SEQ ID NO: 72 - protein, 21 aa, Homo sapiens
LYACEVTHQGLSSPVTKSFNR

(19) United States
(12) Patent Application Publication
(10) Pub. No.: US 2025/0102522 A1
(43) Pub. Date: Mar. 27, 2025

(54) METHODS OF TRACKING DONOR CELLS IN A RECIPIENT

(71) Applicant: Hepatx Corporation, Palo Alto, CA (US)

(72) Inventors: Eric R. SCHUUR, Palo Alto, CA (US); Marc CHAO, Palo Alto, CA (US); Pier Federico GHERARDINI, Palo Alto, CA (US); Philip E. GEYER, Planegg (DE); Sebastian Virreira WINTER, Planegg (DE); Johannes B. MÜLLER-REIF, Planegg (DE); Sophia DOLL, Planegg (DE)

(21) Appl. No.: 18/698,329

(22) PCT Filed: Oct. 6, 2022
(86) PCT No.: PCT/US2022/077688

§ 371 Date: Apr. 3, 2024

Related U.S. Application Data
(60) Provisional application No. 63/262,243, filed on Oct. 7, 2021.

(51) Int. Cl. G01N 33/68

(52) U.S. Cl. CPC G01N 33/6893; G01N 33/6848; G01N 2333/976; G01N 2800/52

(57) ABSTRACT

The present invention provides methods for detecting and/or monitoring the function a donor cell in a recipient by detecting specific isoforms of one or more polypeptides in the sample; wherein the donor cell and the recipient express different specific isoforms of each of the one or more polypeptides. In some embodiments, the specific isoforms are detected by mass spectrometry. The invention also provides methods of treatment with donor cells where the donor cells are monitored by the methods described herein.

### CROSS REFERENCE TO RELATED APPLICATIONS

This application claims the priority benefit of Provisional Patent Application No. 63/262,243, filed on Oct. 7, 2021, the content of which is incorporated herein by reference in its entirety.

### REFERENCE TO AN ELECTRONIC SEQUENCE LISTING

The content of the electronic sequence listing (776152000240seqlist.xml; Size: 63,435 bytes; and Date of Creation: Oct. 4, 2022) is herein incorporated by reference in its entirety.

### FIELD OF THE INVENTION

The present invention relates to methods for detecting and monitoring donor cells in a recipient.

### BACKGROUND OF THE INVENTION

The development of technologies for manufacturing different cell types in vitro has paved the way for the development of regenerative medicine both for tissue repair and as an alternative to organ transplantation, or for indications where transplants are difficult. The basic approach involves producing a specific cell type of the organ of interest in vitro, which is then administered to the patient to regenerate the organ whose function has been compromised by disease or trauma.

This paradigm has been applied to several organs with different degrees of success. Clinical improvement has been observed with this approach, however these improvements are often transient. One of the main problems in the interpretation of these clinical trial data is tracking of the transplanted cells in vivo. To justify observed clinical improvements in the absence of the ability to detect cell engraftment, investigators resorted to claiming that the administered cells acted by secreting some unspecified paracrine factors, which promoted tissue regeneration or modulated immune function. However, there has been no way to validate such claims directly, hampering the establishment of a solid causal link between intervention and clinical outcome.

While many labeling approaches for tracking transplanted cells in vivo can be used in research settings, only two labeling methods have been used in humans: (i) PET imaging of radionuclides, and (ii) MRI imaging using superparamagnetic iron oxide nanoparticles (SPIO) (Naumova, A et al., 2014, Nature Biotechnology 32:804-818). Both methods require labeling of the injected cells with a contrast agent before infusion, followed by in-vivo imaging. Radionuclides have half-lives that range from hours to a few days, which limit the utility of PET imaging. SPIO enables tracking for several weeks and this method has been successfully used in several clinical settings (Bulte, J W, 2009, AJR Am J Roentgenol 193 (2): 314-25.; Cromer Berman S M et al., 2011, Wiley Interdiscip Rev Nanomed Nanobiotechnol 3 (4): 343-55). Although this approach is promising, it has significant limitations: (i) the label is diluted with each cell division, which reduces signal intensity; (ii) SPIO imaging cannot distinguish between live and dead cells, and provides no information about the function of the transplanted cells; (iii) the debris from dead cells is scavenged by macrophages, which can result in a false positive signal; (iv) hypo-intense areas (e.g. hemorrhage, blood clot) give a signal that is similar to SPIO, which is another source of false positive signals; and (iv) the label can affect important properties of stem cells (Cromer Berman et al., 2013, Magn Reson Med 69 (1): 255-62).

### BRIEF SUMMARY

In some aspects, the invention provides a method for detecting a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, the invention provides a method for detecting a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, the invention provides a method for quantitating a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates a quantitative measure of the donor cells compared to the recipient cells or a measure of the performance of the donor cells in the recipient. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor cell in the recipient. In some embodiments, the method further comprises obtaining the amount of the donor-specific isoforms of the one or more polypeptides, wherein the amount of the donor-specific isoforms of the one or more polypeptides reflects the quantity of the donor cell in the recipient.

In some aspects, the invention provides a method for quantitating a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates a quantitative measure of the donor cells compared to the recipient cells or a measure of the performance of the donor cells in the recipient. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor cell in the recipient. In some embodiments, the method further comprises obtaining the amount of the donor-specific isoforms of the one or more polypeptides, wherein the amount of the donor-specific isoforms of the one or more polypeptides reflects the quantity of the donor cell in the recipient.

In some aspects, the invention provides a method for monitoring engraftment and/or function of a donor cell in a recipient, the method comprising a) isolating samples from the recipient one or more times after administration of the donor cell to the recipient; and b) detecting specific isoforms of one or more polypeptides in the samples; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of donor cells in the recipient.

In some aspects, the invention provides a method for monitoring engraftment and/or function of a donor cell in a recipient, the method comprising detecting specific isoforms of one or more polypeptides in samples from the recipient; wherein the samples have been obtained from the recipient one or more times after administration of the donor cell to the recipient; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of donor cells in the recipient.

In some embodiments according to any of the methods described above, the one or more samples are isolated or obtained from the recipient within at least about one week, such as within about two weeks, about three weeks, about one month, about two months, about three months, about four months, about five months, about six months, about nine months, about one year, or more than one year after administration of the donor cells.

In some embodiments according to any of the methods described above, the sample is a plasma sample, a serum sample, a whole blood sample, a lymph sample, a cerebrospinal fluid sample, a urine sample or a liquid or solid biopsy sample.

In some embodiments according to any of the methods described above, the donor cells are stem cells. In some embodiments, the stem cells are adult stem cells. In some embodiments, the adult stem cells are stromal stem cells, optionally, mesenchymal stromal stem cells, or stem cells derived from tissue, optionally adipose tissue, bone marrow, umbilical cord tissue, liver, pancreas, muscle, heart, kidney, lung, CNS, skin, reproductive tissue, bladder, eye, skeletal, intestinal, or spleen. In some embodiments, the donor cells are derived from embryonic stem cells (ESCs) or induced pluripotent stem cells (iPSCs). In some embodiments, the donor cells are derived from an organ. In some embodiments, the organ is liver, pancreas, kidney, lung, brain, heart, bladder, spleen, intestines, muscle, eye, skin, blood or marrow. In some embodiments, the donor cells are hepatocytes, adipocytes, pancreatic cells, cardiomyocytes, neurons, retinal cells, corneal cells, stromal cells, hematopoietic cells, or skin cells. In some embodiments, the donor cell is an hepatocyte or liver cell. In some embodiments, the donor cell is a B cell. In some embodiments, the donor cells are hepatocyte-like cells, pancreatic progenitor cells, pancreatic beta cells, motor neurons, oligodendrocytes, oligodendrocyte precursor cells, retinal pigment epithelial cells, retinal ganglion cells, retinal progenitor cells, corneal limbal stem cells, corneal endothelial stem cells, stromal stem cells, keratinocytes, cholangiocytes, pericytes, endothelial cells, Kupffer cells, or fibroblasts. In some embodiments, the donor cells are synthetic cells. In some embodiments, the donor cells are modified. In some embodiments, the donor cells are genetically engineered. In some embodiments, the donor cells are genetically engineered to express an isoform of one or more of the specific polypeptides.

In some embodiments according to any of the methods described above, the one or more specific polypeptides is an organ-specific or tissue-specific polypeptide. In some embodiments, the one or more specific polypeptides is a panel of organ-specific or tissue-specific polypeptides. In some embodiments, the one or more specific polypeptides is a ubiquitous polypeptide.

In some embodiments according to any of the methods described above, the recipient is a mammal, such as a human.

In some embodiments according to any of the methods described above, the recipient has a disease or disorder treatable by cell engraftment.

In some embodiments according to any of the methods described above, the specific isoforms are detected by mass spectrometry, ELISA, surface plasmon resonance, affinity-based methods, and/or polypeptide sequencing-based methods. In some embodiments, the one or more polypeptides are enzymatically cleaved prior to analysis to generate a peptide mixture. In some embodiments, the one or more polypeptides are cleaved with one or more enzymes prior to analysis to generate a peptide mixture. In some embodiments, the one or more polypeptides are cleaved with trypsin prior to analysis to generate a peptide mixture. In some embodiments, the one or more polypeptides are cleaved with trypsin and LysC prior to analysis to generate a peptide mixture. In some embodiments, the peptide mixture is separated prior to analysis. In some embodiments, the peptide mixture is separated by high performance liquid chromatography (HPLC) or capillary zone electrophoresis (CZE). In some embodiments, the specific isoforms are detected by mass spectrometry, such as tandem mass spectrometry. In some embodiments, the mass spectrometry includes electrospray ionization (ESI) or matrix-assisted laser desorption ionization (MALDI). In some embodiments, the mass spectrometry is tandem mass spectrometry. In some embodiments, the mass-to-charge ratios of peptide ions will be recorded in an MS1 scan using a mass analyzer capable of high-resolution and accurate mass. In some embodiments, the mass analyzer is an orbitrap or time-of-flight (TOF) mass analyzer. In some embodiments, the sequence of the peptides will be determined by fragmentation spectra. In some embodiments, the peptide ions will be subjected to a fragmentation event in the gas phase. In some embodiments, the peptide ions are subjected to collision-induced dissociation (CID), higher-energy collisional dissociation (HCD), electron transfer dissociation (ETD), ultraviolet photodissociation (UVPD), or a combination thereof.

In some aspects, the invention provides a method for treating an individual in need thereof, the method comprising a) administering donor cells from a donor individual to a recipient individual; b) isolating samples from the recipient individual after administration of the donor cells; and c) detecting specific isoforms of one or more polypeptides in the samples; wherein the donor cells and cells from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor cells in the recipient individual. In some embodiments, the detecting specific isoforms of one or more polypeptides of step c) is by any one of the methods described above.

In some aspects, the invention provides a method for monitoring engraftment of a donor liver cell in a recipient, the method comprising a) isolating samples from the recipient after administration of the donor cell to the recipient; and b) detecting specific isoforms of one or more polypeptides in the samples; wherein the donor liver cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor liver cells in the recipient.

In some embodiments according to any of the methods for monitoring engraftment of donor liver cells in the recipient described above, the one or more polypeptides are enzymatically, chemically, and/or physically cleaved prior to analysis. In some embodiments, the specific isoforms are detected by mass spectrometry. In some embodiments, the one or more polypeptides are enzymatically cleaved prior to analysis to generate a peptide mixture.

In some embodiments, the one or more polypeptides are cleaved with one or more enzymes prior to analysis to generate the peptide mixture. In some embodiments, the one or more polypeptides are cleaved with trypsin prior to analysis to generate the peptide mixture. In some embodiments, the one or more polypeptides are cleaved with trypsin and LysC prior to analysis to generate the peptide mixture.

In some embodiments according to any of the methods for monitoring engraftment of donor liver cells in the recipient described above, the one or more polypeptides is alpha-1-antitrypsin (AAT), vitamin D binding protein (GC), coagulation factor V (F5), transthyretin (TTR), apolipoprotein E (APOE), beta-2-glycoprotein 1 (APOH), or carboxypeptidase N subunit 2 (CPN2). In some embodiments, the one or more polypeptides is a panel of polypeptides. In some embodiments, the panel of polypeptides comprises at least two polypeptides, such as three, four, five, six, seven, eight, nine, ten, or more than ten polypeptides. In some embodiments, the panel of polypeptides comprises two or more of alpha-1-antitrypsin, vitamin D binding protein, coagulation factor V, transthyretin, apolipoprotein E, beta-2-glycoprotein, and carboxypeptidase N subunit 2. In some embodiments, the one or more polypeptides is alpha-1-antitrypsin, wherein the specific isoforms comprise the sequences DTEEEDFHVDQVTTVK (SEQ ID NO:30) and/or DTEEEDFHVDQATTVK (SEQ ID NO:31). In some embodiments, the one or more polypeptides is coagulation factor V, wherein the specific isoforms comprise the sequences LLSLGAGEFK (SEQ ID NO:1) and/or LLSLGAGEFR (SEQ ID NO:2). In some embodiments, the one or more polypeptides is vitamin D binding protein, wherein the specific isoforms comprise the sequences LPDATPTELAK (SEQ ID NO:3), and/or LPEATPTELAK (SEQ ID NO:4), and/or LPAATPTELAK (SEQ ID NO:42). In some embodiments, the one or more polypeptides is transthyretin, wherein the specific isoforms comprise the sequences AADDTWEPFSSGK (SEQ ID NO:5) and/or AADDTWEPFASVK (SEQ ID NO:6). In some embodiments, the one or more polypeptides is apolipoprotein E, wherein the specific isoforms comprise the sequences LGADMEDVCGR (SEQ ID NO:7) and/or LGADMEDVR (SEQ ID NO:8). In some embodiments, the one or more polypeptides is apolipoprotein E, wherein the specific isoforms comprise the sequences LAVYQAGAR (SEQ ID NO:9) and/or CLAVYQAGAR (SEQ ID NO:10). In some embodiments, the one or more polypeptides is beta-2-glycoprotein, wherein the specific isoforms comprise the sequences EHSSLAFWK (SEQ ID NO:11) and/or EHSSLAFSK (SEQ ID NO:12). In some embodiments, the one or more polypeptides is carboxypeptidase N subunit 2, wherein the specific isoforms comprise the sequences LTVSIEAR (SEQ ID NO:13) and/or LTMSIEAR (SEQ ID NO:14).

In some embodiments according to any of the methods for monitoring engraftment of donor liver cells in the recipient described above, the specific isoforms are detected by mass spectrometry, ELISA, surface plasmon resonance, affinity-based methods, and/or polypeptide sequencing-based methods. In some embodiments, the one or more polypeptides are enzymatically cleaved prior to analysis to generate a peptide mixture. In some embodiments, the one or more polypeptides are cleaved with trypsin prior to analysis to generate a peptide mixture. In some embodiments, the peptide mixture is separated prior to analysis. In some embodiments, the peptide mixture is separated by high performance liquid chromatography (HPLC) or capillary zone electrophoresis (CZE). In some embodiments, the specific isoforms are detected by tandem mass spectrometry. In some embodiments, the mass spectrometry includes electrospray ionization (ESI) or matrix-assisted laser desorption ionization (MALDI). In some embodiments, the mass spectrometry is tandem mass spectrometry. In some embodiments, the mass-to-charge ratios of peptide ions will be recorded in an MS1 scan using a mass analyzer capable of high-resolution and accurate mass. In some embodiments, the mass analyzer is an orbitrap or time-of-flight (TOF) mass analyzer. In some embodiments, the sequence of the peptides will be determined by fragmentation spectra in a MS2 scan. In some embodiments, the peptide ions will be subjected to a fragmentation event in the gas phase. In some embodiments, the peptide ions are subjected to collision-induced dissociation (CID), higher-energy collisional dissociation (HCD), electron transfer dissociation (ETD), ultraviolet photodissociation (UVPD), or a combination thereof.

In some aspects, the invention provides a method for treating a liver disorder in an individual in need thereof, comprising administering donor liver cells from a donor individual to the recipient individual in need thereof. In some embodiments, the method further comprises monitoring engraftment of the donor liver cells in the recipient individual, such as according to any of the methods for monitoring engraftment of donor liver cells in the recipient described above. In some embodiments, the method further comprises quantitating the donor liver cells in the recipient individual in need thereof. In some embodiments, quantitating the donor liver cells in the recipient individual in need thereof is performed by obtaining the ratio of donor-specific isoforms to recipient-specific isoforms of the one or more polypeptides. In some embodiments, the ratio of donor-specific isoforms to recipient-specific isoforms of the one or more polypeptides indicates the performance of the donor liver cells in the recipient individual in need thereof. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor liver cells in the recipient individual in need thereof. In some embodiments, the method further comprises obtaining the amount of the donor-specific isoforms of the one or more polypeptides, wherein the amount of the donor-specific isoforms of the one or more polypeptides reflects the quantity of the donor liver cells in the recipient individual in need thereof.

In some aspects, the invention provides a method for treating a liver disorder in an individual in need thereof, the method comprising a) administering donor liver cells (e.g., hepatocytes) from a donor individual to the recipient individual in need thereof; b) isolating samples from the recipient individual after administration of the donor liver cells (e.g., hepatocytes); and c) detecting specific isoforms of one or more polypeptides in the samples; wherein the donor liver cells (e.g., hepatocytes) and liver cells (e.g., hepatocytes) from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor liver cells (e.g., hepatocytes) in the recipient individual in need thereof. In some embodiments, the detecting specific isoforms of one or more polypeptides of step c) is by any one of the methods described herein. In some embodiments, the method further comprises quantitating the donor liver cells in the recipient individual in need thereof. In some embodiments, quantitating the donor liver cells in the recipient individual in need thereof is performed by obtaining the ratio of donor-specific isoforms to recipient-specific isoforms of the one or more polypeptides. In some embodiments, the ratio of donor-specific isoforms to recipient-specific isoforms of the one or more polypeptides indicates the performance of the donor liver cells in the recipient individual in need thereof. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor liver cells in the recipient individual in need thereof. In some embodiments, the method further comprises obtaining the amount of the donor-specific isoforms of the one or more polypeptides, wherein the amount of the donor-specific isoforms of the one or more polypeptides reflects the quantity of the donor liver cells in the recipient individual in need thereof.

In another aspect, the present invention provides a method for monitoring engraftment of a donor B cell in a recipient, the method comprising: a) isolating samples from the recipient after administration of the donor B cell to the recipient; and b) detecting specific isoforms of one or more polypeptides in the samples; wherein the donor B cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of the donor B cell in the recipient.

In some embodiments according to any of the methods for monitoring engraftment of donor B cells in the recipient described above, the specific isoforms are detected by mass spectrometry, ELISA, surface plasmon resonance, affinity-based methods, and/or polypeptide sequencing-based methods. In some embodiments, the one or more polypeptides are enzymatically, chemically, and/or physically cleaved prior to analysis. In some embodiments, the one or more polypeptides are enzymatically cleaved prior to analysis to generate a peptide mixture. In some embodiments, the one or more polypeptides are cleaved with one or more enzymes prior to analysis to generate the peptide mixture. In some embodiments, the one or more polypeptides are cleaved with trypsin prior to analysis to generate the peptide mixture. In some embodiments, the one or more polypeptides are cleaved with trypsin and LysC prior to analysis to generate the peptide mixture. In some embodiments, the peptide mixture is separated prior to analysis. In some embodiments, the peptide mixture is separated by high performance liquid chromatography (HPLC) or capillary zone electrophoresis (CZE).

In some embodiments according to any of the methods for monitoring engraftment of donor B cells in the recipient described above, the specific isoforms are detected by mass spectrometry. In some embodiments, the specific isoforms are detected by tandem mass spectrometry. In some embodiments, the mass spectrometry includes electrospray ionization (ESI) or matrix-assisted laser desorption ionization (MALDI). In some embodiments, the mass spectrometry is tandem mass spectrometry. In some embodiments, the mass-to-charge ratios of peptide ions will be recorded in an MS1 scan using a mass analyzer capable of high-resolution and accurate mass. In some embodiments, the mass analyzer is an orbitrap or time-of-flight (TOF) mass analyzer. In some embodiments, the sequence of the peptides will be determined by fragmentation spectra in a MS2 scan. In some embodiments, the peptide ions will be subjected to a fragmentation event in the gas phase. In some embodiments, the peptide ions are subjected to collision-induced dissociation (CID), higher-energy collisional dissociation (HCD), electron transfer dissociation (ETD), ultraviolet photodissociation (UVPD), or a combination thereof.

In some embodiments according to any of the methods for monitoring engraftment of donor B cells in the recipient described above, the one or more polypeptides are immunoglobulin heavy constant gamma 1 (IGHG1), immunoglobulin heavy constant gamma 2 (IGHG2), immunoglobulin heavy constant alpha 1 (IGHA1), immunoglobulin heavy constant mu (IGHM), and immunoglobulin kappa constant (IGKC). In some embodiments, the one or more polypeptides are a panel of polypeptides. In some embodiments, the panel of polypeptides comprises at least two polypeptides, such as three, four, five, six, seven, eight, nine, ten, or more than ten polypeptides. In some embodiments, the panel of polypeptides comprises two or more of IGHG1, IGHG2, IGHA1, IGHM, and IGKC. In some embodiments, the one or more polypeptides comprise IGHG1, and wherein the specific isoforms comprise the sequence of i) one or more of SEQ ID NOs: 53-56, or ii) SEQ ID NO: 57 and/or SEQ ID NO:58. In some embodiments, the one or more polypeptides comprise IGHG2, and wherein the specific isoforms comprise the sequence of i) SEQ ID NO:59 and/or SEQ ID NO:60, or ii) SEQ ID NO:61 and/or SEQ ID NO:62, or iii) SEQ ID NO:63 and/or SEQ ID NO:64. In some embodiments, the one or more polypeptides comprise IGHA1, and wherein the specific isoforms comprise the sequence of SEQ ID NO:65 and/or SEQ ID NO: 66. In some embodiments, the one or more polypeptides comprise IGHM, and wherein the specific isoforms comprise the sequence of i) SEQ ID NO:67 and/or SEQ ID NO:68, or ii) SEQ ID NO:69 and/or SEQ ID NO:70. In some embodiments, the one or more polypeptides comprise IGKC, and wherein the specific isoforms comprise the sequence of SEQ ID NO:71 and/or SEQ ID NO:72.

In some aspects, the invention provides a method for treating a B cell disorder in an individual in need thereof, comprising administering donor B cells from a donor individual to the recipient individual in need thereof. In some embodiments, the method further comprises monitoring engraftment of the donor B cells in the recipient individual, such as according to any of the methods for monitoring engraftment of donor B cells in the recipient described above. In some embodiments, the method further comprises quantitating the donor B cells in the recipient individual in need thereof. In some embodiments, quantitating the donor B cells in the recipient individual in need thereof is performed by obtaining the ratio of donor-specific isoforms to recipient-specific isoforms of the one or more polypeptides. In some embodiments, the ratio of donor-specific isoforms to recipient-specific isoforms of the one or more polypeptides indicates the performance of the B cells in the recipient individual in need thereof. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor B cells in the recipient individual in need thereof. In some embodiments, the method further comprises obtaining the amount of the donor-specific isoforms of the one or more polypeptides, wherein the amount of the donor-specific isoforms of the one or more polypeptides reflects the quantity of the donor B cells in the recipient individual in need thereof.

In some aspects, the invention provides a method for treating a B cell disorder in an individual in need thereof, the method comprising: a) administering donor B cells from a donor individual to the recipient individual in need thereof; b) isolating samples from the recipient individual after administration of the donor B cells; and c) detecting specific isoforms of one or more polypeptides in the samples; wherein the donor B cells and cells from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of the donor B cells in the recipient individual in need thereof. In some embodiments, detecting specific isoforms of the one or more polypeptides of step c) is according to any of the methods for monitoring engraftment of donor B cells in the recipient described above. In some embodiments, the method further comprises quantitating the donor B cells in the recipient individual in need thereof. In some embodiments, quantitating the donor B cells in the recipient individual in need thereof is performed by obtaining the ratio of donor-specific isoforms to recipient-specific isoforms of the one or more polypeptides. In some embodiments, the ratio of donor-specific isoforms to recipient-specific isoforms of the one or more polypeptides indicates the performance of the B cells in the recipient individual in need thereof. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor B cells in the recipient individual in need thereof. In some embodiments, the method further comprises obtaining the amount of the donor-specific isoforms of the one or more polypeptides, wherein the amount of the donor-specific isoforms of the one or more polypeptides reflects the quantity of the donor B cells in the recipient individual in need thereof.

Also provided are kits for use of any of the methods described above. In some embodiments, the kit further comprises buffers, diluents, filters, needles, and/or syringes for performing any of the methods described above. In some embodiments, the kit further comprises an instruction for performing said method to detect, quantify, and/or monitor cell engraftment in a recipient individual.

### BRIEF DESCRIPTION OF THE DRAWINGS

FIG. 1 is a graph showing the number of peptides having undesired characteristics that were used to exclude variant peptides from the list of candidates.

FIG. 2 is a graph showing the number of peptides that fall into each class of suitable candidates that can be applied for the intended use.

FIG. 3 is a protein abundance plot. Proteins are ranked according to their abundance, determined by mass spectrometry. The six proteins resulting in suitable variant peptides and albumin (ALB) as the highest abundant protein in plasma are highlighted. The concentration of the proteins (right axis) was determined using the known concentration of albumin and the mass spectrometry determined protein intensity.

FIGS. 4A-4D are exemplifying MS2 fragment ion traces of ion traces from ApoH allele peptides. Tryptic peptides of proteins from individual specific alleles were detected by LC-MS. Heavy-isotope labeled peptides of both peptide allele variants were added to tryptic digests of plasma samples from different individuals in triplicates (y-axis). Parallel-reaction-monitoring (PRM) of the heavy peptide standards and their respective light counterpart was applied, to identify and quantify the present peptides of gene alleles in the patient samples. Ion traces for the y8, y7 and y2 or the y8, y7 and y6 fragments are shown

FIG. 5 is a schematic view of genetic allele identification from tryptic peptides of 11 individuals. Tryptic peptides of proteins from individual specific alleles were detected by LC-MS. Heavy-isotope labeled peptides of both peptide allele variants were added to tryptic digests of plasma samples from different individuals in triplicates (y-axis). Parallel-reaction-monitoring (PRM) of the heavy peptide standards and their respective light counterpart was applied, to identify and quantify the present peptides of gene alleles in the patient samples.

F5:
(SEQ ID NO: 2)
LLSLGAGEFR/
(SEQ ID NO: 1)
LLSLGAGEFK.
GC:
(SEQ ID NO: 4)
LPEATPTELAK/
(SEQ ID NO: 3)
LPDATPTELAK.
TTR:
(SEQ ID NO: 6)
AADDTWEPFASVK/
(SEQ ID NO: 5)
AADDTWEPFSSGK.
APOE:
(SEQ ID NO: 8)
LGADMEDVR/
(SEQ ID NO: 9)
LAVYQAGAR.
APOH:
(SEQ ID NO: 12)
EHSSLAFSK/
(SEQ ID NO: 11)
EHSSLAFWK.
CPN2:
(SEQ ID NO: 14)
LTMSIEAR/
(SEQ ID NO: 13)
LTVSIEAR.

FIGS. 6A-6D show APOE variant peptide titration and quantification in a same plasma sample. The plasma sample containing two APOE allele peptides was processed for mass spectrometry (MS). Heavy isotope-labeled variant peptides corresponding to two APOE alleles were spiked into the digested plasma sample. The identity of the allele peptide being analyzed is shown above each figure. FIGS. 6A-6B show constant amounts of heavy isotope-labeled allele peptides (20 fmol) and increasing amounts of plasma peptides were mixed, then the samples were analyzed by MS. FIG. 6A shows analysis of the LGADMEDVR (SEQ ID NO: 8) allele peptide; FIG. 6B shows analysis of the LAVYQAGAR (SEQ ID NO:9) allele peptide. Both allele peptides detected showed similar increases compared to the constant amount of heavy-isotope labeled peptides. While the heavy isotope-labeled peptides were constant in quantitation, the amount of monitored APOE peptides depended on the input amount of plasma peptides. FIGS. 6C-6D show standard curves of heavy isotope-labeled peptide concentrations measured for absolute quantification of plasma peptides. FIG. 6C shows the standard curve for the LGADMEDVR (SEQ ID NO:8) heavy isotope-labeled peptide; FIG. 6D shows the standard curve for the LAVYQAGAR (SEQ ID NO:9) heavy isotope-labeled peptide. FIGS. 6E and 6F show absolute quantification of APOE peptides from peptide intensity using linear regression with the standard curve data from FIGS. 6C-6D, respectively. FIG. 6E shows quantitation of the LGADMEDVR (SEQ ID NO:8) native allele peptide in the plasma sample containing both allele peptides. FIG. 6F shows quantitation of the LAVYQAGAR (SEQ ID NO:9) native allele peptide in the plasma sample containing both allele peptides.

### DETAILED DESCRIPTION OF THE INVENTION

Existing methods for cell tracking that have been used in the clinic require labeling the cells with contrast agents. Such modifications complicate manufacturing, may modify the biology of the cells and are invariably lost after a number of cell divisions. There remains the need of an ideal tracking method that is minimally invasive and involves as little manipulation of the cells as possible. There is also a need in avoiding introduction of foreign reporter genes, the inclusion of which would pose significant regulatory hurdles and possible safety concerns.

To overcome the limitations of existing technologies, the current application, in some aspects, provides an innovative method to directly monitor the function of the transplanted cells by directly quantifying one or more of the polypeptides produced thereby. In some embodiments, the method does not require any manipulation of the cellular material and is non-invasive. In some embodiments, the method is generally applicable in different contexts and to different cell types. In some embodiments, the one or more of the polypeptides described herein or isoforms thereof can be detected at DNA level, RNA level, and/or protein level, or at modifications thereof, such as post-translational modification of an isoform. In some embodiments, any DNA, RNA, and/or protein detection and/or quantification methods can be used herein.

In some aspects, the invention provides methods for detecting a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, the invention provides methods for detecting a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, the invention provides methods for quantitating a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates a quantitative measure of the donor cells compared to the recipient cells or a measure of the performance of the donor cells in the recipient.

In some aspects, the invention provides methods for quantitating a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates a quantitative measure of the donor cells compared to the recipient cells or a measure of the performance of the donor cells in the recipient.

In some aspects, the invention provides methods for monitoring engraftment and/or function of a donor cell in a recipient, the method comprising a) isolating samples from the recipient one or more times after administration of the donor cell to the recipient; and b) detecting specific isoforms of one or more polypeptides in the samples; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of donor cells in the recipient.

In some aspects, the invention provides methods for monitoring engraftment and/or function of a donor cell in a recipient, the method comprising detecting specific isoforms of one or more polypeptides in samples from the recipient; wherein the samples have been obtained from the recipient one or more times after administration of the donor cell to the recipient; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of donor cells in the recipient.

In certain aspects, the methods described herein are useful for assessing and/or monitoring transplantation of autologous donor cells. In some embodiments, wherein the recipient receives autologous donor cells, the method comprises detecting a polypeptide that the autologous donor cells are engineered to express. In such embodiments, the polypeptide of the engineered autologous donor cells is different than polypeptides expressed by the recipient. In some embodiments, the autologous donor cell is modified, such as genetically modified. In some embodiments, the autologous donor cell is genetically engineered to express an isoform of any of the one or more polypeptides described herein. In some embodiments, the autologous donor cell is engineered to express a polypeptide not present in the recipient, such as a functional receptor (e.g., a CAR) or a cytokine. Hence, in some embodiments, autologous donor cells can be identified apart from recipient cells based on the differences of the one or more polypeptides. In some embodiments, the donor cell is modified to contain an aberration in DNA, RNA, and/or protein, such as DNA/RNA mutations (e.g., insertion, deletion, substitution, truncation), post-translation modifications of a protein, epigenetic modification of DNA/protein, etc. Hence, in some embodiments, autologous donor cells can be identified apart from recipient cells based on the detection of the aberration in DNA, RNA, and/or protein. Any known DNA, RNA, and/or protein detection methods can be used, including but not limited to, PCR, RT-PCR, DNA sequencing, RNA sequencing, chromatin immunoprecipitation-sequencing, northern blot, southern blot, western blot, ELISA, mass spectrometry, etc. In some embodiments, the donor cell is engineered to express an exogenous protein or RNA, including but not limited to, reporter (e.g., GFP), engineered protein such as chimeric antigen receptor (CAR), immunocytokine, etc., siRNA, shRNA, crRNA, tracrRNA, guide RNA, Cas protein, etc. Hence in some embodiments, autologous donor cells can be identified apart from recipient cells based on the detection of the exogenous protein or RNA, or the exogenous nucleic acid or vector encoding such exogenous protein or RNA. Any known DNA, RNA, and/or protein detection methods can be used. For example, when the donor cells are autologous, the donor cells can be modified (e.g., genetically modified) in vitro, in vivo, or ex vivo, with any of the above modifications that does not exist in the recipient cells, e.g., expressing a CAR or a different isoform of any of the polypeptides described herein, and the donor cells can be identified apart from recipient cells based on such modification. Such donor cells can be similarly quantified and/or monitored for engraftment and/or function based on such modifications using any of the methods described herein.

In some aspects, the invention provides methods for monitoring engraftment of a donor liver cell in a recipient, the method comprising a) isolating samples from the recipient after administration of the donor liver cell to the recipient; and b) detecting specific isoforms of one or more polypeptides in the samples; wherein the donor liver cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor liver cells in the recipient.

In some aspects, the invention provides methods for treating an individual in need thereof, the method comprising a) administering donor cells from a donor individual to a recipient individual; b) isolating samples from the recipient individual after administration of the donor cells; and c) detecting specific isoforms of one or more polypeptides in the samples; wherein the donor cells and cells from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor cells in the recipient individual.

In some aspects, the invention provides methods for treating a liver disorder in an individual in need thereof, comprising administering donor liver cells from a donor individual to the recipient individual in need thereof. In some embodiments, the method further comprises monitoring engraftment of the donor liver cells in the recipient individual, such as according to any of the methods for monitoring engraftment of donor liver cells in the recipient described herein.

In some aspects, the invention provides methods for treating a liver disorder in an individual in need thereof, the method comprising a) administering donor liver cells (e.g., hepatocytes) from a donor individual to the recipient individual in need thereof; b) isolating samples from the recipient individual after administration of the donor liver cells (e.g., hepatocytes); and c) detecting specific isoforms of one or more polypeptides in the samples; wherein the donor liver cells (e.g., hepatocytes) and liver cells (e.g., hepatocytes) from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor liver cells (e.g., hepatocytes) in the recipient individual in need thereof.

In some aspects, the invention provides methods for monitoring engraftment of a donor B cell in a recipient, the method comprising: a) isolating samples from the recipient after administration of the donor B cell to the recipient; and b) detecting specific isoforms of one or more polypeptides in the samples; wherein the donor B cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of the donor B cell in the recipient.

In some aspects, the invention provides methods for treating a B cell disorder in an individual in need thereof, comprising administering donor B cells from a donor individual to the recipient individual in need thereof. In some embodiments, the method further comprises monitoring engraftment of the donor B cells in the recipient individual, such as according to any of the methods for monitoring engraftment of donor B cells in the recipient described herein.

In some aspects, the invention provides methods for treating a B cell disorder in an individual in need thereof, the method comprising: a) administering donor B cells from a donor individual to the recipient individual in need thereof; b) isolating samples from the recipient individual after administration of the donor B cells; and c) detecting specific isoforms of one or more polypeptides in the samples; wherein the donor B cells and cells from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of the donor B cells in the recipient individual in need thereof.

### Definitions

For purposes of interpreting this specification, the following definitions will apply and whenever appropriate, terms used in the singular will also include the plural and vice versa.

In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

An organ-specific gene/protein as used herein refers to a gene/protein whose expression is significantly elevated in a specific organ. A tissue-specific gene/protein as used herein refers to a gene/protein whose expression is significantly elevated in a specific tissue. An “organ-specific marker” is defined as an organ-specific gene/protein that is also implicated in diseases related to the organ.

The term “prevalent” as used herein refers to an entity that is elevated in levels in an indicated condition/environment, where the elevation in levels can be mild, moderate, significant or highly significant. A polypeptide that is prevalent in a cell type as used herein refers to a polypeptide whose expression level is mildly, moderately, significantly or highly significantly elevated in the cell type compared to a different cell type. A polypeptide that is prevalent in an organ as used herein refers to a polypeptide whose expression level is mildly, moderately, significantly or highly significantly elevated in one or more cell types within the organ as compared to a cell type from another organ.

The term “polynucleotide” or “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and phosphorothioates, and thus can be an oligodeoxynucleoside phosphoramidate (P—NH2) or a mixed phosphoramidate-phosphodiester oligomer. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, oxidation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “isoforms” as used herein, refers to two or more functionally similar polypeptides that have the essentially the same function but not an identical amino acid sequence. In some examples, the isoforms are encoded by different alleles of a single gene. In some examples, the isoforms are encoded by different genes (e.g., by different genes in a gene family).

The term “detecting” is used herein in the broadest sense to include both qualitative and quantitative measurements of a target molecule (e.g., a target polypeptide). Detecting includes identifying the mere presence of the target molecule in a sample as well as determining whether the target molecule is present in the sample at detectable levels.

“Mass spectrometry” refers to the analytical chemistry technique of identifying an amount and/or type of a compound (e.g., a polypeptide) by measuring the mass-to-charge ratio and abundance of ions. The term “mass spectrometry” may be used interchangeably herein.

The term “sample,” as used herein, refers to a composition that is obtained or derived from a subject and/or individual of interest that contains a molecular entity that is to be characterized and/or identified, for example based on physical, biochemical, chemical and/or physiological characteristics. Samples include, but are not limited to, serum, plasma, vitreous fluid, lymph fluid, synovial fluid, follicular fluid, seminal fluid, amniotic fluid, milk, whole blood, blood-derived cells, urine, cerebro-spinal fluid (CSF), saliva, sputum, tears, perspiration, mucus, tumor lysates, tissue extracts such as homogenized tissue, solid tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In some embodiments, a reference sample is obtained from the recipient individual prior to donor cell administration. In yet another embodiment, a reference sample is obtained from a healthy and/or non-diseased part of the body (e.g., tissues or cells) of the recipient individual. In even another embodiment, a reference sample, is obtained from the donor individual.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

The term “substantially the same,” as used herein, denotes a sufficiently high degree of similarity between two numeric values, such that one of skill in the art would consider the difference between the two values to be of little or no biological and/or statistical significance within the context of the biological characteristic measured by said values. The difference between said two values is, for example, less than about 50%, less than about 40%, less than about 30%, less than about 20%, and/or less than about 10% as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by said values. The difference between said two values is, for example, greater than about 10%, greater than about 20%, greater than about 30%, greater than about 40%, and/or greater than about 50% as a function of the value for the reference/comparator molecule.

An “effective amount” of an agent refers to an amount effective, at dosages and for periods of time necessary, to achieve the desired therapeutic result.

A “therapeutically effective amount” refers to an amount of a therapeutic agent to treat or prevent a disease or disorder in a mammal; for example, the amount of donor cells administered to a recipient individual to provide clinical benefit.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed during the course of clinical pathology. Desirable effects of treatment include, but are not limited to, preventing occurrence recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

### Detection of Engraftment and Functionality of Transplanted Cells

### Methods of Detecting Specific Isoforms in a Population of Peptides

This disclosure, in some aspects, provides a method to directly monitor the function of the transplanted cells by directly quantifying one or more of the polypeptides produced thereby. In some embodiments, the one or more polypeptides from the donor is a different isoform of polypeptides produced by cells of the recipient. In some embodiments, the method does not require any manipulation of the cellular material and is non-invasive. In some embodiments, the method is generally applicable in different contexts and to different cell types.

Performing clinical trials is a monumental monetary investment. Whether the investment generates a return or not hinges on the possibility of demonstrating the safety and clinical efficacy of the intervention. However, establishing a mechanistic link between intervention and outcome is exceptionally difficult without a way to monitor the engraftment and functioning of the cells. This is especially true in the case of regenerative medicine, which is currently being deployed to treat severe conditions for which there is no other effective treatment. Specifically, clinical responses from allogeneic transplantation of cells for regeneration (including hepatocyte and pancreatic islet cell transplantation) are transient which can be attributed to, at least partially, the degree and sustainability of cell engraftment. Currently, there is no ideal and accurate method to reliably quantify level of engraftment on an ongoing basis. The current disclosure, in some aspects, provides a robust, inexpensive, and reliable method to track the engraftment and function of transplanted cells.

Tracking polypeptides directly, as opposed to, for instance, mRNA or DNA, can be advantageous because it allows monitoring the functioning of transplanted cells directly whereas DNA or RNA fragments might be detected in blood or other fluids as a result of cell death. Polypeptide measurements in most cases can also be directly related to a variable of clinical interest. Laboratory tests used in clinical practice almost exclusively monitor polypeptides and at present it would be difficult to give a clinical interpretation of an abundance measurement based on DNA or RNA. For instance albumin and insulin can be used to track the function of hepatocytes and pancreatic islet cells respectively. In some embodiments, the cells secrete polypeptides in a bodily fluid that can be accessed by minimally-invasive means including, but not limited to, blood, saliva, cerebrospinal fluid, and urine.

Minimally invasive procedures and approaches can be deployed as long as donor cells to be tracked secrete one or more polypeptide that can be detected in a body fluid. Examples of donor cells suitable to such minimally invasive monitoring include at least pancreatic, liver, cardiac and hematopoietic cells. The methods described herein are suitable for tracking and monitoring transplantation of heterologous cells, which represent a large fraction of regenerative medicine efforts being pursued at this time. With modifications of donor cells described herein, the methods described herein can also be employed to track and monitor transplantation of allogeneic or autologous donor cells.

In some embodiments, the one or more specific polypeptides are organ-specific, tissue-specific and/or cell specific. For example, polypeptides specifically expressed by hepatocytes (e.g., alpha-1-antitrypsin) can be used to monitor engrafted hepatocytes, or polypeptides specifically expressed by pancreatic cells (e.g. insulin) can be used to monitor engrafted pancreatic cells, polypeptides specifically expressed by B cells or plasma cells can be used to monitor engrafted B cells. In some embodiments, the one or more specific polypeptides are part of the extracellular matrix. In some embodiments, the one or more specific polypeptides are secreted polypeptides (e.g., secreted polypeptides destined for plasma). In other embodiments, ubiquitously expressed polypeptides are used to monitor engrafted cells. Examples of ubiquitously expressed polypeptides (e.g., “housekeeping” polypeptides) are polypeptide expressed in most cells including, but not limited to, polypeptides involved in cellular glycolysis and respiration.

In some embodiments, the one or more specific polypeptides of the donor cells are engineered such that they are a different isoform of the same polypeptide of the recipient. For example, gene editing techniques can be used to alter the form of the one or more polypeptides of the donor to be different isomers than the recipient. Gene editing techniques include, but are not limited to, the CRISPR/Cas system, zinc finger nuclease (ZFN) system, or transcription activator-like effector nuclease (TALEN) system. In some embodiments, RNA editing technique can also be used, such as CRISPR/Cas13-based RNA editing (RNA Editing for Programmable A to I Replacement (REPAIR); e.g., see US20210093667), or LEAPER (leveraging endogenous ADAR for programmable editing of RNA; e.g., see US20210310026), the contents of each of which is incorporated herein by reference in their entirety).

The technology can be used for tracking transplanted cells in a regenerative medicine context. Provided, in some aspects, are methods of monitoring transplanted cells directly, and for providing solid causal evidence to any observed clinical outcome. In some aspects, the described capability will be extremely useful in the interpretation of clinical trial data, specifically to evaluation of the sustained engraftment of transplanted cells, with minimally invasive procedures. For mature transplantation technologies already established in clinical practice, the methods described herein may provide additional utility if deployed to monitor the outcome of the cell transplantation procedure.

In some aspects, there is provided a method for detecting a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, there is provided a method for detecting a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, there is provided a method for quantitating a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates the percentage of donor cells in the recipient.

In some aspects, there is provided a method for quantitating a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates the percentage of donor cells in the recipient.

In some aspects, there is provided a method for monitoring engraftment and/or function of a donor cell in a recipient, the method comprising: (a) isolating samples from the recipient one or more times after administration of the donor cell to the recipient; and (b) detecting specific isoforms of one or more polypeptides in the samples; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor cell in the recipient.

In some aspects, there is provide a method for monitoring engraftment and/or function of a donor liver cell (e.g., hepatocyte) in a recipient, the method comprising a) isolating samples from the recipient after administration of the donor liver cell to the recipient; and b) detecting specific isoforms of one or more polypeptides in the samples; wherein the donor liver cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor liver cell in the recipient.

In some aspects, there is provide a method for monitoring engraftment and/or function of a donor B cell in a recipient, the method comprising a) isolating samples from the recipient after administration of the donor B cell to the recipient; and b) detecting specific isoforms of one or more polypeptides in the samples; wherein the donor B cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor B cell in the recipient.

In some aspects, there is provide a method for monitoring engraftment and/or function of a donor cell (e.g., liver cell such as hepatocyte, or B cell) in a recipient, the method comprising detecting specific isoforms of one or more polypeptides in samples from the recipient; wherein the samples have been obtained from the recipient one or more times after administration of the donor cell to the recipient; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor cell in the recipient. In some embodiments, the one or more samples are isolated or obtained from the recipient within at least about one week after administration of the donor cell.

In some aspects, there is provide a method for monitoring engraftment and/or function of a donor cell in a recipient, the method comprising a) isolating samples from the recipient after administration of the donor cell to the recipient; and b) detecting specific isoforms of a panel of polypeptides in the samples; wherein the donor cell and the recipient express different specific isoforms of each of the one or more polypeptides of the panel, and wherein the specific isoforms comprise greater than about 1% (e.g., greater than about any of 1-5%, such as greater than about 5%) of the isoforms of the polypeptides in a population of individuals; wherein detection of donor-specific isoforms of the one or more polypeptides of the panel indicates engraftment and/or function of the donor cell in the recipient. In some embodiments, the panel comprises two, three, four, five, six, seven, eight, nine, ten, or more than ten polypeptides. In some embodiments, the panel of polypeptides is specific for an organ. In some embodiments, the panel is a liver-specific panel comprised of polypeptides that are prevalent in one or more cell types of the liver. In some embodiments, the panel is a pancreas-specific panel comprised of polypeptides that are prevalent in one or more cell types of the pancreas. In some embodiments, the panel is a kidney-specific panel comprised of polypeptides that are prevalent in one or more cell types of the kidney. In some embodiments, the panel is a lung-specific panel comprised of polypeptides that are prevalent in one or more cell types of the lung. In some embodiments, the panel is a central nervous system-specific panel (e.g., a brain-specific panel) comprised of polypeptides that are prevalent in one or more cell types of the central nervous system. In some embodiments, the panel is a heart-specific panel comprised of polypeptides that are prevalent in one or more cell types of the heart. In some embodiments, the panel is a bladder-specific panel comprised of polypeptides that are prevalent in one or more cell types of the bladder. In some embodiments, the panel is a spleen-specific panel comprised of polypeptides that are prevalent in one or more cell types of the spleen. In some embodiments, the panel is an intestine-specific panel comprised of polypeptides that are prevalent in one or more cell types of the intestines. In some embodiments, the panel is a muscle-specific panel comprised of polypeptides that are prevalent in one or more cell types of muscles. In some embodiments, the panel is an eye-specific panel comprised of polypeptides that are prevalent in one or more specific cell types of the eye. In some embodiments, the panel is a skin-specific panel comprised of polypeptides that are prevalent in one or more specific cell types of the skin. In some embodiments, the panel of polypeptides is specific for a cell type. In some embodiments, the panel is a B cell-specific panel comprised of polypeptides that are prevalent in one or more specific cell subtypes of the B cell lineage.

In some embodiments, the donor and the recipient have isoforms of at least one polypeptide in the panel. In some embodiments, the donor and the recipient have isoforms of at least one, two, three, four, five, six, seven, eight, nine, or ten polypeptides in the panel.

In some embodiments, the sample is a plasma sample, a serum sample, a whole blood sample, a lymph sample, a cerebrospinal fluid sample, or a biopsy sample. In some embodiments, the method further comprises the step of removing cells and/or cellular debris from the collected sample. In some embodiments, the sample is a liver sample. In some embodiments, the sample is a blood sample, such as a B cell sample.

In some embodiments, part or all of the samples are frozen. In some embodiments, part of all of the samples are frozen in the presence of stabilizers. In some embodiments, the frozen samples are thawed out for detection for specific isoforms. In some embodiments, the freezing and thawing of sample does not affect detection of the specific isoforms. In some embodiments, the freezing and thawing of sample does not affect the mass spectrometry readings.

### Donor Cells

The disclosure provides, in some aspects, methods to directly monitor the engraftment and function of the transplanted cells by directly quantifying one or more of the polypeptides produced thereby. As used in this specification, a donor cell can refer to a single donor cell or a plurality of donor cells, such as but not limited to a cluster of donor cells or a population of donor cells, or any number of multiple donor cells. The donor cells to be transplanted can include stem cells or terminally differentiated cells. In some embodiments, the donor cells comprises pluripotent, totipotent, or unipotent stem cells. In some embodiments, the donor cells are lineage-committed cells, including lineage-committed stem cells. In some instances, the lineage-committed cells are hemangioblasts or hepatoblasts. In some embodiments, the unmodified donor cells do not have self-renewal potential. In some embodiments, the donor cells are terminally differentiated cells, such as pancreatic beta cells or hepatocytes. In some embodiments, the donor cells are liver cells, such as one or more of hepatocytes (HCs), hepatic stellate cells (HSCs), Kupffer cells (KCs), and liver sinusoidal endothelial cells (LSECs). In some embodiments, the donor cells are blood cells or hematopoietic cells, such as red blood cells (erythrocytes), white blood cells (leukocytes), platelets (thrombocytes), etc. In some embodiments, the donor cells are white blood cells, such as granulocytes (e.g., basophils, eosinophils, neutrophils, mast cells) or agranulocytes (e.g., lymphocytes, monocytes). In some embodiments, the donor cells are lymphocytes, such as B cells, T cell, natural killer (NK) cells, or X lymphocytes. In some embodiments, the donor cells are B cells, such as any B cell subtypes in the B cell lineage, include but are not limited to, plasmablast, plasma cell, lymphoplasmacytoid cell, memory B cell, B-2 cell, B-1 cell, regulatory B (B_{reg}) cell, etc. In some embodiments, the donor cell is a mature B cell, or an activated B cell. In some embodiments, the donor cells are a homogenous cell population (e.g., same cell type or subtype). In some embodiments, the donor cells are a heterogenous cell population, e.g., different cell types or subtypes, or express different engineered components. In some embodiments, the donor cells administered are primary cells directly retrieved from the donor. In some embodiments, the donor cells are retrieved from the donor, and expanded and/or modified before administration into the recipient. In some embodiments, the modifications comprise genetic modifications (e.g., by CRISPR/Cas). In some embodiments, the modifications comprise alteration in cell fate, such as but not limited to dedifferentiation, somatic reprograming, transdifferentiation, spontaneous differentiation, and/or directed differentiation. In some embodiments, the donor cells are expanded and/or modified on an ex vivo matrix before administration into the recipient. In some embodiments, the donor cells have been adapted to tissue culture before administration to recipient.

### Stem Cells

Stem cells have the ability to self-renew and differentiate into various cell types of a tissue. Stem cells are further stratified into different classes mostly based on their potency, i.e. their ability to differentiate. The most “potent” stem cell is the totipotent stem cell, such as spores and zygotes, which can differentiate into all cell types of the organism, including any extraembryonic tissues. Pluripotent stem cells, on the other hand, can generate any fetal or adult cell type but not extraembryonic structures. The two kinds of pluripotent stem cells commonly studied are embryonic stem cells (ESCs) and induced pluripotent stem cells (iPSCs).

Embryonal stem cell (ES) are generally considered to have unlimited self-renewal capabilities and multipotent and/or pluripotent differentiation, thus possessing the capability of developing into any organ, tissue type or cell type of the three germ layers. These cells can be derived from the inner cell mass of the blastocyst, or can be derived from the primordial germ cells from a postimplantation embryo (embryonal germ cells or EG cells). ES and EG cells have been derived from mice, non-human primates as well as humans (Evans et al., 1981; Matsui et al., 1991; Thomson et al., 1995; Thomson et al., 1998; and Shamblott et al., 1998).

In adult bodies, however, the more common stem cells are unipotent or multipotent, meaning their differentiation ability is restricted to one or a few types of progeny cells. Tissue-intrinsic adult human stem cells have been described and characterized in the field. These cells are capable of maintaining, generating, as well as replacing tissue-specific, terminally differentiated cells. For example, adult neural stem cells can differentiate into neurons, glial cells and oligodendrocytes. Stem cells have been identified in most organs and tissues, including “adult stem cells”, i.e., cells (including cells commonly referred to as “progenitor cells”) that can be derived from any source of adult tissue or organ and can replicate as undifferentiated or lineage committed cells and have the potential to differentiate into at least one, preferably multiple, cell lineages. The best characterized are the hematopoietic stem cells. The ultimate hematopoietic stem cell can give rise to any of the different types of terminally differentiated blood cells. This is a mesoderm-derived cell purified based on cell surface markers and functional characteristics (Hill et al., 1996). Also well characterized is the neural stem cell and a number of mesenchymal stem cells derived from multiple sources (Flax et al., 1998; Clarke et al., 2000; Bruder et al., 1997; Yoo et al., 1998; Makino et al., 1999; and Pittenger et al., 1999).

In some embodiments according to any one of the methods described herein, the donor cells are synthetic cells. In some embodiments, the donor cells are primary cells. In some embodiments, the donor cells are subjected to ex vivo culture and expansion before administration into the recipient.

In some embodiments according to any one of the methods described herein, the donor cells are stem cells. In some embodiments, the donor cells are any one of the stem cells described herein. In some embodiments, the donor cells are pluripotent stem cells, including but not limited to ESCs and iPSCs. In some embodiments, the donor cells are ESC- and iPSC-derived cells, including but not limited to ESC- and iPSC-derived stem cells. In some embodiments, the donor cells are multipotent stem cells or unipotent stem cells. In some embodiments, the donor cells are lineage-committed stem cells. In some embodiments, the donor cells are derivatives or progeny cells of any of the stem cells described herein.

In some embodiments, the donor cells are fetal stem cells. In some embodiments, the donor cells are neonatal stem cells. In some embodiments, the donor stem cells are juvenile stem cells. In some embodiments, the stem cells are derived from umbilical cord blood or umbilical tissue. In some embodiments, the stem cells are derived from neonatal foreskin. In some embodiments, the stem cells are derived from dental pulp. In some embodiments, the donor cells are adult stem cells. In some embodiments, the adult stem cells are stromal stem cells. In some embodiments, the adult stem cells are mesenchymal stromal stem cells. In some embodiments, the stem cells are derived from tissues and/or organs. In some embodiments, the stem cells are derived from adipose tissue, bone marrow, umbilical cord tissue, liver, pancreas, muscle, heart, kidney, lung, the central nervous system (CNS), skin, reproductive tissue, bladder, eye, skeletal tissue, intestinal tissue, or spleen. In some embodiments, the stem cells are hematopoietic stem cells (HSCs).

In some embodiments, the donor cells are differentiated from cells of one lineage to cells of another lineage. For example, the donor cells may be hepatocytes or hepatocyte-like cells that were differentiated from adipocyte stem cells. An example of hepatocyte-like cells derived from adipocyte stem cells is described in WO 2015/042125, the content of which is incorporated herein by reference in its entirety.

### Differentiated Cells, Primary Cells

In some embodiments, the donor cells administered are primary cells directly retrieved from the donor. In some embodiments, the donor cells are derived from tissues. In some embodiments, the donor cells are derived from organs. In some embodiments, the donor cells are derived from one or more organs, wherein the organ is liver, pancreas, kidney, lung, brain, heart, bladder, spleen, intestines, muscle, eye, or skin. In some embodiments, the donor cells are hepatocytes, or hepatocyte-like cells. In some embodiments, the donor cells are adipocytes. In some embodiments, the donor cells are cardiomyocytes or cardiomyocyte-like cells. In some embodiments, the donor cells are neurons or neuron-like cells. In some embodiments, the donor cells are motor neurons or motor neuron-like cells. In some embodiments, the donor cells are skeletal muscle cells or skeletal muscle-like cells. In some embodiments, the donor cells are hematopoietic cells, such as T cells, B cells, NK cells, etc. Tissues which include hematopoietic cells are referred herein to as “hematopoietic cell tissues,” include thymus and bone marrow and peripheral lymphoid tissues, such as spleen, lymph nodes, lymphoid tissues associated with mucosa, such as the gut-associated lymphoid tissues, tonsils, Peyer's patches and appendix and lymphoid tissues associated with other mucosa, for example, the bronchial linings. In some embodiments, the donor cells are obtained from hematopoietic cell tissues. In some embodiments, the donor cells are retrieved from the donor, and expanded and/or modified before administration into the recipient.

In some embodiments, the cells retrieved from the donor are completely homogeneous or significantly homogenous. In some embodiments, the cells retrieved from the donor are heterogeneous. In some embodiments, the donor cells are processed to isolate a more homogeneous population. For example, alpha-1-antitrypsin (AAT) or asialoglycoprotein receptor 1 (ASGR1) may be used to isolate hepatocytes from a heterogeneous donor cell population containing hepatocytes. In some embodiments, one or more of CD19, CD20, BCMA (CD269), CD21, CD22, CD23, CD24, CD40, CD72, CD79a, CD79b, and CD138 can be used to isolate B cells from a blood sample.

In some embodiments, the donor cells have homogenous self-renewal potential. In some embodiments, the donor cells have heterogeneous self-renewal potential. In some embodiments, the donor cells have homogenous differentiation capabilities (e.g. ability to differentiate into multiple types of progeny cells). In some embodiments, the donor cells have heterogeneous differentiation capabilities. In some embodiments, the cells from the donor are processed, such as but not limited to purification, to isolate the cells with certain self-renewal potential and/or differentiation potential. In some embodiments, the donor cells are processed to isolate cells with higher self-renewal potential and/or higher differentiation potential. In some embodiments, the donor cells are processed to isolate cells with lower self-renewal potential and/or lower differentiation potential. In some embodiments, the donor cells are processed to isolate cells with intermediate self-renewal potential and/or intermediate differentiation potential. In some embodiments, the process to isolate cells with certain self-renewal potential and/or differentiation potential includes purification by markers, such as but not limited to lineage markers. In some embodiments, the process to isolate donor cells comprises one or more of: antibody affinity purification, fluorescence activated cell sorting (FACS) or magnetic activated cell sorting (MACS). In some embodiments, the process to isolate cells with certain self-renewal potential and/or differentiation potential includes purification by culture conditions. In some embodiments, hepatoblasts and/or hepatic precursors can be purified from hepatic cells obtained from the donor. In some embodiments, adipocyte stem cells can be purified from adipocyte populations obtained from the donor.

In some embodiments, the donor cells are stem cells, wherein the donor cells are expanded and/or directed to differentiate into progeny cells, including intermediate stem cells, precursors and/or terminally differentiated cells before administration to the recipient. As a non-limiting example, neural stem cells from the donor can be directed to differentiate into neuronal stem cells or glial stem cells before administration to the recipient. In some examples, neural stem cells from the donor can be directed to differentiate into terminally differentiated motor neurons before administration to the recipient. In some embodiments, the donor cells can be expanded and/or induced to transdifferentiate into cells of another lineage before administration to the recipient. In some embodiments, donor cells can be reprogrammed into unipotent, multipotent and/or pluripotent stem cells, expanded and directed to redifferentiate into progeny cells of selected lineage before administration to the recipient. In some embodiments, donor cells as used herein include donor cells that directly obtained from donors as well as cells that are expanded, differentiated, transdifferentiated, reprogrammed, reprogrammed-and-redifferentiated, and/or otherwise modified after retrieval from donor but before administration into the recipient. In some embodiments, transdifferentiation, directed differentiation and/or redifferentation comprises using growth factors, signaling pathway effectors and/or culture conditions to mimic in vivo development and maturation. In some embodiments, transdifferentiation, directed differentiation and/or redifferentation comprise contacting the cells with one or more media comprising combinations of growth factors and/or signaling pathway effectors. In some embodiments, donor B cells are differentiated into plasma cells before administration to the recipient. In some embodiments, hepatoblasts are differentiated into hepatocytes before administration to the recipient.

In some embodiments, adipocyte-derived stem cells are obtained from the donor using suction lipectomy (commonly referred to as liposuction). In some embodiments, the adipocyte-derived stem cells are purified. In some embodiments, the adipocyte-derived stem cells are placed into a three dimensional culture (including but not limited to: hanging drop suspension culture, high density culture, spinner flask culture, microcarrier culture, etc.). In some embodiments, the adipocyte-derived stem cells are contacted with a first medium comprising endoderm-inducing factors, including but not limited to WNT3A, Activin A, fibroblast growth factor and/or B27 supplement, to produce precursors to hepatocyte or hepatocyte-like cell. In some embodiments, the adipocyte-derived stem cells are contacted with a first medium comprising midgut and/or foregut endoderm-inducing factors, including but not limited to WNT3A, Activin A, fibroblast growth factor and/or B27 supplement, to produce precursors to hepatocyte or hepatocyte-like cell. In some embodiments, B27 supplement contains one or more of: albumin, catalase, glutathione (reduced), insulin, superoxidase dismutase, holo transferrin, T3 (triiodo-1-thyronine), L-carnitine, ethanolamine, D(+)-galactose, putrescine, sodium selenite, ethanolic stocks, corticosterone, linoleic acid, linolenic acid, lipoic acid, progesterone, retinol acetate, retinol (vitamin A), D, L-alpha tocopherol (vitamin E) and/or D, L-alpha tocopherol acetate. In some embodiments the fibroblast growth factor comprises FGF 1, 2, 3, 4, 5, 6, 7, 8, 9, 10, 16, 17, 18, 19, 20, and/or 22. In some embodiments, the fibroblast growth factor comprises FGF2, FGF4 and/or FGF10. In some embodiments, the precursor to hepatocyte or hepatocyte-like cell is contacted with a second culture medium comprising one or more hepatocyte-inducing factors. In some embodiments, the second culture medium comprising one or more hepatocyte-inducing factors, wherein the one or more hepatocyte-inducing factors comprises one or more of HGF, FGF4, BMP4, DKK-1, WIF-1, Dexamethasone, oncostatin M, and/or DMSO. In some embodiments, the basal culture media is DMEM, RPMI, Iscove's medium, hepatocyte culture medium (HCM) (Lonza, Cat: cc-3198), or any combinations thereof. Methods of deriving hepatocytes or hepatocyte-like cells are described in U.S. Pat. No. 10,004,767B2, the entirety of which are incorporated herein by reference.

In some embodiments, the donor cells are stem cells, wherein after administration the donor cells differentiate into progeny cells, including intermediate stem cells, precursors and/or terminally differentiated cells in the recipient. As a non-limiting example, after administration neural stem cells from the donor can differentiate into neuronal stem cells or glial stem cells in the recipient. In some examples, after administration neural stem cells from the donor can differentiate into terminally differentiated motor neurons. In some embodiments, after administration the donor cells transdifferentiate into cells of another lineage. In some embodiments, after administration the donor cells dedifferentiates. In some embodiments, donor cells as used herein include cells differentiated, transdifferentiated, dedifferentiated or otherwise modified after administration into the recipient.

### Modifications of Donor Cells

In some embodiments according to any one of the methods described herein, the donor cells are synthetic cells. In some embodiments, the donor cells are primary cells. In some embodiments, the donor cells are subjected to ex vivo culture and expansion before administration into the recipient. In some embodiments, the donor cells are genetically engineered. In some examples, donor cells can be genetically engineered to introduce polypeptide variants, such as specific isoforms for detection and monitoring. In some embodiments, the specific isoforms introduced are naturally occurring in a healthy population. In some embodiments, the specific isoforms to be introduced are identified from databases of genomic variations. In some embodiments, the specific isoforms to be introduced are artificially designed. In some embodiments, the specific isoforms introduced do not affect, or do not significantly affect in vivo function and/or ex vivo expansion of the cells to be transplanted. In some examples, donor cells can be genetically engineered to remove and/or replace a defective gene. For example, the mutated sequences in the hemoglobin-beta gene in cells of sickle cell anemia donors can be replaced with wild type sequences. In some embodiments, the mutated sequences in the one or more of Factor VIII, Factor IX, or Factor X gene in cells of hemophilia donors can be replaced with wild type sequences. In some embodiments, the donor cells are genetically engineered with precise gene editing, such as but not limited to methods using gene editing complexes comprising Cas9, TALEN protein, Zinc-finger nuclease (ZFN), mega nuclease, or Cre recombinase. In some embodiments, the gene editing complex comprises a Cas protein or a Cpf1 protein, and comprises a single guide RNA (sgRNA), or a CRISPR RNA (crRNA) and a trans-activating crRNA (tracrRNA). In some embodiments, the gene editing complex comprises a guide RNA (gRNA) and/or donor DNA. The CRISPR-Cas system is known in the art. Non-limiting aspects of this system are described in U.S. Pat. No. 8,697,359, the content of which is incorporated herein by reference in its entirety.

In some embodiments, the donor cells are allogeneic to the recipient. In some embodiments, the donor cells are heterologous to the recipient. In some embodiments, the donor cells are derived from an individual different from the recipient. In some embodiments, the donor cells are autologous to the recipient. In some embodiments, the donor is subsequently the recipient. In some embodiments, adult stem cells or terminally differentiated cells are obtained from the donor, wherein the donor cells are reprogrammed into unipotent, multipotent and/or pluripotent stem cells (such as by introduction of reprogramming factors Oct4, Sox2, Klf4, c-Myc for reprograming to pluripotent stem cells) and directed to redifferentiate into progeny cells of selected lineage before administration to the recipient. In some embodiments, the donor cells are genetically engineered to introduce polypeptide variants, such as one or more specific isoforms for detection and monitoring. In some embodiments, the one or more introduced specific isoforms are not initially present in the donor, but are introduced into the isolated donor cells through said genetic engineering. In some embodiments, the one or more introduced specific isoforms are not present in the recipient. For example, in some embodiments, adult skin cells are isolated from the donor, wherein the skin cells are somatically reprogrammed into iPSCs, which are subsequently expanded and directed to differentiate down the endoderm lineage into target cell types (such as but not limited to hepatic progenitors, pancreatic progenitors and/or pancreatic islet cell precursors). In some embodiments, the iPSCs are genetically engineered to express the one or more specific isoforms not initially present in the donor, before differentiation into target cell types. In some embodiments, these iPSC-derived progeny cells (such as but not limited to hepatic progenitors, pancreatic progenitors and/or pancreatic islet cell precursors) are genetically engineered to express the one or more specific isoforms not initially present in the donor, before administration into the recipient.

### Recipients

Degenerative diseases generally cannot be cured by small or large molecule drugs. However, cell engraftment, including stem cell transplant have been employed with various degrees of success. In addition, ailments causing organ or system failure are sometimes irreversible and require transplant. The liver is an essential organ in the human body, serving roles such as detoxification, plasma protein synthesis, glycogen storage, red blood cell decomposition and bile production. Parallel to the diverse functions and the workload that the liver bears are a variety of debilitating liver diseases. For terminal liver diseases exhibiting liver failure, transplant is the only viable long-term option, but this has been hampered by a chronic shortage of donors and acute transplant rejection. Stem cell-derived hepatocytes (including iPSC-derived hepatocytes) hold much hope for solving these problems.

Type I diabetes (TID) is a complex polygenic autoimmune disease where destruction of insulin-producing beta cells is often near complete upon discovery at a juvenile age. Pancreatic islet cell or stem cell transplantation could replenish beta cells in the patient, and holds promising prospect for TID patients. On the other hand, while Type II diabetes (T2D) cannot be completely cured by stem or islet cell transplant due to existing insulin resistance, patients could still benefit from beta cell replenishment. Nevertheless, clinical responses from allogeneic transplantation of cells for regeneration (including hepatocyte and pancreatic islet cell transplantation) are often transient which is likely due to the degree and sustainability of cell engraftment. The current disclosure, among other things, disclose methods of providing a robust, inexpensive, and reliable method to track the engraftment and function of transplanted cells.

In some embodiments, the recipient is a bird. In some embodiments, the recipient is a mammal. In some embodiments, the recipient is a non-human primate, cat, dog, mouse, rat, rabbit, hamster, or guinea pig. In some embodiments, the recipient is a human. In some embodiments, the recipient has a disease or disorder treatable by cell engraftment. In some embodiments, the recipient has a degenerative disease. In some embodiments, the recipient has a neurodegenerative disease. In some embodiments, neurodegenerative disease has an immune component. In some embodiments, the recipient has a motor neuron disease. In some embodiments, the recipient has a demyelinating disease. In some embodiments, the recipient has Alzheimer's disease, amyotrophic lateral sclerosis (ALS), Huntington's disease, Parkinson's disease, Neuromyelitis optica (NMO), or Multiple Sclerosis. In some embodiments, the recipient has Parkinson's disease. In some embodiments, the recipient has APOE-related disease, e.g., Alzheimer's disease. In some embodiments, the recipient has ocular diseases, such as but not limited to macular degeneration. In some embodiments, the recipient has a genetic disease. In some embodiments, the recipient has a disease with genetic predisposition. In some embodiments, the recipient is genetically disposed to develop the disease. In some embodiments, the recipient has sickle cell anemia. In some embodiments, the recipient has hemophilia. In some embodiments, the recipient has thalassemia (e.g., β thalassemia), Fanconi anemia, and/or aplastic anemia. In some embodiments, the recipient has any one of cystic fibrosis, progressive pseudohypertrophic muscular dystrophy, Becker muscular dystrophy (BMD), alpha-1-antitrypsin deficiency (A1AD, or AAT deficiency), transthyretin-related amyloidosis (ATTR), Pompe disease, myotonic dystrophy, fragile X syndrome (FXS), Friedreich ataxia (FRDA), hereditary angioedema (HAE), frontotemporal dementia (FTD), hereditary chronic kidney disease, hyperlipidemia, hypercholesterolemia, Leber congenital amaurosis (LCA). In some embodiments, the disease or condition is ATTR, such as transthyretin-related wild-type amyloidosis (ATTRwt), transthyretin-related hereditary amyloidosis (ATTRm), familial amyloid polyneuropathy (FAP, ATTR-PN), or familial amyloid cardiomyopathy (FAC, ATTR-CM). In some embodiments, the disease or condition is transthyretin instability caused by abnormal expression (e.g., high expression) of the TTR gene. In some embodiments, the disease or condition is other disease or condition or derived disease or condition caused by abnormal expression (e.g., high expression) of the TTR gene. In some embodiments, the recipient has one or more immunodeficiency diseases, including but not limited to, AIDS. In some embodiments, the recipient has chronic granulomatous disease (GCD). In some embodiments, the recipient has severe combined immunodeficiency (SCID). In some embodiments, the recipient has impaired cardiac tissue, such as but not limited to cardiac injury. In some embodiments, the recipient has a metabolic disease. In some embodiments, the recipient has one or more liver diseases. In some embodiments, the recipient has acute liver failure or chronic liver failure. In some embodiments, the recipient has an autoimmune disease. In some embodiments, the recipient has a pancreatic disease. In some embodiments, the recipient has TID and/or T2D. In some embodiments, the recipient has cancer or cancer-associated disease. In some embodiments, the cancer is acute lymphoblastic leukemia (ALL), acute myeloid leukemia (AML), chronic lymphocytic leukemia (CLL), chronic myeloid leukemia (CML), Hodgkin's lymphoma, non-Hodgkin's lymphoma, head and neck cancer, cervical cancer, vaginal cancer, ovarian cancer, oral cancer, salivary cancer, breast cancer, lung epithelial cancer, peritoneal cancer, colon cancer, pancreatic cancer, gastric carcinoma, gastrointestinal cancer, liver cancer, hepatocarcinoma, renal cell carcinoma, neuroblastoma or glioblastoma. In some embodiments, the recipient has B cell lymphoma, including but is not limited to, diffuse large B cell lymphoma (DLBCL), Burkitt lymphoma, chronic lymphocytic leukemia (CLL), myeloid leukemia (e.g., AML or CML), lymphoid leukemia (e.g., ALL, myelodysplasia), small lymphocytic lymphoma (SLL), follicular lymphoma (FL), mantle cell lymphoma (MCL), marginal zone lymphoma, lymphoplasmacytic lymphoma (Waldenstrom macroglobulinemia), hairy cell leukemia (HCL), primary CNS lymphoma, primary intraocular lymphoma, mucosa-associated-lymphoid tissue B cell lymphoma. In some embodiments, the B cell lymphoma is non-Hodgkin's lymphoma (NHL). In some embodiments, the B cell lymphoma is Hodgkin's lymphoma. In some embodiments, the B cell lymphoma is indolent (slow-growing). In some embodiments, the B cell lymphoma is aggressive (fast-growing). In some embodiments, B cell lymphoma is relapsed aggressive, relapsed indolent, refractory, or refractory indolent.

Polypeptides with Isoforms

In some embodiments, the one or more polypeptides are ubiquitous. In some embodiments, the one or more polypeptides are “house-keeping” polypeptides, such as actin. In some embodiments, the one or more polypeptides are germ layer-specific. In some embodiments, the one or more polypeptides are prevalent in a germ layer. In some embodiments, the one or more polypeptides are cell lineage-specific. In some embodiments, the one or more polypeptides are prevalent in a cell lineage, e.g., B-cell lineage. In some embodiments, the one or more polypeptides are tissue-specific. In some embodiments, the one or more polypeptides are prevalent in a tissue. In some embodiments, the one or more polypeptides are organ-specific, e.g., liver-specific. In some embodiments, the one or more polypeptides are prevalent in an organ. In some embodiments, the one or more polypeptides are specific to liver. In some embodiments, the one or more polypeptides are prevalent in the liver. In some embodiments, the one or more polypeptides are specific to hepatoblasts, hepatocytes and/or hepatocyte-like cells. In some embodiments, the one or more polypeptides are prevalent in the hepatoblasts, hepatocytes and/or hepatocyte-like cells. In some embodiments, the one or more polypeptides are secreted by hepatoblasts, hepatocytes or hepatocyte-like cells. In some embodiments, the one or more polypeptides comprises alpha-1-antitrypsin (also known as alpha1-proteinase inhibitor, alpha1-antiproteinase, AAT), vitamin D binding protein (DBP, GC, VDB, GRD3, VDBG, GcMAF, DBP/GC, DBP-maf, HEL-S-51), coagulation factor V (F5, Factor V), transthyretin (TTR, TBPA), apolipoprotein E (APOE), beta-2-glycoprotein 1 (apolipoprotein H, APOH, beta-2 GP1), carboxypeptidase N subunit 2 (CPN2), serum albumin, apolipoprotein A-I (Apo-AI, APOA1), apolipoprotein A-II (APOA2, Apo-AII), haptoglobin, complement C3, fibrinogen beta chain, fibrinogen alpha chain, fibrinogen gamma chain, and/or vitamin B binding protein. In some embodiments, the methods of the invention use a panel of two or more polypeptides selected from alpha-1-antitrypsin, vitamin D binding protein, coagulation factor V, transthyretin, apolipoprotein E, beta-2-glycoprotein, carboxypeptidase N subunit 2, serum albumin, apolipoprotein A-I, apolipoprotein A-II, haptoglobin, complement C3, fibrinogen beta chain, fibrinogen alpha chain, fibrinogen gamma chain, and vitamin B binding protein. In some embodiments, the methods of the invention use a panel of three or more polypeptides, four or more polypeptides, five or more polypeptides, six or more polypeptides, seven or more polypeptides, eight or more polypeptides, nine or more polypeptides, ten or more polypeptides, eleven or more polypeptides, twelve or more polypeptides, thirteen or more polypeptides, or fourteen or more polypeptides selected from alpha-1-antitrypsin, vitamin D binding protein, coagulation factor V, transthyretin, apolipoprotein E, beta-2-glycoprotein, carboxypeptidase N subunit 2, serum albumin, apolipoprotein A-I, apolipoprotein A-II, haptoglobin, complement C3, fibrinogen beta chain, fibrinogen alpha chain, fibrinogen gamma chain, and/or vitamin B binding protein. In some embodiments, the methods of the invention use a panel of polypeptides comprising alpha-1-antitrypsin, vitamin D binding protein, coagulation factor V, transthyretin, apolipoprotein E, beta-2-glycoprotein, carboxypeptidase N subunit 2, serum albumin, apolipoprotein A-I, apolipoprotein A-II, haptoglobin, complement C3, fibrinogen beta chain, fibrinogen alpha chain, fibrinogen gamma chain, and vitamin B binding protein. In some embodiments, the one or more polypeptides are produced by B cells (e.g., any subtypes in the lineage), such as plasma cells. In some embodiments, the one or more polypeptides comprises immunoglobulin heavy constant gamma 1 (IGHG1), immunoglobulin heavy constant gamma 2 (IGHG2), immunoglobulin heavy constant alpha 1 (IGHA1), immunoglobulin heavy constant mu (IGHM), immunoglobulin kappa constant (IGKC), and immunoglobulin lambda constant (IGLC2). In some embodiments, the methods of the invention use a panel of two or more polypeptides selected from IGHG1, IGHG2, IGHA1, IGHM, IGKC, and IGLC2. In some embodiments, the methods of the invention use a panel of three or more polypeptides, four or more polypeptides, five or more polypeptides, such as six polypeptides, selected from IGHG1, IGHG2, IGHA1, IGHM, IGKC, and IGLC2. In some embodiments, the panel of polypeptides comprises two or more of IGHG1, IGHG2, IGHA1, IGHM, and IGKC, such as all of IGHG1, IGHG2, IGHA1, IGHM, and IGKC.

In some embodiments, the one or more polypeptides are specific to pancreas. In some embodiments, the one or more polypeptides are prevalent in pancreatic progenitors and/or pancreatic beta cells. In some embodiments, the one or more polypeptides are secreted by pancreatic beta cells. In some embodiments, the one or more polypeptides comprises insulin.

In some embodiments, the one or more polypeptides are prevalent in motor neurons. In some embodiments, the one or more polypeptides are prevalent in oligodendrocytes and/or oligodendrocyte precursors. In some embodiments, the one or more polypeptides are prevalent in retinal pigment epithelial cells. In some embodiments, the one or more polypeptides comprises bestrophin. In some embodiments, the one or more polypeptides are prevalent in retinal progenitors. In some embodiments, the one or more polypeptides are prevalent in retinal ganglion cells. In some embodiments, the one or more polypeptides are prevalent in stromal stem cells. In some embodiments, the one or more polypeptides are prevalent in corneal limbal stem cells. In some embodiments, the one or more polypeptides are prevalent in corneal endothelial stem cells. In some embodiments, the one or more polypeptides are prevalent in keratinocytes. In some embodiments, the one or more polypeptides are prevalent in cardiomyocytes. In some embodiments, the one or more polypeptides are prevalent in hematopoietic cells. In some embodiments, the one or more polypeptides are prevalent in neurons. In some embodiments, the one or more polypeptides are prevalent in adipocytes. In some embodiments, the one or more polypeptides are prevalent in adipocytes are prevalent in cholangiocytes, pericytes, endothelial cells, Kupffer cells, and/or fibroblasts.

In some embodiments, the invention provides a panel of polypeptides for detecting donor cells for use in the methods of the invention. In some embodiments, the panel comprises two, three, four, five, six, seven, eight, nine, ten, or more than ten polypeptides. In some embodiments, the panel of polypeptides is specific for an organ. In some embodiments, the panel is a liver-specific panel comprised of polypeptides that are prevalent in one or more cell types of the liver. In some embodiments, the panel is a pancreas-specific panel comprised of polypeptides that are prevalent in one or more cell types of the pancreas. In some embodiments, the panel is a kidney-specific panel comprised of polypeptides that are prevalent in one or more cell types of the kidney. In some embodiments, the panel is a lung-specific panel comprised of polypeptides that are prevalent in one or more cell types of the lung. In some embodiments, the panel is a central nervous system-specific panel (e.g., a brain-specific panel) comprised of polypeptides that are prevalent in one or more cell types of the central nervous system. In some embodiments, the panel is a heart-specific panel comprised of polypeptides that are prevalent in one or more cell types of the heart. In some embodiments, the panel is a bladder-specific panel comprised of polypeptides that are prevalent in one or more cell types of the bladder. In some embodiments, the panel is a spleen-specific panel comprised of polypeptides that are prevalent in one or more cell types of the spleen. In some embodiments, the panel is an intestine-specific panel comprised of polypeptides that are prevalent in one or more cell types of the intestines. In some embodiments, the panel is a muscle-specific panel comprised of polypeptides that are prevalent in one or more cell types of muscles. In some embodiments, the panel is an eye-specific panel comprised of polypeptides that are prevalent in one or more specific cell types of the eye. In some embodiments, the panel is a skin-specific panel comprised of polypeptides that are prevalent in one or more specific cell types of the skin. In some embodiments, the panel is a B cell-specific panel comprised of polypeptides that are prevalent in one or more subtypes of the B cell lineage, e.g., plasma cells.

In some embodiments, expression of the one or more polypeptides by the administered donor cells indicates engraftment of the donor cells. In some embodiments, expression of the one or more polypeptides by the administered donor cells indicates function of the donor cells. In some embodiments, detection of the one or more polypeptides by the administered donor cells indicates engraftment and/or function of the donor cells. In some embodiments, ratio of donor-specific isoforms to recipient-specific isoforms of the one or more polypeptides indicates the percentage of donor cells in the recipient. In some embodiments, degree of expression of the one or more polypeptides by the administered donor cells is used as an indicator on engraftment of the donor cells. In some embodiments, degree of expression of the one or more polypeptides by the administered donor cells is used as an indicator of function of the donor cells. In some embodiments, the ratio of donor-specific isoforms to recipient-specific isoforms is about any one of 1,000,000:1, 100,000:1, 10,000:1, 5000:1, 1000:1, 500:1, 300:1, 200:1, 100:1, 80:1, 50:1, 30:1, 20:1, 10:1, 5:1, 2:1, 1:1, 1:2, 1:5, 1:10, 1:20, 1:30, 1:50, 1:80, 1:100, 1:200, 1:300, 1:500, 1:1000, 1:10,000, 1:100,000 or 1:1,000,000. In some embodiments, the amount of donor-specific isoform(s) of the one or more polypeptides is any one of about: 0.0001%, 0.0005%, 0.001%, 0.005%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 50%, 60%, 70%, 80%, 90%, 95%, 99%, or 100% of the recipient-specific isoform(s) in the collected sample(s). In some embodiments, the amount of donor-specific isoform(s) of the one or more polypeptides is any one of about: 2-fold, 3-fold, 4-fold, 5-fold, 8-fold, 10-fold, 15-fold, 20-fold, 30-fold, 40-fold, 50-fold, 60-fold, 70-fold, 80-fold, 90-fold, 100-fold or more than the recipient-specific isoform(s) in the collected sample(s). In some embodiments, the amount of donor-specific isoform(s) of the one or more polypeptides is at or greater than the detection limit by mass spectrometry.

In some embodiments, the specific isoforms comprise greater than about 1% (such as greater than about any of 2%, 3%, 4%, 5%, or more) of the isoforms of the polypeptide in a population of individuals. In some embodiments, the specific isoforms comprise greater than 1-5% of the isoforms of the polypeptide in a population of individuals. In some embodiments, the specific isoforms comprise greater than about 5% of the isoforms of the polypeptide in a population of individuals. In some embodiments, the specific isoforms comprise greater than about any one of 0.001%, 0.005%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 95%, or more of the isoforms of the polypeptide in a population of individuals. In some embodiments, the specific isoforms comprise any one of about 0.001% to about 95%, about 0.001% to about 80% of the isoforms of the polypeptide in a population of individuals. In some embodiments, the specific isoforms comprise any one of about 0.001% to about 0.005%, about 0.005% to about 0.01%, about 0.01% to about 0.05%, about 0.05% to about 0.1%, about 0.1% to about 0.5%, about 0.5% to about 1%, about 1% to about 5%, about 5% to about 8%, about 8% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 60%, about 60% to about 70%, about 70% to about 80%, about 80% to about 90%, or about 90% to about 95%, of the isoforms of the polypeptide in a population of individuals. In some embodiments, the population of individuals refers to a general population. In some embodiments, the population of individuals refers to a non-diseased general population. In some embodiments, the population of individuals refers to a diseased general population. In some embodiments, the population of individuals refers to general population of a specific gender. In some embodiments, the population of individuals refers to general population of a specific race. In some embodiments, the population of individuals refers to general population of a specific geographical region. In some embodiments, the population of individuals refers to general population of a specific age range.

In some embodiments, the specific isoforms expressed by the donor cells comprise greater than about 1% (such as greater than about any of 2%, 3%, 4%, 5%, or more) of the isoforms of the polypeptide in a population of individuals. In some embodiments, the specific isoforms expressed by the donor cells comprise greater than 1-5% of the isoforms of the polypeptide in a population of individuals. In some embodiments, the specific isoforms expressed by the donor cells comprise greater about 5% of the isoforms of the polypeptide in a population of individuals. In some embodiments, the specific isoforms expressed by the donor cells comprise greater than about any one of 0.001%, 0.005%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 95%, or more of the isoforms of the polypeptide in a population of individuals. In some embodiments, the specific isoforms expressed by the donor cells comprise any one of about 0.001% to about 0.005%, about 0.005% to about 0.01%, about 0.01% to about 0.05%, about 0.05% to about 0.1%, about 0.1% to about 0.5%, about 0.5% to about 1%, about 1% to about 5%, about 5% to about 8%, about 8% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 60%, about 60% to about 70%, about 70% to about 80%, about 80% to about 90%, or about 90% to about 95%, of the isoforms of the polypeptide in a population of individuals. In some embodiments, the population of individuals refers to a general population. In some embodiments, the population of individuals refers to a non-diseased general population. In some embodiments, the population of individuals refers to a diseased general population. In some embodiments, the population of individuals refers to general population of a specific gender. In some embodiments, the population of individuals refers to general population of a specific race. In some embodiments, the population of individuals refers to general population of a specific geographical region. In some embodiments, the population of individuals refers to general population of a specific age range.

In some embodiments, the specific isoforms expressed by the donor cells and the recipient cells comprise greater than about 1% (such as greater than about any of 2%, 3%, 4%, 5%, or more) of the isoforms of the polypeptide in a population of individuals. In some embodiments, the specific isoforms expressed by the donor cells and the recipient cells comprise greater than 1-5% of the isoforms of the polypeptide in a population of individuals. In some embodiments, the specific isoforms expressed by the donor cells and the recipient cells comprise greater than about 5% of the isoforms of the polypeptide in a population of individuals. In some embodiments, the specific isoforms expressed by the donor cells and the recipient comprise greater than about any one of 0.001%, 0.005%, 0.01%, 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 95%, or more of the isoforms of the polypeptide in a population of individuals. In some embodiments, the specific isoforms expressed by the donor cells and the recipient comprise any one of about 0.001% to about 0.005%, about 0.005% to about 0.01%, about 0.01% to about 0.05%, about 0.05% to about 0.1%, about 0.1% to about 0.5%, about 0.5% to about 1%, about 1% to about 5%, about 5% to about 8%, about 8% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 60%, about 60% to about 70%, or about 70% to about 80%, about 80% to about 90%, or about 90% to about 95%, of the isoforms of the polypeptide in a population of individuals. In some embodiments, the population of individuals refers to a diseased general population. In some embodiments, the population of individuals refers to general population of a specific gender. In some embodiments, the population of individuals refers to general population of a specific race. In some embodiments, the population of individuals refers to general population of a specific geographical region. In some embodiments, the population of individuals refers to general population of a specific age range.

In some embodiments, the samples are isolated or obtained within about one week, about two weeks, about three weeks, about one month, about two months, about three months, about four months, about five months, about six months, about nine months, about one year, or more than one year after engraftment or administration of the donor cells.

In some embodiments, the samples were obtained for one or more times after administration of the donor cells. In some embodiments, the samples were obtained for about any one of 1, 2, 3, 4, 5, 6, 7, 8, 9, 10, 11, 12, 13, 14, 15, 16, 17, 18, 19, 20, 25, 50, 75, 100 or more times after administration of the donor cells. In some embodiments, the first sample is isolated or obtained within about one week, about two weeks, about three weeks, about one month, about two months, about three months, about four months, about five months, about six months, about nine months, about one year, or more than one year after engraftment or administration of the cells. In some embodiments, the successive samples are isolated or obtained about within about one week, about two weeks, about three weeks, about one month, about two months, about three months, about four months, about five months, about six months, about nine months, about one year, or more than one year after isolation or obtainment of the prior sample.

### Samples Containing Polypeptides

In some aspects, the method described herein comprises obtaining polypeptide-containing sample such as a fluid, cell, tissue or organ sample. In some embodiments, the polypeptide-containing sample is from bone marrow, blood, liver, or spleen. A polypeptide-containing sample of the present invention may be obtained from the donor and/or the transplant recipient by various means.

In some embodiments, the polypeptide-containing sample is a bodily fluid that can be accessed by minimally-invasive means including, but not limited to, blood (including whole blood, plasma and serum), lymph, saliva, cerebrospinal fluid, urine, interstitial fluids, vitreous fluid, synovial fluid, follicular fluid, seminal fluid, amniotic fluid, milk, saliva, sputum, tears, perspiration, mucus, synovial fluid, vitreous humor, or aqueous humor. In some embodiments, blood or serum can be obtained. One method of collecting a blood or serum sample may comprise venipuncture, where blood is drawn directly from a blood vessel in the arm of an individual through a needle placed in a single vein. The blood may then be collected in a glass, or plastic tube or other containers. In some embodiments, saliva can be collected by swabbing. In some embodiments, urine can be obtained from urine samples.

Cerebrospinal fluid (CSF) is a clear, colorless body fluid found in the brain and spinal cord. It is produced by specialized ependymal cells in the choroid plexuses of the ventricles of the brain, and absorbed in the arachnoid granulations. There is about 125 mL of CSF at any one time, and about 500 mL is generated every day. CSF acts as a cushion or buffer, providing basic mechanical and immunological protection to the brain inside the skull. In some embodiments, CSF is obtained via lumbar puncture (also known as spinal tap). In some embodiments, lumbar puncture procedure involves local anesthesia and sterile technique. In some embodiments, a hypodermic needle is used to access the subarachnoid space for collection of CSF.

Aspiration biopsy, also referred to as Fine Needle Aspiration (FNA), is performed with a fine needle attached to a syringe. Aspiration biopsy or FNA may be employed to obtain a polypeptide-containing sample. In some instances, FNA biopsy is a percutaneous (through the skin) biopsy. FNA biopsy is typically accomplished with a fine gauge needle. The area is first cleansed and then usually numbed with a local anesthetic. The needle is placed into the region of organ, tissue or interstitial space of interest. Once the needle is placed a vacuum is created with the syringe and multiple in and out needle motions are performed. In some embodiments, the vacuum is created by aspiration or vacuum pump. The cells or fluids to be sampled are then sucked into the syringe through the fine needle. In some instances, three or four or more samples are obtained.

Organs or interstitial spaces that are not easily reached such as the pancreas, lung, and liver or surrounding interstitial spaces are good candidates for FNA. FNA procedures are typically done using ultrasound or computed tomography (CT) imaging.

In some embodiments, cell, organ or tissue sample of the invention may be obtained by a biopsy. A biopsy is the removal of a sample from the body. Biopsies that may be employed in the present invention include punch biopsy, cone biopsy, needle biopsy, endoscopic biopsy or surface biopsy but are not limited to such.

Punch biopsy is typically used to obtain samples of skin rashes, moles, small tissue samples from skin, cervix, or from other small masses. After a local anesthetic is injected, a biopsy punch, (in some instances 3 mm to 4 mm or 0.15 inch in diameter), is used to cut out a cylindrical piece of skin. The opening is typically closed with a suture and heals with minimal scarring. Core needle biopsy (or core biopsy) is performed by inserting a small hollow needle through the skin and into the organ. The needle is then advanced within the cell layers to remove a sample or core. The needle may be designed with a cutting tip to help remove the sample of tissue. Core biopsy is often performed with the use of a spring loaded gun to help remove the tissue sample. Core biopsy can be performed under image guidance such as CT imaging, ultrasound or mammography. The needle is either placed by hand or with the assistance of a sampling device. Multiple insertions can be made to obtain sufficient tissue, and multiple samples can be taken. Endoscopic biopsy is a common type of biopsy that may be employed in to obtain a polypeptide-containing sample. Endoscopic biopsy is done through an endoscope (a fiber optic cable for viewing inside the body) which is inserted into the body along with sampling instruments. The endoscope allows for direct visualization of an area on the lining of the organ of interest. Samples are obtained by collection or pinching off of tiny bits of tissue with forceps attached to a long cable that runs inside the endoscope of the sample. Endoscopic biopsy may be performed on the gastrointestinal tract (alimentary tract endoscopy), urinary bladder (cystoscopy), abdominal cavity (laparoscopy), joint cavity (arthroscopy), mid-portion of the chest (mediastinoscopy), or trachea and bronchial system (laryngoscopy and bronchoscopy). In some embodiments, endoscopic biopsy can be performed either through a natural body orifice or a small surgical incision. Surface biopsy may be employed to obtain a polypeptide-containing sample. This technique involves sampling or scraping of the surface of a tissue or organ to remove cells. Surface biopsy is often performed to remove a small piece of skin.

### Selection of Specific Polypeptides

In some aspects, the invention provides means for selecting specific polypeptides for use in the methods of the invention. In some embodiments, the methods include selecting abundant proteins in the cell, tissue, or organ sample, such as abundant plasma proteins. Lists of proteins found in plasma are available in the public domain (e.g., Anderson Mol Cell Proteomics 2002). In some embodiments, the proteins in these lists are categorized by the cell type they are predominantly synthesized by, such as B lymphocytes (e.g., plasma cells) and hepatocytes. In some embodiments, the list or proteins (e.g., plasma proteins) are ordered by their respective concentrations in the cell, tissue, or organ obtained, such as concentrations in plasma.

In some embodiments, the abundancy of plasma proteins is verified by RNA abundance. For example, the plasma protein abundance, as ordered above, is subsequently validated using the Human Protein Atlas (https://www.proteinatlas.org/) which contains abundance of RNA in different tissues. In some embodiments, the database is searched for cell-specific (e.g., B-cell specific) or organ-specific (e.g., liver specific) proteins, and the corresponding RNA abundance from the Human Protein Atlas is used to verify the relative abundance of the proteins as identified using the protein database(s). In some embodiments, the protein (e.g., based on above protein abundance) is verified and/or sorted by corresponding Single Cell Type RNA abundance.

In some embodiments, the methods of the invention include the identification of prevalent amino acid variants; for example, proteins (as verified in a combined list based on protein abundance and RNA abundance above) that are ranked in the top half in the list in terms of abundances (above about the 50th percentile) are further analyzed for naturally occurring amino acid variants using the UniProt database (https://www.uniprot.org/). In some embodiments, proteins that are ranked above about the 55^{th}, 60^{th}, 65^{th}, 70^{th}, 75^{th}, 80^{th}, 90^{th} or 95^{th} percentile are further analyzed. In some embodiments, all proteins identified (e.g., based on protein abundance and/or RNA abundance above) are further analyzed. In some embodiments, for each protein the raw data on the naturally occurring amino acid variants is downloaded and filtered for variants with a population prevalence of greater than about any of 1%, 5%, 10%, 15%, 20%, 25%, 50%, or higher. In some embodiments, the corresponding allelic frequencies for each protein variant are also determined from one or more of the following databases: genomeADbrowser (world wide web gnomad.broadinstitute.org/), NHLBI Trans-Omics for Precision Medicine (TOPMED, world wide web nhlbiwgs.org/), and ExAC Browser (world wide web re3data.org/repository/r3d100012122).

In some embodiments, proteins (e.g., based on protein abundance and/or RNA abundance above) are mapped to corresponding genes using genomeADbrowser. In some embodiments, missense mutations in the mapped genes are identified (e.g., using genomeADbrowser), then ordered from high to low by frequency of missense mutations. In some embodiments, genes with a population mutation frequency of greater than about any of 1%, 5%, 10%, 15%, 20%, 25%, 50%, or higher (such as greater than 5%) are selected for further analysis. In some embodiments, genes with a population mutation frequency of greater about any of 1%, 5%, 10%, 15%, 20%, 25%, 50%, or higher (such as greater than 5%) identified are further analyzed for variant population frequency, such as by searching in the NCBI dbSNP database (https://www.ncbi.nlm.nih.gov/snp/). In some embodiments, the associated polypeptides of the alleles are further identified. In some embodiments, sequences of amino acid variants with population frequency (or population prevalence) above 5% are obtained, such as from the UniProt database (https://www.uniprot.org/), genomeADbrowser, or any other protein sequence database.

In some embodiments, the methods of the invention include an iterative selection of prevalent variants within abundant proteins, such as abundant plasma proteins. This step can facilitate continued searching for candidate proteins in a long list of candidates. For example, if the first X candidate proteins are analyzed and do not yield the desired variants, one can iteratively go back to the initial list of candidates based on protein abundance to find additional candidate proteins to analyze. In some embodiments, after determining the population frequency of the amino acid variants in the list of proteins from above, more than about 5, 10, 15, 20, 25, or 50 variants may be selected as follows. Starting with the proteins of highest abundance in the cell, tissue, or organ sample, such as plasma, the known variants are examined. If any of the proteins had amino acid variants with population frequency above about 1%, 5%, 10%, 15%, 20%, 25%, or 50%, that corresponding variant is selected. If a protein had no amino acid variants with population frequency greater than about 1% (e.g., greater than about any of 1-5%, such as greater than about 5%), the search moves to the protein with the next highest abundance in the cell, tissue, or organ sample, such as plasma. In some embodiments, the process is repeated until more than about 5, 10, 15, 20, 25. 30, 35, 40, 45, or 50 amino acid variants are selected. The process could be continued to generate additional variants with frequencies greater than about 1% (e.g., greater than about any of 1-5%, such as greater than about 5%). The population frequency threshold at 1-5% could be modified according to needs and specific applications. For example, population frequency threshold could be preset at 1%, 2%, 3%, 4%, 5%, 6%, 7%, 8%, 9%, 10%, 15%, 20%, 30%, 40%, 50%, 60%, 70%, 75%, 80%, 90%, 95%, etc.

In some embodiments, the donor cell and recipient express different specific isoforms of one or more polypeptides, wherein the different specific isoforms differ by variation in 1, 2, 3, 4, 5, 6, 7, 8, 9, 10 or more amino acid residues in the polypeptide sequence. In some embodiments, the different specific isoforms differ by variation in 1 amino acid residue in the polypeptide sequences. In some embodiments the different specific isoforms display a single amino acid-polymorphism (SAPs). In some embodiments, the different specific isoforms result from single nucleotide polymorphisms (SNPs). In some embodiments, the different specific isoforms result from frame shifts. In some embodiments, the different specific isoforms result from splice variants. In some embodiments, the specific isoform expressed by donor cells contains 1, 2, 3, 4, 5, 6, 7, 8, 9, 10 or more additional amino acid residues in the polypeptide sequence compared to the recipient. In some embodiments, the specific isoform expressed by the recipient contains 1, 2, 3, 4, 5, 6, 7, 8, 9, 10 or more additional amino acid residues in the polypeptide sequence compared to the donor cells. In some embodiments, the SNPs and/or SAPs do not have functional consequences on the polypeptide. In some embodiments, the SNPs and/or SAPs do not significantly affect the functions of the polypeptide. In some embodiments, the SNPs and/or SAPs do not affect the normal protein expression profile of the donor cells.

In some embodiments, one or more of the polypeptides comprise alpha-1-antitrypsin, wherein the specific isoforms wherein the specific isoforms of alpha-1-antitrypsin differ by variation in 1, 2, 3, 4, 5, 6, 7, 8, 9, 10 or more amino acid residues in the polypeptide sequence. In some embodiments, the different specific isoforms of alpha-1-antitrypsin differ by variation in 1 amino acid residue in the polypeptide sequences. In some embodiments, the specific isoforms of alpha-1-antitrypsin comprise the sequences DTEEEDFHVDQVTTVK (SEQ ID NO:30) and DTEEEDFHVDQATTVK (SEQ ID NO:31). In some embodiments, the specific isoform of alpha-1-antitrypsin expressed by donor cells comprises the sequence DTEEEDFHVDQVTTVK (SEQ ID NO:30) and the specific isoform of alpha-1-antitrypsin expressed by the recipient comprises the sequence DTEEEDFHVDQATTVK (SEQ ID NO: 31). In some embodiments, the specific isoform of alpha-1-antitrypsin expressed by recipient comprises the sequence DTEEEDFHVDQVTTVK (SEQ ID NO:30) and the specific isoform of alpha-1-antitrypsin expressed by donor cells comprises the sequence DTEEEDFHVDQATTVK (SEQ ID NO:31). In some embodiments, the specific isoform of alpha-1-antitrypsin expressed by recipient comprises the sequence DTEEEDFHVDQVTTVK (SEQ ID NO:30) and the specific isoform of alpha-1-antitrypsin expressed by the donor cells comprises a sequence containing a variation in 1, 2, 3, 4, or 5 amino acid residues from DTEEEDFHVDQVTTVK (SEQ ID NO:30). In some embodiments, the specific isoform of alpha-1-antitrypsin expressed by recipient comprises the sequence DTEEEDFHVDQATTVK (SEQ ID NO:31) and the specific isoform of alpha-1-antitrypsin expressed by the donor cells comprises a sequence containing a variation in 1, 2, 3, 4, or 5 amino acid residues from DTEEEDFHVDQATTVK (SEQ ID NO:31).

In some embodiments, one or more of the polypeptides comprise vitamin D binding protein (DBP), wherein the specific isoforms wherein the specific isoforms of DBP differ by variation in 1, 2, 3, 4, 5, 6, 7, 8, 9, 10 or more amino acid residues in the polypeptide sequence. In some embodiments, the different specific isoforms of DBP differ by variation in 1 amino acid residue in the polypeptide sequences. In some embodiments, the specific isoforms of DBP comprise the sequences LPDATPTELAK (SEQ ID NO:3) and LPAATPTELAK (SEQ ID NO:42). In some embodiments, the specific isoform of DBP expressed by donor cells comprises the sequence LPDATPTELAK (SEQ ID NO:3) and the specific isoform of DBP expressed by the recipient comprises the sequence LPAATPTELAK (SEQ ID NO:42). In some embodiments, the specific isoform of DBP expressed by recipient comprises the sequence LPDATPTELAK (SEQ ID NO:3) and the specific isoform of DBP expressed by donor cells comprises the sequence LPAATPTELAK (SEQ ID NO:42). In some embodiments, the specific isoform of DBP expressed by recipient comprises the sequence LPDATPTELAK (SEQ ID NO:3) and the specific isoform of DBP expressed by donor cells comprises a sequence containing a variation in 1, 2, 3, 4, or 5 amino acid residues from LPDATPTELAK (SEQ ID NO:3). In some embodiments, the specific isoform of DBP expressed by recipient comprises the sequence LPAATPTELAK (SEQ ID NO:42) and the specific isoform of DBP expressed by donor cells comprises a sequence containing a variation in 1, 2, 3, 4, or 5 amino acid residues from LPAATPTELAK (SEQ ID NO:42).

In some embodiments, polypeptides are selected to distinguish donor and recipient hepatocytes in an individual, wherein the polypeptide is one of more of coagulation factor V, vitamin D binding protein, transthyretin, apolipoprotein E, beta-2-glycoprotein 1, or carboxypeptidase N subunit 2. In some embodiments, the polypeptide is encoded by one of the following genes: F5, GC, HEL111, APOE, APOH, or CPN2.

In some embodiments, a panel of polypeptides are selected to distinguish donor and recipient hepatocytes in an individual, wherein the panel comprises two or more of coagulation factor V, vitamin D binding protein, transthyretin, apolipoprotein E, beta-2-glycoprotein 1, and carboxypeptidase N subunit 2. In some embodiments, the panel comprises three or more, four or more, five or more of coagulation factor V, vitamin D binding protein, transthyretin, apolipoprotein E, beta-2-glycoprotein, and carboxypeptidase N subunit 2. In some embodiments, the panel comprises coagulation factor V, vitamin D binding protein, transthyretin, apolipoprotein E, beta-2-glycoprotein, and carboxypeptidase N subunit 2. In some embodiments, the panel comprises two or more polypeptides encoded by the following genes: F5, GC, HEL111, APOE, APOH, and CPN2. In some embodiments, the panel comprises three or more, four or more, or five or more polypeptides encoded by the following genes: F5, GC, HEL111, APOE, APOH, and CPN2. In some embodiments, the panel comprises polypeptides encoded by the following genes: F5, GC, HEL111, APOE, APOH, and CPN2.

In some embodiments, one or more of the polypeptides comprise coagulation factor V, wherein the specific isoforms of coagulation factor V differ by variation in 1, 2, 3, 4, 5, 6, 7, 8, 9, 10 or more amino acid residues in the polypeptide sequence. In some embodiments, the different specific isoforms of coagulation factor V differ by variation in 1 amino acid residue in the polypeptide sequences. In some embodiments, the specific isoforms of coagulation factor V comprise the sequences LLSLGAGEFK (SEQ ID NO:1) and LLSLGAGEFR (SEQ ID NO:2). In some embodiments, the specific isoform of coagulation factor V expressed by donor cells comprises the sequence LLSLGAGEFK (SEQ ID NO:1) and the specific isoform of coagulation factor V expressed by the recipient comprises the sequence LLSLGAGEFR (SEQ ID NO:2). In some embodiments, the specific isoform of coagulation factor V expressed by recipient comprises the sequence LLSLGAGEFK (SEQ ID NO: 1) and the specific isoform of coagulation factor V expressed by donor cells comprises the sequence LLSLGAGEFR (SEQ ID NO:2). In some embodiments, the specific isoform of coagulation factor V expressed by recipient comprises a sequence containing a variation in 0, 1, 2, or 3 amino acid residues from the sequence LLSLGAGEFK (SEQ ID NO:1) and the specific isoform of coagulation factor V expressed by the donor cells comprises a sequence containing a variation in 0, 1, 2, or 3 amino acid residues from LLSLGAGEFR (SEQ ID NO: 2). In some embodiments, the specific isoform of coagulation factor V expressed by recipient comprises a sequence containing a variation in 0, 1, 2, or 3 amino acid residues from the sequence LLSLGAGEFR (SEQ ID NO:2) and the specific isoform of coagulation factor V expressed by the donor cells comprises a sequence containing a variation in 0, 1, 2, or 3 amino acid residues from LLSLGAGEFK (SEQ ID NO:1). In some embodiments, the specific isoform of coagulation factor V expressed by recipient comprises a sequence of SEQ ID NO: 1, and the specific isoform of coagulation factor V expressed by the donor cells comprises a sequence containing a variation in 1, 2, 3, 4, or 5 amino acid residues from SEQ ID NO: 1. In some embodiments, the specific isoform of coagulation factor V expressed by recipient comprises a sequence of SEQ ID NO:2, and the specific isoform of coagulation factor V expressed by the donor cells comprises a sequence containing a variation in 1, 2, 3, 4, or 5 amino acid residues from SEQ ID NO:2.

In some embodiments, one or more of the polypeptides comprise vitamin D binding protein, wherein the specific isoforms of vitamin D binding protein differ by variation in 1, 2, 3, 4, 5, 6, 7, 8, 9, 10 or more amino acid residues in the polypeptide sequence. In some embodiments, the different specific isoforms of vitamin D binding protein differ by variation in 1 amino acid residue in the polypeptide sequences. In some embodiments, the specific isoforms of vitamin D binding protein comprise the sequences LPDATPTELAK (SEQ ID NO: 3) and LPEATPTELAK (SEQ ID NO:4). In some embodiments, the specific isoform of vitamin D binding protein expressed by donor cells comprises the sequence LPDATPTELAK (SEQ ID NO:3) and the specific isoform of vitamin D binding protein expressed by the recipient comprises the sequence LPEATPTELAK (SEQ ID NO:4). In some embodiments, the specific isoform of vitamin D binding protein expressed by recipient comprises the sequence LPDATPTELAK (SEQ ID NO:3) and the specific isoform of vitamin D binding protein expressed by donor cells comprises the sequence LPEATPTELAK (SEQ ID NO:4). In some embodiments, the specific isoform of vitamin D binding protein expressed by recipient comprises a sequence containing a variation in 0, 1, 2, or 3 amino acid residues from the sequence LPDATPTELAK (SEQ ID NO:3) and the specific isoform of vitamin D binding protein expressed by the donor cells comprises a sequence containing a variation in 0, 1, 2, or 3 amino acid residues from sequence LPEATPTELAK (SEQ ID NO:4). In some embodiments, the specific isoform of vitamin D binding protein expressed by recipient comprises a sequence containing a variation in 0, 1, 2, or 3 amino acid residues from the sequence LPEATPTELAK (SEQ ID NO:4) and the specific isoform of vitamin D binding protein expressed by the donor cells comprises a sequence containing a variation in 0, 1, 2, or 3 amino acid residues from LPDATPTELAK (SEQ ID NO:3). In some embodiments, the specific isoform of vitamin D binding protein expressed by recipient comprises a sequence of SEQ ID NO:3, and the specific isoform of vitamin D binding protein expressed by the donor cells comprises a sequence containing a variation in 1, 2, 3, 4, or 5 amino acid residues from SEQ ID NO:3. In some embodiments, the specific isoform of vitamin D binding protein expressed by recipient comprises a sequence of SEQ ID NO:4, and the specific isoform of vitamin D binding protein expressed by the donor cells comprises a sequence containing a variation in 1, 2, 3, 4, or 5 amino acid residues from SEQ ID NO:4.

In some embodiments, one or more of the polypeptides comprise transthyretin, wherein the specific isoforms of transthyretin differ by variation in 1, 2, 3, 4, 5, 6, 7, 8, 9, 10 or more amino acid residues in the polypeptide sequence. In some embodiments, the different specific isoforms of transthyretin differ by variation in 1 amino acid residue in the polypeptide sequences. In some embodiments, the specific isoforms of transthyretin comprise the sequences AADDTWEPFSSGK (SEQ ID NO:5) and AADDTWEPFASVK (SEQ ID NO:6). In some embodiments, the specific isoform of transthyretin expressed by donor cells comprises the sequence AADDTWEPFSSGK (SEQ ID NO:5) and the specific isoform of transthyretin expressed by the recipient comprises the sequence AADDTWEPFASVK (SEQ ID NO:6). In some embodiments, the specific isoform of transthyretin expressed by recipient comprises the sequence AADDTWEPFSSGK (SEQ ID NO: 5) and the specific isoform of transthyretin expressed by donor cells comprises the sequence AADDTWEPFASVK (SEQ ID NO:6). In some embodiments, the specific isoform of transthyretin expressed by recipient comprises a sequence containing a variation in 0, 1, 2, or 3 amino acid residues from the sequence AADDTWEPFSSGK (SEQ ID NO:5) and the specific isoform of transthyretin expressed by the donor cells comprises a sequence containing a variation in 0, 1, 2, or 3 amino acid residues from AADDTWEPFASVK (SEQ ID NO: 6). In some embodiments, the specific isoform of transthyretin expressed by recipient comprises a sequence containing a variation in 0, 1, 2, or 3 amino acid residues from the sequence AADDTWEPFASVK (SEQ ID NO:6) and the specific isoform of transthyretin expressed by the donor cells comprises a sequence containing a variation in 0, 1, 2, or 3 amino acid residues from AADDTWEPFSSGK (SEQ ID NO:5). In some embodiments, the specific isoform of transthyretin expressed by recipient comprises a sequence of SEQ ID NO: 5, and the specific isoform of transthyretin expressed by the donor cells comprises a sequence containing a variation in 1, 2, 3, 4, or 5 amino acid residues from SEQ ID NO:5. In some embodiments, the specific isoform of transthyretin expressed by recipient comprises a sequence of SEQ ID NO:6, and the specific isoform of transthyretin expressed by the donor cells comprises a sequence containing a variation in 1, 2, 3, 4, or 5 amino acid residues from SEQ ID NO:6.

In some embodiments, one or more of the polypeptides comprise apolipoprotein E, wherein the specific isoforms of apolipoprotein E differ by variation in 1, 2, 3, 4, 5, 6, 7, 8, 9, 10 or more amino acid residues in the polypeptide sequence. In some embodiments, the different specific isoforms of apolipoprotein E differ by variation in 1 amino acid residue in the polypeptide sequences. In some embodiments, the isoforms of apolipoprotein E are encoded by alleles 2, 3, or 4 of the ApoE gene. In some embodiments, the specific isoforms of apolipoprotein E comprise the sequences LGADMEDVCGR (SEQ ID NO:7) and LGADMEDVR (SEQ ID NO:8). In some embodiments, the specific isoform of apolipoprotein E expressed by donor cells comprises the sequence LGADMEDVCGR (SEQ ID NO: 7) and the specific isoform of apolipoprotein E expressed by the recipient comprises the sequence LGADMEDVR (SEQ ID NO:8). In some embodiments, the specific isoform of apolipoprotein E expressed by recipient comprises the sequence LGADMEDVCGR (SEQ ID NO: 7) and the specific isoform of apolipoprotein E expressed by donor cells comprises the sequence LGADMEDVR (SEQ ID NO:8). In some embodiments, the specific isoform of apolipoprotein E expressed by recipient comprises a sequence containing a variation in 0, 1, 2, or 3 amino acid residues from the sequence LGADMEDVCGR (SEQ ID NO:7) and the specific isoform of apolipoprotein E expressed by the donor cells comprises a sequence containing a variation in 0, 1, 2, or 3 amino acid residues from LGADMEDVR (SEQ ID NO: 8). In some embodiments, the specific isoform of apolipoprotein E expressed by recipient comprises a sequence containing a variation in 0, 1, 2, or 3 amino acid residues from the sequence LGADMEDVR (SEQ ID NO:8) and the specific isoform of apolipoprotein E expressed by the donor cells comprises a sequence containing a variation in 0, 1, 2, or 3 amino acid residues from LGADMEDVCGR (SEQ ID NO:7). In some embodiments, the specific isoform of APOE expressed by recipient comprises a sequence of SEQ ID NO:7, and the specific isoform of APOE expressed by the donor cells comprises a sequence containing a variation in 1, 2, 3, 4, or 5 amino acid residues from SEQ ID NO:7. In some embodiments, the specific isoform of APOE expressed by recipient comprises a sequence of SEQ ID NO:8, and the specific isoform of APOE expressed by the donor cells comprises a sequence containing a variation in 1, 2, 3, 4, or 5 amino acid residues from SEQ ID NO:8.

In some embodiments, the specific isoforms of apolipoprotein E comprise the sequences LAVYQAGAR (SEQ ID NO:9) and CLAVYQAGAR (SEQ ID NO:10). In some embodiments, the specific isoform of apolipoprotein E expressed by donor cells comprises the sequence LAVYQAGAR (SEQ ID NO:9) and the specific isoform of apolipoprotein E expressed by the recipient comprises the sequence CLAVYQAGAR (SEQ ID NO:10). In some embodiments, the specific isoform of apolipoprotein E expressed by recipient comprises the sequence LAVYQAGAR (SEQ ID NO:9) and the specific isoform of apolipoprotein E expressed by donor cells comprises the sequence CLAVYQAGAR (SEQ ID NO: 10). In some embodiments, the specific isoform of apolipoprotein E expressed by recipient comprises a sequence containing a variation in 0, 1, 2, or 3 amino acid residues from the sequence LAVYQAGAR (SEQ ID NO:9) and the specific isoform of apolipoprotein E expressed by the donor cells comprises a sequence containing a variation in 0, 1, 2, or 3 amino acid residues from CLAVYQAGAR (SEQ ID NO:10). In some embodiments, the specific isoform of apolipoprotein E expressed by recipient comprises a sequence containing a variation in 0, 1, 2, or 3 amino acid residues from the sequence CLAVYQAGAR (SEQ ID NO: 10) and the specific isoform of apolipoprotein E expressed by the donor cells comprises a sequence containing a variation in 0, 1, 2, or 3 amino acid residues from LAVYQAGAR (SEQ ID NO:9). In some embodiments, the specific isoform of APOE expressed by recipient comprises a sequence of SEQ ID NO:9, and the specific isoform of APOE expressed by the donor cells comprises a sequence containing a variation in 1, 2, 3, 4, or 5 amino acid residues from SEQ ID NO:9. In some embodiments, the specific isoform of APOE expressed by recipient comprises a sequence of SEQ ID NO: 10, and the specific isoform of APOE expressed by the donor cells comprises a sequence containing a variation in 1, 2, 3, 4, or 5 amino acid residues from SEQ ID NO:10.

In some embodiments, one or more of the polypeptides comprise beta-2-glycoprotein 1 (APOH), wherein the specific isoforms of beta-2-glycoprotein 1 differ by variation in 1, 2, 3, 4, 5, 6, 7, 8, 9, 10 or more amino acid residues in the polypeptide sequence. In some embodiments, the different specific isoforms of beta-2-glycoprotein 1 differ by variation in 1 amino acid residue in the polypeptide sequences. In some embodiments, the specific isoforms of beta-2-glycoprotein 1 comprise the sequences EHSSLAFWK (SEQ ID NO:11) and EHSSLAFSK (SEQ ID NO:12). In some embodiments, the specific isoform of beta-2-glycoprotein 1 expressed by donor cells comprises the sequence EHSSLAFWK (SEQ ID NO: 11) and the specific isoform of beta-2-glycoprotein 1 expressed by the recipient comprises the sequence EHSSLAFSK (SEQ ID NO:12). In some embodiments, the specific isoform of beta-2-glycoprotein 1 expressed by recipient comprises the sequence EHSSLAFWK (SEQ ID NO:11) and the specific isoform of beta-2-glycoprotein 1 expressed by donor cells comprises the sequence EHSSLAFSK (SEQ ID NO:12). In some embodiments, the specific isoform of beta-2-glycoprotein 1 expressed by recipient comprises a sequence containing a variation in 0, 1, 2, or 3 amino acid residues from the sequence EHSSLAFWK (SEQ ID NO:11) and the specific isoform of beta-2-glycoprotein 1 expressed by the donor cells comprises a sequence containing a variation in 0, 1, 2, or 3 amino acid residues from EHSSLAFSK (SEQ ID NO:12). In some embodiments, the specific isoform of beta-2-glycoprotein 1 expressed by recipient comprises a sequence containing a variation in 0, 1, 2, or 3 amino acid residues from the sequence EHSSLAFSK (SEQ ID NO:12) and the specific isoform of beta-2-glycoprotein 1 expressed by the donor cells comprises a sequence containing a variation in 0, 1, 2, or 3 amino acid residues from EHSSLAFWK (SEQ ID NO: 11). In some embodiments, the specific isoform of beta-2-glycoprotein 1 expressed by recipient comprises a sequence of SEQ ID NO:11, and the specific isoform of beta-2-glycoprotein 1 expressed by the donor cells comprises a sequence containing a variation in 1, 2, 3, 4, or 5 amino acid residues from SEQ ID NO:11. In some embodiments, the specific isoform of beta-2-glycoprotein 1 expressed by recipient comprises a sequence of SEQ ID NO: 12, and the specific isoform of beta-2-glycoprotein 1 expressed by the donor cells comprises a sequence containing a variation in 1, 2, 3, 4, or 5 amino acid residues from SEQ ID NO: 12.

In some embodiments, one or more of the polypeptides comprise carboxypeptidase N subunit 2, wherein the specific isoforms of carboxypeptidase N subunit 2 differ by variation in 1, 2, 3, 4, 5, 6, 7, 8, 9, 10 or more amino acid residues in the polypeptide sequence. In some embodiments, the different specific isoforms of carboxypeptidase N subunit 2 differ by variation in 1 amino acid residue in the polypeptide sequences. In some embodiments, the specific isoforms of carboxypeptidase N subunit 2 comprise the sequences LTVSIEAR (SEQ ID NO: 13) and LTMSIEAR (SEQ ID NO:14). In some embodiments, the specific isoform of carboxypeptidase N subunit 2 expressed by donor cells comprises the sequence LTVSIEAR (SEQ ID NO:13) and the specific isoform of carboxypeptidase N subunit 2 expressed by the recipient comprises the sequence LTMSIEAR (SEQ ID NO:14). In some embodiments, the specific isoform of carboxypeptidase N subunit 2 expressed by recipient comprises the sequence LTVSIEAR (SEQ ID NO:13) and the specific isoform of carboxypeptidase N subunit 2 expressed by donor cells comprises the sequence LTMSIEAR (SEQ ID NO:14). In some embodiments, the specific isoform of carboxypeptidase N subunit 2 expressed by recipient comprises a sequence containing a variation in 0, 1, 2, or 3 amino acid residues from the sequence LTVSIEAR (SEQ ID NO: 13) and the specific isoform of carboxypeptidase N subunit 2 expressed by the donor cells comprises a sequence containing a variation in 0, 1, 2, or 3 amino acid residues from LTMSIEAR (SEQ ID NO:14). In some embodiments, the specific isoform of carboxypeptidase N subunit 2 expressed by recipient comprises a sequence containing a variation in 0, 1, 2, or 3 amino acid residues from the sequence LTMSIEAR (SEQ ID NO:14) and the specific isoform of carboxypeptidase N subunit 2 expressed by the donor cells comprises a sequence containing a variation in 0, 1, 2, or 3 amino acid residues from LTVSIEAR (SEQ ID NO:13). In some embodiments, the specific isoform of carboxypeptidase N subunit 2 expressed by recipient comprises a sequence of SEQ ID NO: 13, and the specific isoform of carboxypeptidase N subunit 2 expressed by the donor cells comprises a sequence containing a variation in 1, 2, 3, 4, or 5 amino acid residues from SEQ ID NO:13. In some embodiments, the specific isoform of carboxypeptidase N subunit 2 expressed by recipient comprises a sequence of SEQ ID NO: 14, and the specific isoform of carboxypeptidase N subunit 2 expressed by the donor cells comprises a sequence containing a variation in 1, 2, 3, 4, or 5 amino acid residues from SEQ ID NO:14.

In some embodiments, polypeptides are selected to distinguish donor and recipient B cells (e.g., plasma cells) in an individual, wherein the polypeptide is one of more of IGHG1, IGHG2, IGHA1, IGHM, IGKC, and IGLC2, such as one of more of IGHG1, IGHG2, IGHA1, IGHM, and IGKC. In some embodiments, the panel of polypeptides comprises two or more (e.g., 2, 3, 4, or 5) of IGHG1, IGHG2, IGHA1, IGHM, and IGKC, such as all of IGHG1, IGHG2, IGHA1, IGHM, and IGKC. In some embodiments, the polypeptide is encoded by one of the following genes: IGHG1, IGHG2, IGHA1, IGHM, or IGKC.

In some embodiments, a panel of polypeptides are selected to distinguish donor and recipient B cells (e.g., plasma cells) in an individual, wherein the panel comprises two or more (e.g., 2, 3, 4, 5, or 6) of IGHG1, IGHG2, IGHA1, IGHM, IGKC, and IGLC2, such as two or more (e.g., 2, 3, 4, or 5) of IGHG1, IGHG2, IGHA1, IGHM, and IGKC. In some embodiments, the panel comprises two or more polypeptides encoded by two or more (e.g., 2, 3, 4, 5, or 6) of IGHG1, IGHG2, IGHA1, IGHM, IGKC, or IGLC2, such as encoded by two or more (e.g., 2, 3, 4, or 5) of IGHG1, IGHG2, IGHA1, IGHM, and IGKC. In some embodiments, the panel comprises polypeptides encoded by the following genes: IGHG1, IGHG2, IGHA1, IGHM, and IGKC.

In some embodiments, one or more of the polypeptides comprise IGHG1, wherein the specific isoforms of IGHG1 differ by variation in 1, 2, 3, 4, 5, 6, 7, 8, 9, 10 or more amino acid residues in the polypeptide sequence. In some embodiments, the different specific isoforms of IGHG1 differ by variation in 1 amino acid residue in the polypeptide sequences. In some embodiments, the specific isoforms of IGHG1 comprise the sequence of one or more of SEQ ID NOs: 53-56. In some embodiments, the specific isoform of IGHG1 expressed by donor cells comprises the sequence of SEQ ID NO:53 or 55, and the specific isoform of IGHG1 expressed by the recipient comprises the sequence of SEQ ID NO:54 or 56. In some embodiments, the specific isoform of IGHG1 expressed by donor cells comprises the sequence of SEQ ID NO:54 or 56, and the specific isoform of IGHG1 expressed by the recipient comprises the sequence of SEQ ID NO:53 or 55. In some embodiments, the specific isoform of IGHG1 expressed by recipient comprises the sequence of SEQ ID NO:53 or 55, and the specific isoform of IGHG1 expressed by the donor cells comprises a sequence containing a variation in 1, 2, 3, 4, or 5 amino acid residues from SEQ ID NO:53 or 55. In some embodiments, the specific isoform of IGHG1 expressed by recipient comprises the sequence of SEQ ID NO:54 or 56, and the specific isoform of IGHG1 expressed by the donor cells comprises a sequence containing a variation in 1, 2, 3, 4, or 5 amino acid residues from SEQ ID NO:54 or 56. In some embodiments, the specific isoform of IGHG1 expressed by recipient comprises a sequence containing a variation in 0, 1, 2, or 3 amino acid residues from the sequence SEQ ID NO:54 or 56, and the specific isoform of IGHG1 expressed by the donor cells comprises a sequence containing a variation in 0, 1, 2, or 3 amino acid residues from SEQ ID NO:53 or 55. In some embodiments, the specific isoform of IGHG1 expressed by recipient comprises a sequence containing a variation in 0, 1, 2, or 3 amino acid residues from the sequence SEQ ID NO:53 or 55, and the specific isoform of IGHG1 expressed by the donor cells comprises a sequence containing a variation in 0, 1, 2, or 3 amino acid residues from SEQ ID NO:54 or 56. In some embodiments, the specific isoforms of IGHG1 comprise the sequence of SEQ ID NO:57 and/or SEQ ID NO:58. In some embodiments, the specific isoform of IGHG1 expressed by donor cells comprises the sequence of SEQ ID NO:57, and the specific isoform of IGHG1 expressed by the recipient comprises the sequence of SEQ ID NO: 58. In some embodiments, the specific isoform of IGHG1 expressed by donor cells comprises the sequence of SEQ ID NO:58, and the specific isoform of IGHG1 expressed by the recipient comprises the sequence of SEQ ID NO:57. In some embodiments, the specific isoform of IGHG1 expressed by recipient comprises the sequence of SEQ ID NO:57, and the specific isoform of IGHG1 expressed by the donor cells comprises a sequence containing a variation in 1, 2, 3, 4, or 5 amino acid residues from SEQ ID NO:57. In some embodiments, the specific isoform of IGHG1 expressed by recipient comprises the sequence of SEQ ID NO: 58, and the specific isoform of IGHG1 expressed by the donor cells comprises a sequence containing a variation in 1, 2, 3, 4, or 5 amino acid residues from SEQ ID NO:58. In some embodiments, the specific isoform of IGHG1 expressed by recipient comprises a sequence containing a variation in 0, 1, 2, or 3 amino acid residues from the sequence SEQ ID NO: 57, and the specific isoform of IGHG1 expressed by the donor cells comprises a sequence containing a variation in 0, 1, 2, or 3 amino acid residues from SEQ ID NO:58. In some embodiments, the specific isoform of IGHG1 expressed by recipient comprises a sequence containing a variation in 0, 1, 2, or 3 amino acid residues from the sequence SEQ ID NO: 58, and the specific isoform of IGHG1 expressed by the donor cells comprises a sequence containing a variation in 0, 1, 2, or 3 amino acid residues from SEQ ID NO:57.

In some embodiments, one or more of the polypeptides comprise IGHG2, wherein the specific isoforms of IGHG2 differ by variation in 1, 2, 3, 4, 5, 6, 7, 8, 9, 10 or more amino acid residues in the polypeptide sequence. In some embodiments, the different specific isoforms of IGHG2 differ by variation in 1 amino acid residue in the polypeptide sequences. In some embodiments, the specific isoforms of IGHG2 comprise the sequence of SEQ ID NO: 59 and/or SEQ ID NO:60. In some embodiments, the specific isoform of IGHG2 expressed by donor cells comprises the sequence of SEQ ID NO:59, and the specific isoform of IGHG2 expressed by the recipient comprises the sequence of SEQ ID NO:60. In some embodiments, the specific isoform of IGHG2 expressed by donor cells comprises the sequence of SEQ ID NO:60, and the specific isoform of IGHG2 expressed by the recipient comprises the sequence of SEQ ID NO:59. In some embodiments, the specific isoform of IGHG2 expressed by recipient comprises the sequence of SEQ ID NO:60, and the specific isoform of IGHG2 expressed by the donor cells comprises a sequence containing a variation in 1, 2, 3, 4, or 5 amino acid residues from SEQ ID NO:60. In some embodiments, the specific isoform of IGHG2 expressed by recipient comprises the sequence of SEQ ID NO:59, and the specific isoform of IGHG2 expressed by the donor cells comprises a sequence containing a variation in 1, 2, 3, 4, or 5 amino acid residues from SEQ ID NO:59. In some embodiments, the specific isoform of IGHG2 expressed by recipient comprises a sequence containing a variation in 0, 1, 2, or 3 amino acid residues from the sequence SEQ ID NO:59, and the specific isoform of IGHG2 expressed by the donor cells comprises a sequence containing a variation in 0, 1, 2, or 3 amino acid residues from SEQ ID NO:60. In some embodiments, the specific isoform of IGHG2 expressed by recipient comprises a sequence containing a variation in 0, 1, 2, or 3 amino acid residues from the sequence SEQ ID NO:60, and the specific isoform of IGHG2 expressed by the donor cells comprises a sequence containing a variation in 0, 1, 2, or 3 amino acid residues from SEQ ID NO:59. In some embodiments, the specific isoforms of IGHG2 comprise the sequence of SEQ ID NO:61 and/or SEQ ID NO:62. In some embodiments, the specific isoform of IGHG2 expressed by donor cells comprises the sequence of SEQ ID NO:61, and the specific isoform of IGHG2 expressed by the recipient comprises the sequence of SEQ ID NO:62. In some embodiments, the specific isoform of IGHG2 expressed by donor cells comprises the sequence of SEQ ID NO: 62, and the specific isoform of IGHG2 expressed by the recipient comprises the sequence of SEQ ID NO:61. In some embodiments, the specific isoform of IGHG2 expressed by recipient comprises the sequence of SEQ ID NO:61, and the specific isoform of IGHG2 expressed by the donor cells comprises a sequence containing a variation in 1, 2, 3, 4, or 5 amino acid residues from SEQ ID NO:61. In some embodiments, the specific isoform of IGHG2 expressed by recipient comprises the sequence of SEQ ID NO:62, and the specific isoform of IGHG2 expressed by the donor cells comprises a sequence containing a variation in 1, 2, 3, 4, or 5 amino acid residues from SEQ ID NO:62. In some embodiments, the specific isoform of IGHG2 expressed by recipient comprises a sequence containing a variation in 0, 1, 2, or 3 amino acid residues from the sequence SEQ ID NO:61, and the specific isoform of IGHG2 expressed by the donor cells comprises a sequence containing a variation in 0, 1, 2, or 3 amino acid residues from SEQ ID NO:62. In some embodiments, the specific isoform of IGHG2 expressed by recipient comprises a sequence containing a variation in 0, 1, 2, or 3 amino acid residues from the sequence SEQ ID NO:62, and the specific isoform of IGHG2 expressed by the donor cells comprises a sequence containing a variation in 0, 1, 2, or 3 amino acid residues from SEQ ID NO:61. In some embodiments, the specific isoforms of IGHG2 comprise the sequence of SEQ ID NO:63 and/or SEQ ID NO:64. In some embodiments, the specific isoform of IGHG2 expressed by donor cells comprises the sequence of SEQ ID NO:63, and the specific isoform of IGHG2 expressed by the recipient comprises the sequence of SEQ ID NO:64. In some embodiments, the specific isoform of IGHG2 expressed by donor cells comprises the sequence of SEQ ID NO:64, and the specific isoform of IGHG2 expressed by the recipient comprises the sequence of SEQ ID NO:63. In some embodiments, the specific isoform of IGHG2 expressed by recipient comprises the sequence of SEQ ID NO:63, and the specific isoform of IGHG2 expressed by the donor cells comprises a sequence containing a variation in 1, 2, 3, 4, or 5 amino acid residues from SEQ ID NO: 63. In some embodiments, the specific isoform of IGHG2 expressed by recipient comprises the sequence of SEQ ID NO:64, and the specific isoform of IGHG2 expressed by the donor cells comprises a sequence containing a variation in 1, 2, 3, 4, or 5 amino acid residues from SEQ ID NO:64. In some embodiments, the specific isoform of IGHG2 expressed by recipient comprises a sequence containing a variation in 0, 1, 2, or 3 amino acid residues from the sequence SEQ ID NO:63, and the specific isoform of IGHG2 expressed by the donor cells comprises a sequence containing a variation in 0, 1, 2, or 3 amino acid residues from SEQ ID NO:64. In some embodiments, the specific isoform of IGHG2 expressed by recipient comprises a sequence containing a variation in 0, 1, 2, or 3 amino acid residues from the sequence SEQ ID NO:64, and the specific isoform of IGHG2 expressed by the donor cells comprises a sequence containing a variation in 0, 1, 2, or 3 amino acid residues from SEQ ID NO:63.

In some embodiments, one or more of the polypeptides comprise IGHA1, wherein the specific isoforms of IGHA1 differ by variation in 1, 2, 3, 4, 5, 6, 7, 8, 9, 10 or more amino acid residues in the polypeptide sequence. In some embodiments, the different specific isoforms of IGHA1 differ by variation in 1 amino acid residue in the polypeptide sequences. In some embodiments, the specific isoforms of IGHA1 comprise the sequence of SEQ ID NO: 66 and/or SEQ ID NO:65. In some embodiments, the specific isoform of IGHA1 expressed by donor cells comprises the sequence of SEQ ID NO:65, and the specific isoform of IGHA1 expressed by the recipient comprises the sequence of SEQ ID NO:66. In some embodiments, the specific isoform of IGHA1 expressed by donor cells comprises the sequence of SEQ ID NO:66, and the specific isoform of IGHA1 expressed by the recipient comprises the sequence of SEQ ID NO:65. In some embodiments, the specific isoform of IGHA1 expressed by recipient comprises the sequence of SEQ ID NO:65, and the specific isoform of IGHA1 expressed by the donor cells comprises a sequence containing a variation in 1, 2, 3, 4, or 5 amino acid residues from SEQ ID NO:65. In some embodiments, the specific isoform of IGHA1 expressed by recipient comprises the sequence of SEQ ID NO:66, and the specific isoform of IGHA1 expressed by the donor cells comprises a sequence containing a variation in 1, 2, 3, 4, or 5 amino acid residues from SEQ ID NO:66. In some embodiments, the specific isoform of IGHA1 expressed by recipient comprises a sequence containing a variation in 0, 1, 2, or 3 amino acid residues from the sequence SEQ ID NO:65, and the specific isoform of IGHA1 expressed by the donor cells comprises a sequence containing a variation in 0, 1, 2, or 3 amino acid residues from SEQ ID NO:66. In some embodiments, the specific isoform of IGHA1 expressed by recipient comprises a sequence containing a variation in 0, 1, 2, or 3 amino acid residues from the sequence SEQ ID NO:66, and the specific isoform of IGHA1 expressed by the donor cells comprises a sequence containing a variation in 0, 1, 2, or 3 amino acid residues from SEQ ID NO:65.

In some embodiments, one or more of the polypeptides comprise IGHM, wherein the specific isoforms of IGHM differ by variation in 1, 2, 3, 4, 5, 6, 7, 8, 9, 10 or more amino acid residues in the polypeptide sequence. In some embodiments, the different specific isoforms of IGHM differ by variation in 1 amino acid residue in the polypeptide sequences. In some embodiments, the specific isoforms of IGHM comprise the sequence of SEQ ID NO: 67 and/or SEQ ID NO:68. In some embodiments, the specific isoform of IGHM expressed by donor cells comprises the sequence of SEQ ID NO:67, and the specific isoform of IGHM expressed by the recipient comprises the sequence of SEQ ID NO:68. In some embodiments, the specific isoform of IGHM expressed by donor cells comprises the sequence of SEQ ID NO:68, and the specific isoform of IGHM expressed by the recipient comprises the sequence of SEQ ID NO:67. In some embodiments, the specific isoform of IGHM expressed by recipient comprises the sequence of SEQ ID NO:67, and the specific isoform of IGHM expressed by the donor cells comprises a sequence containing a variation in 1, 2, 3, 4, or 5 amino acid residues from SEQ ID NO:67. In some embodiments, the specific isoform of IGHM expressed by recipient comprises the sequence of SEQ ID NO:68, and the specific isoform of IGHM expressed by the donor cells comprises a sequence containing a variation in 1, 2, 3, 4, or 5 amino acid residues from SEQ ID NO:68. In some embodiments, the specific isoform of IGHM expressed by recipient comprises a sequence containing a variation in 0, 1, 2, or 3 amino acid residues from the sequence SEQ ID NO:67, and the specific isoform of IGHM expressed by the donor cells comprises a sequence containing a variation in 0, 1, 2, or 3 amino acid residues from SEQ ID NO:68. In some embodiments, the specific isoform of IGHM expressed by recipient comprises a sequence containing a variation in 0, 1, 2, or 3 amino acid residues from the sequence SEQ ID NO:68, and the specific isoform of IGHM expressed by the donor cells comprises a sequence containing a variation in 0, 1, 2, or 3 amino acid residues from SEQ ID NO:67. In some embodiments, the specific isoforms of IGHM comprise the sequence of SEQ ID NO:69 and/or SEQ ID NO:70. In some embodiments, the specific isoform of IGHM expressed by donor cells comprises the sequence of SEQ ID NO:69, and the specific isoform of IGHM expressed by the recipient comprises the sequence of SEQ ID NO:70. In some embodiments, the specific isoform of IGHM expressed by donor cells comprises the sequence of SEQ ID NO:70, and the specific isoform of IGHM expressed by the recipient comprises the sequence of SEQ ID NO:69. In some embodiments, the specific isoform of IGHM expressed by recipient comprises the sequence of SEQ ID NO:69, and the specific isoform of IGHM expressed by the donor cells comprises a sequence containing a variation in 1, 2, 3, 4, or 5 amino acid residues from SEQ ID NO: 69. In some embodiments, the specific isoform of IGHM expressed by recipient comprises the sequence of SEQ ID NO:70, and the specific isoform of IGHM expressed by the donor cells comprises a sequence containing a variation in 1, 2, 3, 4, or 5 amino acid residues from SEQ ID NO:70. In some embodiments, the specific isoform of IGHM expressed by recipient comprises a sequence containing a variation in 0, 1, 2, or 3 amino acid residues from the sequence SEQ ID NO:69, and the specific isoform of IGHM expressed by the donor cells comprises a sequence containing a variation in 0, 1, 2, or 3 amino acid residues from SEQ ID NO:70. In some embodiments, the specific isoform of IGHM expressed by recipient comprises a sequence containing a variation in 0, 1, 2, or 3 amino acid residues from the sequence SEQ ID NO:70, and the specific isoform of IGHM expressed by the donor cells comprises a sequence containing a variation in 0, 1, 2, or 3 amino acid residues from SEQ ID NO:69.

In some embodiments, one or more of the polypeptides comprise IGKC, wherein the specific isoforms of IGKC differ by variation in 1, 2, 3, 4, 5, 6, 7, 8, 9, 10 or more amino acid residues in the polypeptide sequence. In some embodiments, the different specific isoforms of IGKC differ by variation in 1 amino acid residue in the polypeptide sequences. In some embodiments, the specific isoforms of IGKC comprise the sequence of SEQ ID NO:71 and/or SEQ ID NO:72. In some embodiments, the specific isoform of IGKC expressed by donor cells comprises the sequence of SEQ ID NO:71, and the specific isoform of IGKC expressed by the recipient comprises the sequence of SEQ ID NO:72. In some embodiments, the specific isoform of IGKC expressed by donor cells comprises the sequence of SEQ ID NO: 72, and the specific isoform of IGKC expressed by the recipient comprises the sequence of SEQ ID NO:71. In some embodiments, the specific isoform of IGKC expressed by recipient comprises the sequence of SEQ ID NO:71, and the specific isoform of IGKC expressed by the donor cells comprises a sequence containing a variation in 1, 2, 3, 4, or 5 amino acid residues from SEQ ID NO:71. In some embodiments, the specific isoform of IGKC expressed by recipient comprises the sequence of SEQ ID NO:72, and the specific isoform of IGKC expressed by the donor cells comprises a sequence containing a variation in 1, 2, 3, 4, or 5 amino acid residues from SEQ ID NO:72. In some embodiments, the specific isoform of IGKC expressed by recipient comprises a sequence containing a variation in 0, 1, 2, or 3 amino acid residues from the sequence SEQ ID NO:71, and the specific isoform of IGKC expressed by the donor cells comprises a sequence containing a variation in 0, 1, 2, or 3 amino acid residues from SEQ ID NO:72. In some embodiments, the specific isoform of IGKC expressed by recipient comprises a sequence containing a variation in 0, 1, 2, or 3 amino acid residues from the sequence SEQ ID NO:72, and the specific isoform of IGKC expressed by the donor cells comprises a sequence containing a variation in 0, 1, 2, or 3 amino acid residues from SEQ ID NO:71.

In some embodiments of the methods of the invention, mass spectrometry suitability is predicted. In some embodiments, mass spectrometry (MS) requires digestion of larger proteins to generate defined peptides in the size range that can be analyzed using this method. In some embodiments, digestion with trypsin, which cleaves on the C-terminal side of lysine residues, is used to generate peptides for MS. In some embodiments, prevalent amino acid variants identified above (e.g., with population frequency above 5%) are digested with trypsin, then subjected to mass spectrometry analysis. In some embodiments, tryptic peptides that span the amino acid variants generate distinct signals, provided that the alternative amino acids in the corresponding variants have different molecular weights. In some embodiments, prediction of tryptic peptides from selected amino acid variants for suitability in MS analysis is performed using Protein Prospector (http://prospector.ucsf.edu/prospector/mshome.htm). The peptide variants that are predicted to be distinguishable are retained on the final list as candidates to identify specific isoforms, such as amino acid variants of plasma proteins synthesized by the cells of interest, e.g., hepatocytes or B cells.

In some embodiments of the methods of the invention, the suitability of variant peptides of polypeptides for mass spectrometry is based on several criteria. In some embodiments, the variant peptides are allelic variants of plasma polypeptides. In some embodiments, the frequency of the variant peptide in a population is greater than about 1% (e.g., greater than about any of 1-5%, such as greater than about 5%). In some embodiments, variant peptides to distinguish donor from recipient have unique sequences. In some embodiments, the variant peptides are stable to chromatography methods used to purify the peptides (e.g., from a plasma sample) prior to mass spectroscopy. In some embodiments, the variant peptides have four or more fragment ions. In some embodiments, the variant peptides do not have a glutamine, a glutamic acid or a proline residue at its N-terminus. In some embodiments, the variant peptides does not contain a methionine reside and/or an asparagine residue. In some embodiments, variant peptides are not selected from polypeptides where missed cleavage of the polypeptide may impact the generation of the peptide (e.g., where different forms of the peptide are generated by missed cleavage). In some embodiments, the variant peptides do not contain cryptic cleavage sites (e.g., cryptic tryptic cleavage sites).

### Processing of Polypeptides and Analysis

### Processing of Polypeptides

In some embodiments, detection of specific isoforms requires detection of sequences with specific polymorphisms. In some embodiments, the specific isoforms of the one or more polypeptides can be detected and/or analyzed from the unprocessed polypeptides. In some embodiments, the specific isoforms of the one or more polypeptides can only be detected and/or analyzed from polypeptides that are processed (such as but not limited to being digested or cleaved). In some embodiments, the one or more polypeptides are processed prior to analysis. In some embodiments, the one or more polypeptides are enzymatically cleaved or chemically cleaved prior to analysis to generate a peptide mixture. In some embodiments, the one or more polypeptides are cleaved with one or more of serine proteases, cysteine proteases, aspartyl proteases or metalloproteases prior to analysis to generate a peptide mixture. In some embodiments, the one or more polypeptides are enzymatically digested using site-specific proteases to generate a peptide mixture. In some embodiments, the one or more polypeptides are cleaved with trypsin prior to analysis to generate a peptide mixture. In some embodiments, prevalent amino acid variant(s) or isoform(s) identified using any of the methods described herein are digested with trypsin in silico. In some embodiments, Protein Prospector (http://prospector.ucsf.edu/prospector/mshome.htm) is used to identify tryptic peptides that are distinguishable, which can serve as candidate peptides for distinguishing individuals, or donor vs. recipient.

In some instances, multi-dimensional polypeptide separation and mass spectrometry may be employed to identify and/or monitor the different specific isoforms of the one or more polypeptides. In some embodiments that can be combined with any of the embodiments herein, the polypeptides are separated prior to analysis. In some embodiments that can be combined with any of the embodiments herein, the peptides in the peptide mixture are separated prior to analysis. In some embodiments, the peptides are separated by chromatography and/or electrophoresis.

Chromatography techniques are well known in the art. These techniques are used to separate organic compounds on the basis of their charge, size, shape, and their solubilities. Chromatography consists of a mobile phase (solvent and the molecules to be separated) and a stationary phase either of paper (in paper chromatography) or glass beads, called resin, (in column chromatography) through which the mobile phase travels. Molecules travel through the stationary phase at different rates because of their chemistry. Types of chromatography that may be employed in the present invention include, but are not limited to, ultra high performance liquid chromatography (UHPLC), high performance liquid chromatography (HPLC), ion exchange chromatography (IEC), and reverse phase chromatography (RP). In some embodiments, chromatography can comprise: adsorption, partition, affinity, gel filtration and molecular sieve, and many specialized techniques for using them including column, paper, and thin-layer chromatography.

Electrophoresis techniques are well known to one of ordinary skill in the art. Electrophoresis is the process of separating molecules on the basis of the molecule's migration through a gel in an applied electric field. In an electric field, a molecule will migrate towards the pole (cathode or anode) that carries a charge opposite to the net charge carried by the molecule. This net charge depends in part on the pH of the medium in which the molecule is migrating. One common electrophoretic procedure is to establish solutions having different pH values at each end of an electric field, with a gradient range of pH in between. At a certain pH, the isoelectric point of a molecule is obtained and the molecule carries no net charge. As the molecule crosses the pH gradient, it reaches an isoelectric point and is thereafter immobile in the electric field. Therefore, this electrophoresis procedure separates molecules according to their different isoelectric points.

In some embodiments that can be combined with any of the embodiments herein, the peptides in the peptide mixture are separated prior to analysis. In some embodiments that can be combined with any of the embodiments herein, the peptides in the peptide mixture are separated prior to digestion. In some embodiments, the peptides in the peptide mixture are separated by multi-dimensional polypeptide separation. In some embodiments, the peptides in the peptide mixture are separated by high performance liquid chromatography (HPLC) or capillary zone electrophoresis (CZE). In some embodiments, multi-dimensional polypeptide separation as contemplated herein may comprise HPLC, ion exchange and/or reversed phase chromatography. In some embodiments, multi-dimensional polypeptide separation may comprise 2D-gel electrophoresis and/or isoelectric focusing electrophoresis. In some examples, multi-dimensional polypeptide separation method is capable of resolving cellular polypeptides such as specific isoforms of one or more polypeptides. As contemplated in the present disclosure, polypeptide separation method as contemplated in the present invention, may be used in combination with techniques such as, but not limited to, chromatography, electrophoresis and mass spectroscopy in the identification and quantification of stem cell derived polypeptides. In some embodiments, multi-dimensional polypeptide separation refers to polypeptide separation comprising at least two separation steps. In some embodiments, multi-dimensional polypeptide separation refers to two or more separation steps that separate polypeptides based on different physical properties of the polypeptide (e.g., a first step that separates based on polypeptide charge and a second step that separates based on polypeptide hydrophobicity).

In some embodiments, the multi-dimensional polypeptide separation may comprise a first dimension separation of polypeptides based on a first physical property. In some embodiments, polypeptides may be separated by pI using isoelectric focusing in the first dimension (see, e.g., Righetti, Laboratory Techniques in Biochemistry and Molecular Biology, 1983). In some embodiments, the first dimension may employ any number of separation techniques including, but not limited to, ion exclusion, ion exchange, normal/reversed phase partition, size exclusion, ligand exchange, liquid/gel phase isoelectric focusing, and adsorption chromatography. In some embodiments, the first dimension be conducted in the liquid phase to enable polypeptides of the separation step to be fed directly into a second liquid phase separation step.

The second dimension of a multi-dimensional polypeptide separation process may separate polypeptides based on a second physical property (i.e., a different property than the first physical property) and is preferably conducted in the liquid phase (e.g., liquid-phase size exclusion). For example, some polypeptides may be separated by hydrophobicity using reversed phase HPLC in the second dimension (see, e.g., Liang et al., 1996; Griffin et al., 1995; Opiteck et al., 1998; Nilsson et al., 1997; Chen et al., 1994 and 1998; Wall et al., 1999; Chong et al., 1999). This method provides for exceptionally fast and reproducible high-resolution separations of polypeptides according to their hydrophobicity and molecular weight. The non-porous (NP) silica packing material can be used in reverse phase (RP) to separations to eliminate problems associated with porosity and low recovery of larger polypeptides, as well as reducing analysis times by as much as one third. Separation efficiency remains high due to the small diameter of the spherical particles, as does the loadability of the reverse phase chromatography columns. However, the second dimension may employ any number of separation techniques. For example, ID SDS PAGE gel may be used. Having the second dimension conducted in the liquid phase facilitates efficient analysis of the separated polypeptides and enables products to be fed directly into additional analysis steps (e.g., directly into mass spectrometry analysis). In some embodiments, proteins can be separated using a PLRP-S stationary phase (300 Å pore size, 3 μm bead size). In some embodiments, proteins can be separated using a C-4 stationary phase (C4-derivatized beads). (see, e.g. Donnelly et al., 2019, Nature Methods 16:587-594). In some embodiments, proteins can be separated using reversed-phase liquid chromatography by superficially porous columns (e.g. Agilent Infinity Lab Poroshell™). In some embodiments the separation steps could be coupled directly to the mass spectrometer for intact mass measurement.

Polypeptides obtained from the second separation step may be mapped using software in order to create a polypeptide pattern analogous to that of the two-dimensional PAGE image based on the two physical properties used in the two separation steps rather than by a second gel-based size separation technique. A polypeptide profile map as contemplated in the present invention, refers to representations of the polypeptide content of a sample. For example, a polypeptide profile map includes 2-dimensional displays of total polypeptide or subsets thereof expressed in a given cell. Polypeptide profile maps may be used for comparing polypeptide expression patterns (e.g., the amount and identity of polypeptides expressed in a sample) between two or more samples. Such comparing allows for the identification of polypeptides that are present in one sample (e.g., a donor sample) and not in another (e.g., recipient cell before transplant), or are over- or under-expressed in one sample compared to the other.

Isoforms may be detected by means of differentiating peptides carrying the respective variations. In some embodiments, the isoform detection methods comprises high-throughput assays. In some embodiments, the isoform detection methods comprises assays using relatively small amount of protein samples. In some embodiments, the specific isoforms are detected by mass spectrometry, ELISA, or surface plasmon resonance. In some embodiments, the specific isoforms are detected by antibody-based affinity assays (including but not limited to proximity extension assays (OLink Proteomics)), RNA aptamers, Edman degradation (including scalable and parallelized Edman degradation). In some embodiments, the specific isoforms are detected by affinity chromatography, protein microarrays, protein-fragment complementation, phage display, or X-ray crystallography. In some embodiments, the specific isoforms are detected by tandem affinity purification-mass spectroscopy (TAP-MS). In some embodiments, the peptide isoforms are characterized using mass spectrometry. For example, the peptides that elute from the chromatography separation are analyzed by mass spectrometry to determine their molecular weight and identity. For this purpose the peptides eluting from the separation can be analyzed simultaneously to determine molecular weight and identity. In some examples, a fraction of the effluent can be used to determine molecular weight by either matrix-assisted laser desorption ionization (MALDI-TOF-MS). In some embodiments, the molecules of the peptide mixture sample or the separated peptide sample can be ionized by compatible ionization methods that do not degrade or fragment the peptides, such as electrospray ionization (ESI), or matrix-assisted laser desorption ionization (MALDI). In some embodiments, the mass-to-charge ratios of the ionized samples are measured with TOF, Orbitrap or FT-ICR. (see e.g., U.S. Pat. No. 6,002,127). In some examples, the remainder of the eluent can be used to determine the identity of the polypeptides via digestion of the polypeptides and analysis of the peptide mass map fingerprints by MALDI-TOF-MS or other methods described herein.

A significant improvement to the 2D electrophoresis/MS fingerprint method is the direct analysis of peptide sequences by tandem mass spectrometry (MS/MS). The key feature of this method is the ability of a tandem mass spectrometer to collect amino acid sequence information from a specific peptide, even if many other peptides are concurrently present in the sample. In some embodiments, fragmentation of samples is carried out by collision-induced dissociation (CID). Here, a peptide ion of interest is isolated from other peptide ions in the mass spectrometer and passed into a collision cell where it undergoes further fragmentation through collision with an inert gas, breaking the peptide randomly at each peptide bond. In some embodiments, fragmentation of peptide samples is carried out by higher energy collision induced dissociation (HCD), or electron transfer dissociation (ETD). ETD can also be coupled to HCD, giving rise to the fragmentation method of electron transfer/higher-energy collision dissociation (EtHCD). The resultant peptide fragment masses create a unique spectrum that can determine the sequence of the parent peptide. In ESI, liquid chromatography is used as a separation technique, producing a steady stream of peptides from the digestion of a complex mixture of polypeptides that are delivered continuously to the mass spectrometer for identification. Both simple and complex polypeptide mixtures can then be analyzed e.g., the polypeptide mixture can be a multiprotein receptor complex, such as the T-cell receptor, or a subcellular domain, such as the membrane fraction of a population of cells. As in protein mass mapping, a search algorithm is then applied, and the masses of every sequence of consecutive amino acids in the database are compared to the experimental fragment masses. Despite the generation of hundreds of thousands of peptide fragments, the probability of a false match is low, and the probability of matching the masses of every amino acid between two different peptides is also low. From the sequence of the peptide, the identity of a polypeptide is determined by correlating the spectrum (such as obtained via CID, HCD, ETD, EtHCD) with the contents of sequence databases. In some embodiments, the separated peptides are characterized by tandem mass spectrometry. For example, the molecules of the peptide mixture sample or the separated peptide sample can be ionized by compatible ionization methods that do not degrade or fragment the peptides, such as electrospray ionization (ESI), or matrix-assisted laser desorption ionization (MALDI). The first spectrum (designated MS1) separates these ions by their mass-to-charge ratio (often given as m/z or m/Q). The mass-to-charge ratios of peptide ions are recorded in an MS1 scan, using a mass analyzer capable of high-resolution and accurate mass. In some embodiments, the mass analyzer is an orbitrap or time-of-flight (TOF) mass analyzer. In some embodiments, ions of a particular m/z-ratio coming from MS1 are selected and then made to split into smaller fragment ions, e.g. by collision-induced dissociation, ion-molecule reaction, or photodissociation. In some embodiments, the selection device comprises a quadrupole or an ion-trap. In some embodiments, the ions are subjected to a fragmentation event in the gas phase such as collision-induced dissociation (CID), higher-energy collisional dissociation (HCD), electron transfer dissociation (ETD), ultraviolet photodissociation (UVPD), or any combinations thereof. These fragments are then measured to give rise to a second spectrum (MS2), which in turn separates the fragments by their m/z-ratio and detects them. The fragmentation step makes it possible to identify and separate ions that would otherwise register very similar m/z-ratios that may not be resolvable in regular mass spectrometry. In some embodiments that can be combined with any of the embodiments herein, the peptides in the peptide mixture are separated by mass to charge ratio prior to analysis in the second spectrometer. In some embodiments, the sequence of the peptides will be determined by fragmentation spectra in a MS2 scan.

Separated polypeptides may be analyzed by mass spectrometry to facilitate the generation of detailed and informative 2D protein maps. The nature of the mass spectrometry technique utilized for analysis in the present invention may include, but is not limited to, orbitrap mass spectrometry, ion trap mass spectrometry, ion trap/time-of-flight mass spectrometry, quadrupole and triple quadrupole mass spectrometry, Fourier Transform (ICR) mass spectrometry, and magnetic sector mass spectrometry. Applications of mass spectrometric methods are well-known to those of skill in the art and are discussed in Methods in Enzymology, 1990.

The quadrupole mass analyzer (QMS) is one type of mass analyzer used in mass spectrometry. It is also known as a transmission quadrupole mass spectrometer, quadrupole mass filter, or quadrupole mass spectrometer. QMS consists of four cylindrical rods, typically set parallel to each other. In some embodiments, the quadrupole is the mass analyzer. In some embodiments, the quadrupole is used for selecting sample ions based on their mass-to-charge ratio (m/z). Ions are separated in a quadrupole based on the stability of their trajectories in the oscillating electric fields that are applied to the rods.

In some embodiments, the mass-to-charge ratios of the intact peptide (such as obtained in MS1 scan) and the series of fragment ions (such as obtained in MS2 scan) are used to confirm the identities of the peptide variants. In some embodiments, the method comprises quantification of the variants by ratiometric comparison to a stable isotope-labeled internal standard of known concentration. Synthetic peptides containing a defined number of isotope labeled residues (such as Carbon-13 and/or nitrogen-15 atoms) will lead to an increase in the mass-to-charge ratio of the peptide without affecting the physicochemical properties of the peptide. In some embodiments, the isotopically labeled peptide will enter the mass spectrometer at the same moment as that from the specimen (i.e. as a spike-in peptide). To enhance the sensitivity of this assay, a quadrupole may be used to limit the mass-range sent to the mass analyzer to perform selected ion monitoring (SIM) scans, over the elution time of the peptide from HPLC without fragmentation. Alternatively, the pair of endogenous sample peptide and spike-in peptide may be isolated by the quadrupole and fragmented prior to mass analysis, with select fragment ions being used for ratiometric quantification, as well as confirming the sequence in a parallel reaction monitoring (PRM) assay. In some embodiments for PRM, the fragmentation spectra for the sample and spike-in peptide are collected separately. In another alternative, a multiple reaction monitoring assay (MRM) can be employed. MRM uses triple quadrupole mass spectrometers coupled to liquid chromatography to enable greater specificity, sensitivity, and quantitation of analytes of interest. In the first quadrupole (Q1), a specific peptide that corresponds to a protein of interest is selected. The peptide is then fragmented in the second quadrupole (Q2) and a filter is applied to allow a specific fragment, also referred to as a transition, to enter into the third quadrupole (Q3) where its intensity is measured. Because of the double selection approach, which improves signal-to-noise and reduces interference, and the multiple measurements per protein, MRM is allows enhanced specificity in isoform measurements.

### Sensitivity of Mass Spectrometry

In some embodiments according to any one of the methods described herein, the method comprises sensitivity to differentiate between isoforms having at least any one of 1%, 2%, 3%, 4%, 5%, 6%, 7%, 8%, 9%, 10%, 12%, 15%, 20% or higher variation in amino acid sequence. In some embodiments according to any one of the methods described herein, the method comprises sensitivity to differentiate between isoforms having at least 1% variation in amino acid sequence. In some embodiments according to any one of the methods described herein, the method comprises sensitivity to differentiate between isoforms comprising 1 amino acid variation in the peptide sample, wherein the peptide length is about any one of: 4, 5, 6, 7, 8, 9, 10, 11, 12, 13, 14, 15, 16, 17, 18, 19, 20, 22, 25, 28, 30, 35, 40, 45 or 50 amino acids. In some embodiments that can be combined any of the methods herein, the method comprises using lower resolution instruments such as triple quadrupole. In some embodiments, the method comprises further steps to improve the dynamic range of mass spectrometry assays. In some embodiments, the method to further improve the dynamic range of mass spectrometry comprises preseparating the protein (such as but not limited to using nanocages for preseparation).

In some aspects, pre-processing of samples are applied to enrich or deplete distinct proteins or protein populations in a sample to increase sensitivity or proteome coverage. This includes technologies such as depletion of high abundant proteins in plasma, serum or CSF samples and approaches that enrich distinct protein populations such as surface modified material e.g. magnetic beads (e.g., ProteoMiner Beads, Seer, etc.). These technologies can be targeted towards a distinct protein such as depletion of the highest abundant protein in plasma (albumin) or might be of a non-targeted nature such as surface modified materials. Pre-processing can also include the enrichment of defined peptides or proteins with an affinity based method to identify exactly these peptides or proteins such as pull-down assays. In some embodiment, targeted mass spectrometry methods is applied to increase sensitivity e.g. MaxQuant Live (Wichmann et al., 2019, Molecular and Cellular Proteomics 18 (5): 982-994).

### Methods of Detecting and Quantitating Transplanted Cells

In some aspects, there is provided a method for detecting a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, there is provided a method for detecting a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein detection of donor-specific isoforms of the one or more polypeptides indicates the presence of donor cells in the recipient.

In some aspects, there is provided a method for quantitating a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates the percentage of donor cells in the recipient. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor cell in the recipient. In some embodiments, the method further comprises obtaining the amount of the donor-specific isoforms of the one or more polypeptides, wherein the amount of the donor-specific isoforms of the one or more polypeptides reflects the quantity of the donor cell in the recipient.

In some aspects, there is provided a method for quantitating a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% (e.g., greater than about any of 1-5%, such as greater than about 5%) of the isoforms of the polypeptide in a population of individuals; and wherein ratio of donor-specific isoforms to recipient-specific isoforms of the one or more polypeptides indicates the percentage of donor cells in the recipient. In some embodiments, the method further comprises obtaining the amount of the recipient-specific isoforms of the one or more polypeptides. In some embodiments, the method further comprises calculating the amount of the donor-specific isoforms of the one or more polypeptides based on the ratio of donor-specific isoforms to recipient-specific isoforms of the one or more polypeptides, wherein the amount of the donor-specific isoforms of the one or more polypeptides reflects the quantity of the donor cell in the recipient. In some embodiments, the method further comprises obtaining the amount of the donor-specific isoforms of the one or more polypeptides, wherein the amount of the donor-specific isoforms of the one or more polypeptides reflects the quantity of the donor cell in the recipient.

In some embodiments, the method unilizes labeled donor-specific isoforms, and/or recipient-specific isoforms of a polypeptide, such as heavy isotope labeled versions thereof, that are spiked-in a sample. Accordingly, in some embodiments, the method comprises labeling, such as using a heavy isotope label, one or more donor-specific isoforms and/or one or more recipient-specific isoforms of a polypeptide. In some embodiments, the method further comprises obtaining a standard curve with known concentrations of the labeled polypeptides, such as isotopically labeled polypeptides. In some embodiments, the method further comprises obtaining the absolute amount of one or more donor-specific isoforms and/or one or more recipient-specific isoforms in a sample based on one or more standard curves. In some embodiments, the quantification of a donor-specific isoform polypeptide is based on a corresponding standard curve generated using a heavy labeled version of the donor-specific isoform polypeptide. In some embodiments, the quantification of a recipient-specific isoform polypeptide is based on a corresponding standard curve generated using a heavy labeled version of the recipient-specific isoform polypeptide.

### Methods of Tracking Transplanted Cells, Monitoring Engraftment and Regenerative Medicine Treatment

In some aspects, there is provided a method for monitoring engraftment and/or function of a donor cell in a recipient, the method comprising: (a) isolating samples from the recipient one or more times after administration of the donor cell to the recipient; and (b) detecting specific isoforms of one or more polypeptides in the samples; wherein the donor cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of donor cells in the recipient.

In some aspects, there is provide a method for monitoring engraftment and/or function of a donor liver cell (e.g., hepatocyte) in a recipient, the method comprising: a) isolating samples from the recipient after administration of the donor liver cell to the recipient; and b) detecting specific isoforms of one or more polypeptides in the samples; wherein the donor liver cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor liver cell in the recipient.

In some aspects, there is provided a method for treating a liver disorder in an individual (e.g., human) in need thereof, comprising administering donor liver cells (e.g., hepatocyte) from a donor individual to the recipient individual in need thereof. In some embodiments, the donor cell expresses a wild-type isoform of the one or more polypeptides described herein, and the recipient expresses a mutant (e.g., disease-related such as disease-causing) isoform of the one or more polypeptides described herein. In some embodiments, the method further comprises monitoring engraftment of the donor liver cells in the recipient individual. In some embodiments, monitoring engraftment of the donor liver cells in the recipient individual is by any of the methods for monitoring engraftment and/or function of donor cells (e.g., donor liver cells) described herein. In some embodiments, there is provided a method for treating a liver disorder in an individual (e.g., human) in need thereof, the method comprising: a) administering donor liver cells (e.g., hepatocyte) from a donor individual to the recipient individual in need thereof; b) isolating samples from the recipient individual after administration of the donor liver cells; and c) detecting specific isoforms of one or more polypeptides in the samples; wherein the donor liver cells and liver cells from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor liver cells in the recipient individual in need thereof. In some embodiments, detecting specific isoforms of the one or more polypeptides is by any of the detecting methods described herein. In some embodiments, the method further comprises selecting the donor individual and/or the recipient individual to be treated. In some embodiments, the donor individual and/or the recipient individual is selected based on the expression of any of the specific isoforms of the one or more polypeptides described herein, such as one or more of coagulation factor V, vitamin D binding protein, transthyretin, apolipoprotein E, beta-2-glycoprotein 1, and carboxypeptidase N subunit 2. In some embodiments, the donor individual and/or the recipient individual is identified by evaluating the level of a serum marker of liver function, e.g., lactate dehydrogenase (LDH), alkaline phosphatase (ALP), aspartate aminotransferase (AST), alanine aminotransferase (ALT), serum bilirubin, albumin, and/or globulins, or based on a test of liver function known in the art.

The term “liver disease” or “liver disorder” applies to many diseases and disorders that cause the liver to function improperly or to cease functioning, and this loss of liver function is indicative of liver disease. Thus, liver function tests are frequently used to diagnose liver disease. Examples of such tests include, but are not limited to: (1) Assays to determine the levels of serum enzymes such as lactate dehydrogenase (LDH), alkaline phosphatase (ALP), aspartate aminotransferase (AST), and alanine aminotransferase (ALT), where an increase in enzyme levels indicates liver disease. One of skill in the art will reasonably understand that these enzyme assays indicate only that the liver has been damaged. They do not assess the liver's ability to function. Other tests can be used to assay a liver's ability to function; (2) Assays to determine serum bilirubin levels. Serum bilirubin levels are reported as total bilirubin and direct bilirubin. Normal values of total serum bilirubin are 0.1-1.0 mg/dL (e.g., about 2-18 mmol/L). Normal values of direct bilirubin are 0.0-0.2 mg/dl (0-4 mmol/L). Increases in serum bilirubin are indicative of liver disease; (3) Assays to determine serum protein levels, for example, albumin and the globulins (e.g., alpha, beta, gamma). Normal values for total serum proteins are 6.0-8.0 g/dl (60-80 g/L). A decrease in serum albumin is indicative of liver disease. An increase in globulin is indicative of liver disease; (4) Other tests include prothrombin time, international normalized ratio, activated clotting time (ACT), partial thromboplastin time (PTT), prothrombin consumption time (PCT), fibrinogen, coagulation factors; alpha-fetoprotein, and alpha-fetoprotein-L3 (percent).

In some embodiments, the liver disorder is any of liver fibrosis, cirrhosis, acute liver failure, fulminant hepatic failure (FHF), or any other degenerative liver disease. In some embodiments, the liver disorder is associated with the loss of liver function and/or the loss or damage of hepatocytes, e.g., of parenchymal liver cells. In some embodiments, the liver disorder is a result of a viral or other infection, an autoimmune disorder, a bile duct obstruction, metabolic disorders, alcohol abuse, primary biliary cirrhosis, NASH, exposure to chemicals, and/or cancer. In some embodiments, the liver disorder comprise liver cancer (e.g., hepatocellular carcinoma (HCC)), primary biliary cirrhosis, autoimmune hepatitis, chronic liver disease, cirrhosis of the liver, hepatitis, viral hepatitis (hepatitis a, hepatitis b, chronic hepatitis b, hepatitis c, chronic hepatitis c, hepatitis d, hepatitis e, hepatitis x), liver failure, jaundice, neonatal jaundice, hepatoma, liver abscess, alcoholic liver disease, hemochromatosis, Wilson's disease, portal hypertension, primary sclerosing cholangitis, sarcoidosis, tapeworms, alveolar hydatid disease, fascioliasis, schistosomiasis, gaucher disease, Zellweger syndrome, alcoholism, food poisoning, pneumococcal pneumonia or Vibrio vulnificus.

In some embodiments, the liver disorder is liver failure. Liver failure occurs when large parts of the liver become damaged and the liver is no longer able to perform its normal physiological function. In some aspects, liver failure can be diagnosed using the above described assays of liver function. In some embodiments, liver failure can be diagnosed (e.g., initially diagnosed) based on a subject's symptoms. Symptoms that are associated with liver failure include, for example, one or more of the following, nausea, loss of appetite, fatigue, diarrhea, jaundice, abnormal/excessive bleeding (e.g., coagulopathy), swollen abdomen, mental disorientation or confusion (e.g., hepatic encephalopathy), sleepiness, and coma. Liver failure can be acute or chronic.

In some aspects, there is provide a method for monitoring engraftment and/or function a donor B cell in a recipient, the method comprising: a) isolating samples from the recipient after administration of the donor B cell to the recipient; and b) detecting specific isoforms of one or more polypeptides in the samples; wherein the donor B cell and the recipient express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor B cell in the recipient.

In some aspects, there is provide a method for treating a B cell disorder in an individual (e.g., human) in need thereof, comprising administering donor B cells from a donor individual to the recipient individual in need thereof. In some embodiments, the donor cell expresses a wild-type isoform of the one or more polypeptides described herein, and the recipient expresses a mutant (e.g., disease-related such as disease-causing) isoform of the one or more polypeptides described herein. In some embodiments, the method further comprises monitoring engraftment of the donor B cells in the recipient individual. In some embodiments, monitoring engraftment of the donor B cells in the recipient individual is by any of the methods for monitoring engraftment and/or function of donor cells (e.g., donor B cells) described herein. In some embodiments, there is provided a method for treating a B cell disorder in an individual (e.g., human) in need thereof, the method comprising: a) administering donor B cells from a donor individual to the recipient individual in need thereof; b) isolating samples from the recipient individual after administration of the donor B cells; and c) detecting specific isoforms of one or more polypeptides in the samples; wherein the donor B cells and cells from the recipient individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment and/or function of the donor B cells in the recipient individual in need thereof. In some embodiments, detecting specific isoforms of the one or more polypeptides is by any of the detecting methods described herein. In some embodiments, the method comprises implantation of the donor bone marrow in the recipient individual in need thereof. In some embodiments, the method further comprises purging the bone marrow of the recipient individual in need thereof before the donor implantation. In some embodiments, the method further comprises selecting the donor individual and/or the recipient individual to be treated. In some embodiments, the donor individual and/or the recipient individual is selected based on the expression of any of the specific isoforms of the one or more polypeptides described herein, such as one or more of IGHG1, IGHG2, IGHA1, IGHM, IGKC, and IGLC2, or one or more of IGHG1, IGHG2, IGHA1, IGHM, and IGKC. In some embodiments, the donor individual and/or the recipient individual is identified by evaluating the expression and/or level of a B cell marker, e.g., one or more of CD19, CD20, BCMA (CD269), CD21, CD22, CD23, CD24, CD40, CD72, CD79a, CD79b, and CD138, such as in a blood sample from the donor individual and/or the recipient individual.

In some embodiments, the B cell disorder is a B cell cancer. B cell cancers include any cancer of B cell origin. In some embodiments, the B cell disorder is a B cell lymphoma, including but is not limited to, diffuse large B cell lymphoma (DLBCL), Burkitt lymphoma, chronic lymphocytic leukemia (CLL), myeloid leukemia (e.g., AML or CML), lymphoid leukemia (e.g., ALL, myelodysplasia), small lymphocytic lymphoma (SLL), follicular lymphoma (FL), mantle cell lymphoma (MCL), marginal zone lymphoma, lymphoplasmacytic lymphoma (Waldenstrom macroglobulinemia), hairy cell leukemia (HCL), primary CNS lymphoma, primary intraocular lymphoma, mucosa-associated-lymphoid tissue B cell lymphoma.

In some embodiments, the B cell disorder is an autoimmune disease. Autoimmune diseases are mediated by autoreactive antibodies, having binding specificity directed against self antigens. Patients suffering from autoimmune diseases typically have high serum titers of autoreactive antibodies, binding for example, to phospholipid, dsDNA, etc. Representative autoimmune diseases that can be treated using the methods and compositions (e.g., donor cell composition) described herein include cold agglutinin disease, systemic lupus erythematosis, rheumatoid arthritis, autoimmune lymphoproliferative disease, multiple sclerosis, psoriasis, and myasthenia gravis, but can also include Hashimoto's thyroiditis, lupus nephritis, dermatomyositis, Sjogren's syndrome, Sydenham's chorea, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, Crohn's disease, Alzheimer's disease, sarcoidosis, ulcerative colitis, erythema multiforme, IgA nephropathy, polyarteritis nodosa, ankylosing spondylitis, Goodpasture's syndrome, thromboangitis ubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, fibrosing alveolitis, Class III autoimmune diseases such as immune-mediated thrombocytopenias, such as acute idiopathic thrombocytopenia purpura and chronic idiopathic thrombocytopeniac purpura, and the like.

In some aspects, there is provided a method for treating an individual in need thereof, the method comprising: a) administering cells from a donor to the individual; b) isolating samples from the individual after administration of the cells from the donor; and c) detecting specific isoforms of one or more polypeptides in the samples; wherein the donor cell and cells from the individual express different specific isoforms of each of the one or more polypeptides, and wherein the specific isoforms comprise greater than about 1% (e.g., greater than about any of 1-5%, such as greater than about 5%) of the isoforms of the polypeptide in a population of individuals; wherein detection of donor-specific isoforms of the one or more polypeptides indicates engraftment of donor cells in the individual.

In some embodiments, additional active agents are administered to the recipient individual in addition to the donor cells.

### Kits

The invention also provides kits comprising components of the methods described herein (e.g., donor cells, such as donor cells expressing any specific isoforms of the one or more polypeptides described herein) and may further comprise instructions for engrafting (e.g., bone marrow implementation), and/or performing said methods to track cell engraftment in a recipient. The kits described herein may further include other materials, including buffers, diluents, filters, needles, syringes, and package inserts with instructions for performing any of the methods described herein; e.g., instructions for tracking and/or quantitating engrafted cells in a recipient.

### Exemplary Embodiments

The invention provides the following enumerated embodiments.

1. A method for detecting a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than 1-5% of the isoforms of the polypeptide in a population of individuals; and wherein detection of donor-specific isoforms of the one or more polypeptides indicates the presence of donor cells in the recipient.

2. A method for detecting a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than 1-5% of the isoforms of the polypeptide in a population of individuals; and wherein detection of donor-specific isoforms of the one or more polypeptides indicates the presence of donor cells in the recipient.

3. A method for quantitating a donor cell in a recipient, the method comprising isolating a sample from the recipient and detecting specific isoforms of one or more polypeptides in the sample; wherein the donor cell and the recipient express different specific isoforms of each of the one or more polypeptides, wherein the specific isoforms comprise greater than 1-5% of the isoforms of the polypeptide in a population of individuals; and wherein ratio of donor-specific isoforms to recipient-specific isoforms of the one or more polypeptides indicates a quantitative measure of the donor cells compared to the recipient cells or a measure of the performance of the donor cells in the recipient.

4. A method for quantitating a donor cell in a recipient, the method comprising detecting specific isoforms of one or more polypeptides in the sample; wherein the donor cell and the recipient express different specific isoforms of each of the one or more polypeptides, wherein the specific isoforms comprise greater than 1-5% of the isoforms of the polypeptide in a population of individuals; and wherein ratio of donor-specific isoforms to recipient-specific isoforms of the one or more polypeptides indicates a quantitative measure of the donor cells compared to the recipient cells or a measure of the performance of the donor cells in the recipient.

5. A method for monitoring engraftment and/or function of a donor cell in a recipient, the method comprising a) isolating samples from the recipient one or more times after administration of the donor cell to the recipient; and b) detecting specific isoforms of one or more polypeptides in the samples; wherein the donor cell and the recipient express different specific isoforms of each of the one or more polypeptides, and wherein the specific isoforms comprise greater than 1-5% of the isoforms of the polypeptide in a population of individuals; wherein detection of donor-specific isoforms of the one or more polypeptides indicates engraftment of donor cells in the recipient.

6. A method for monitoring engraftment and/or function of a donor cell in a recipient, the method comprising detecting specific isoforms of one or more polypeptides in samples from the recipient; wherein the samples had been obtained for one or more times after administration wherein the donor cell and the recipient express different specific isoforms of each of the one or more polypeptides, and wherein the specific isoforms comprise greater than 1-5% of the isoforms of the polypeptide in a population of individuals; wherein detection of donor-specific isoforms of the one or more polypeptides indicates engraftment of donor cells in the recipient.

7. The method of embodiment 5 or 6, wherein the samples are isolated or obtained within about one week, about two weeks, about three weeks, about one month, about two months, about three months, about four months, about five months, about six months, about nine months, about one year, or more than one year after administration of the cells.

8. The method of any one of embodiments 1 to 7, wherein the sample is a plasma sample, a serum sample, a whole blood sample, a lymph sample, a cerebrospinal fluid sample, a urine sample or a liquid or solid biopsy sample.

9. The method of any one of embodiments 1-8, wherein the donor cells are stem cells.

10. The method of embodiment 9, wherein the stem cells are adult stem cells.

11. The method of embodiment 10, wherein the adult stem cells are stromal stem cells, optionally, mesenchymal stromal stem cells, or stem cells derived from tissue, optionally adipose tissue, bone marrow, umbilical cord tissue, liver, pancreas, muscle, heart, kidney, lung, CNS, skin, reproductive tissue, bladder, eye, skeletal, intestinal, or spleen.

12. The method of any one of embodiments 1-8, wherein the donor cells are derived from embryonic stem cells (ESCs) or induced pluripotent stem cells (iPSCs).

13. The method of any one of embodiments 1-8, wherein the donor cells are derived from an organ.

14. The method of embodiment 13, wherein the organ is liver, pancreas, kidney, lung, brain, heart, bladder, spleen, intestines, muscle, eye, skin, blood or marrow.

15. The method of any one of embodiments 1-8, wherein the donor cells are hepatocytes, adipocytes, pancreatic cells, cardiomyocytes, neurons, retinal cells, corneal cells, stromal cells, hematopoietic cells, or skin cells.

16. The method of any one of embodiments 1-8, wherein the donor cells are hepatocyte-like cells, pancreatic progenitor cells, pancreatic beta cells, motor neurons, oligodendrocytes, oligodendrocyte precursor cells, retinal pigment epithelial cells, retinal ganglion cells, retinal progenitor cells, corneal limbal stem cells, corneal endothelial stem cells, stromal stem cells, keratinocytes, cholangiocytes, pericytes, endothelial cells, Kupffer cells, or fibroblasts.

17. The method of any one of embodiments 1-8, wherein the donor cells are synthetic cells.

18. The method of any one of embodiments 1-17, wherein the donor cells are modified.

19. The method of any one of embodiments 1-18, wherein the donor cells are genetically engineered.

20. The method of embodiment 19, wherein the donor cells are genetically engineered to express an isoform of one or more of the specific polypeptides.

21. The method of any one of embodiments 1-20, wherein the one or more specific polypeptides are organ-specific or tissue-specific polypeptides.

22. The method of any one of embodiments 1-21, wherein the one or more specific polypeptides are a panel of organ-specific or tissue-specific polypeptides.

23. The method of any one of embodiments 1-21, wherein the one or more specific polypeptides are ubiquitous polypeptides.

24. The method of any one of embodiments 1-23, wherein the recipient is a mammal.

25. The method of any one of embodiments 1-24, wherein the recipient is a human.

26. The method of any one of embodiments 1-25, wherein the recipient has a disease or disorder treatable by cell engraftment.

27. The method of any one of embodiments 1-26, wherein the specific isoforms are detected by mass spectrometry, ELISA, surface plasmon resonance, affinity-based methods, or polypeptide sequencing-based methods.

28. The method of any one of embodiments 1-27, wherein the one or more polypeptides are enzymatically cleaved prior to analysis to generate a peptide mixture.

29. The method of embodiment 28, wherein the one or more polypeptides are cleaved with one or more enzymes prior to analysis to generate a peptide mixture.

30. The method of embodiment 27 or 28, wherein the one or more polypeptides are cleaved with trypsin prior to analysis to generate a peptide mixture.

31. The method of embodiment 30, wherein the one or more polypeptides are cleaved with trypsin and LysC prior to analysis to generate a peptide mixture.

32. The method of any one of embodiments 28-31, wherein the peptide mixture is separated prior to analysis.

33. The method of embodiment 32, wherein the peptide mixture is separated by high performance liquid chromatography (HPLC) or capillary zone electrophoresis (CZE).

34. The method of any one of embodiments 27-33, wherein the specific isoforms are detected by tandem mass spectrometry.

35. The method of any one of embodiments 27-34, wherein the mass spectrometry includes electrospray ionization (ESI) or matrix-assisted laser desorption ionization (MALDI).

36. The method of any one of embodiments 27-35, wherein the mass spectrometry is tandem mass spectrometry.

37. The method of any one of embodiments 27-36, wherein the mass-to-charge ratios of peptide ions will be recorded in an MS1 scan using a mass analyzer capable of high-resolution and accurate mass.

38. The method of embodiment 37, wherein the mass analyzer is an orbitrap or time-of-flight (TOF) mass analyzer.

39. The method of any one of embodiments 28-38, wherein the sequence of the peptides will be determined by fragmentation spectra.

40. The method of embodiment 39, wherein the ions will be subjected to a fragmentation event in the gas phase.

41. The method of embodiment 40, wherein the ions are subjected to collision-induced dissociation (CID), higher-energy collisional dissociation (HCD), electron transfer dissociation (ETD), ultraviolet photodissociation (UVPD), or a combination thereof.

42. A method for monitoring engraftment of a donor liver cell in a recipient, the method comprising a) isolating samples from the recipient after administration of the donor cell to the recipient; and b) detecting specific isoforms of one or more polypeptides in the samples; wherein the donor cell and the recipient express different specific isoforms of each of the one or more polypeptides, and wherein the specific isoforms comprise greater than 1-5% of the isoforms of the polypeptide in a population of individuals; wherein detection of donor-specific isoforms of the one or more polypeptides indicates engraftment of donor cells in the recipient.

43. The method of embodiment 42, wherein the one or more polypeptides are enzymatically, chemically and/or physically cleaved prior to analysis.

44. The method of embodiment 42 or 43, wherein the specific isoforms are detected by mass spectrometry.

45. The method of embodiment 44, wherein the one or more polypeptides are enzymatically cleaved prior to analysis to generate a peptide mixture.

46. The method of embodiment 45, wherein the one or more polypeptides are cleaved with one or more enzymes prior to analysis to generate a peptide mixture.

47. The method of embodiment 45 or 46, wherein the one or more polypeptides are cleaved with trypsin prior to analysis to generate a peptide mixture.

48. The method of any one of embodiments 45-47 wherein the one or more polypeptides are cleaved with trypsin and LysC prior to analysis to generate a peptide mixture.

49. The method of any one of embodiments 42-48, wherein the one or more polypeptides are alpha-1-antitrypsin, vitamin D binding protein, coagulation factor V, transthyretin, apolipoprotein E, beta-2-glycoprotein, or carboxypeptidase N subunit 2.

50. The method of any one of embodiments 42-48, wherein the one or more polypeptides are a panel of polypeptides.

51. The method of embodiment 50, wherein the panel of polypeptides comprises two, three, four, five, six, seven, eight, nine, ten or more than ten polypeptides.

52. The method of embodiment 50 or 51, wherein the panel of polypeptides comprises two or more of alpha-1-antitrypsin, vitamin D binding protein, coagulation factor V, transthyretin, apolipoprotein E, beta-2-glycoprotein, or carboxypeptidase N subunit 2.

53. The method of any one of embodiments 42-52, wherein the one or more polypeptides is alpha-1-antitrypsin wherein the isoforms comprise the sequences DTEEEDFHVDQVTTVK (SEQ ID NO:30) and DTEEEDFHVDQATTVK (SEQ ID NO: 31).

54. The method of any one of embodiments 42-52, wherein the one or more polypeptides is coagulation factor V wherein the isoforms comprise the sequences LLSLGAGEFK (SEQ ID NO:1) and LLSLGAGEFR (SEQ ID NO:2).

55. The method of any one of embodiments 42-52, wherein the one or more polypeptides is vitamin D binding protein wherein the isoforms comprise the sequences LPDATPTELAK (SEQ ID NO:3) and/or LPEATPTELAK (SEQ ID NO:4) and/or LPAATPTELAK (SEQ ID NO:42).

56. The method of any one of embodiments 42-52, wherein the one or more polypeptides is transthyretin wherein the isoforms comprise the sequences AADDTWEPFSSGK (SEQ ID NO:5) and AADDTWEPFASVK (SEQ ID NO:6).

57. The method of any one of embodiments 42-52, wherein the one or more polypeptides is apolipoprotein E wherein the isoforms comprise the sequences LGADMEDVCGR (SEQ ID NO:7) and LGADMEDVR (SEQ ID NO:8).

58. The method of any one of embodiments 42-52, wherein the one or more polypeptides is apolipoprotein E wherein the isoforms comprise the sequences LAVYQAGAR (SEQ ID NO:9) and CLAVYQAGAR (SEQ ID NO:10).

59. The method of any one of embodiments 42-52, wherein the one or more polypeptides is beta-2-glycoprotein wherein the isoforms comprise the sequences EHSSLAFWK (SEQ ID NO:11) and EHSSLAFSK (SEQ ID NO:12).

60. The method of any one of embodiments 42-52, wherein the one or more polypeptides is carboxypeptidase N subunit 2 wherein the isoforms comprise the sequences LTVSIEAR (SEQ ID NO:13) and LTMSIEAR (SEQ ID NO:14).

61. The method of any one of embodiments 42-60, wherein the specific isoforms are detected by mass spectrometry, ELISA, surface plasmon resonance, affinity-based methods, or polypeptide sequencing-based methods.

62. The method of any one of embodiments 42-61, wherein the one or more polypeptide is enzymatically cleaved prior to analysis to generate a peptide mixture.

63. The method of embodiment 62, wherein the one or more polypeptide is cleaved with trypsin prior to analysis to generate a peptide mixture.

64. The method of embodiment 62 or 63, wherein the peptide mixture is separated prior to analysis.

65. The method of any one of embodiments 62-64, wherein the peptide mixture is separated by high performance liquid chromatography (HPLC) or capillary zone electrophoresis (CZE).

66. The method of any one of embodiments 61-65, wherein the specific isoforms are detected by tandem mass spectrometry.

67. The method of any one of embodiments 61-66, wherein the mass spectrometry includes electrospray ionization (ESI) or matrix-assisted laser desorption ionization (MALDI).

68. The method of any one of embodiments 61-67, wherein the mass spectrometry is tandem mass spectrometry.

69. The method of any one of embodiments 61-68, wherein the mass-to-charge ratios of peptide ions will be recorded in an MS1 scan using a mass analyzer capable of high-resolution and accurate mass.

70. The method of embodiment 69, wherein the mass analyzer is an orbitrap or time-of-flight (TOF) mass analyzer.

71. The method of any one of embodiments 61-70, wherein the sequence of the peptides will be determined by fragmentation spectra in a MS2 scan.

72. The method of embodiment 71, wherein the ions will be subjected to a fragmentation event in the gas phase.

73. The method of embodiment 72, wherein the ions are subjected to collision-induced dissociation (CID), higher-energy collisional dissociation (HCD), electron transfer dissociation (ETD), ultraviolet photodissociation (UVPD), or a combination thereof.

74. A method for treating an individual in need thereof, the method comprising a) administering donor cells from a donor individual to a recipient individual; b) isolating samples from the recipient individual after administration of the donor cells; and c) detecting specific isoforms of one or more polypeptides in the samples; wherein the donor cells and cells from the recipient individual express different specific isoforms of each of the one or more polypeptides, and wherein the specific isoforms comprise greater than 1-5% of the isoforms of the polypeptide in a population of individuals; wherein detection of donor-specific isoforms of the one or more polypeptides indicates engraftment of donor cells in the recipient individual.

75. The method of embodiment 74, wherein the detecting specific isoforms of one or more polypeptides of step c) is by the method of any one of embodiments 1, 2 or 8-41.

76. A method for treating a liver disorder in an individual in need thereof, the method comprising a) administering donor hepatocytes from a donor individual to a recipient individual; b) isolating samples from the recipient individual after administration of the donor hepatocytes; and c) detecting specific isoforms of one or more polypeptides in the samples; wherein the donor hepatocytes and hepatocytes from the recipient individual express different specific isoforms of each of the one or more polypeptides, and wherein the specific isoforms comprise greater than 1-5% of the isoforms of the polypeptide in a population of individuals; wherein detection of donor-specific isoforms of the one or more polypeptides indicates engraftment of donor hepatocytes in the recipient individual.

77. The method of embodiment 76, wherein the detecting specific isoforms of one or more polypeptides of step c) is by the method of any one of embodiments 42-73.

78. A kit for use of the methods of any one of embodiment 1-77.

79. The kit of embodiment 78, wherein the kit further comprises buffers, diluents, filters, needles, syringes for performing the methods of any one of embodiments 1-77.

80. The kit of embodiment 78 or 79, wherein the kit further comprises instructions for performing said methods to detect, quantify, and or monitor cell engraftment in a recipient individual.

### Examples

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### Example 1. Identification of amino acid variants of hepatocyte synthesized proteins

### Introduction

The purpose of the experiment was to identify amino acid variants of plasma proteins synthesized by hepatocytes. There are several criteria for selecting candidate plasma proteins for the purposes of this experiment. First, the plasma proteins should be reasonably abundant in plasma to facilitate detection. The level of detection of individual proteins in plasma is 0.0001%. Second, the population prevalence of the variant alleles (resulting in the protein variants) should be high enough to enable use of the candidate protein variants as a factor to distinguish between cells from different individuals. Generally, a population prevalence of 1-5% or greater for each variant allele being used in detection means that there is a reasonable chance that an individual with that allele will be identified in any population sample. By combining alleles with higher prevalence in the detection regime, the probability of distinguishing between any two individuals increases. A combination of 2 or more alleles should provide a clinically relevant combinatorial probability of distinguishing between most individuals. Third, the peptide on which the variant amino acid is found should be detectable, such as by mass spectrometry. This Example shows a top-down computational method for candidate peptide identification.

### Methods and Results

A) Selecting abundant plasma proteins: Lists of proteins found in plasma are available in the public domain (e.g., Anderson Mol Cell Proteomics 2002). The proteins in these lists were categorized by the cell type they are predominantly synthesized by, such as B lymphocytes and hepatocytes. The list or proteins in plasma predominantly synthesized by hepatocytes were then ordered by their respective concentrations in plasma.

B) Verification of RNA abundance: The plasma protein abundance, as ordered above in step A), was subsequently validated using the Human Protein Atlas (https://www.proteinatlas.org/) which contains abundance of RNA in different tissues. The database was searched for liver-specific proteins, and the corresponding RNA abundance from the Human Protein Atlas was used to verify the relative abundance of the proteins as identified using the protein database.

C) Identification of prevalent amino acid variants: Proteins (as verified in the above combined list from step B)) that were ranked in the top half in the list in terms of abundances (above 50 percentile) were further analyzed for naturally occurring amino acid variants using the UniProt database (https://www.uniprot.org/). For each protein the raw data on the naturally occurring amino acid variants was downloaded and filtered for variants with a population prevalence of greater than 5%. The corresponding allelic frequencies for each protein variant were also determined from the following databases: genomeADbrowser (https://gnomad.broadinstitute.org/), NHLBI Trans-Omics for Precision Medicine (TOPMED, https://www.nhlbiwgs.org/), and ExAC Browser (https://www.re3data.org/repository/r3d100012122).

D) Iterative selection of prevalent variants within abundant plasma proteins: After determining the population frequency of the amino acid variants in the list of proteins from step C) above, 20 variants were selected as follows. Starting with the proteins of highest abundance in plasma, the known variants were examined. If any of the proteins had amino acid variants with population frequency above 5%, that corresponding variant was selected. If a protein had no amino acid variants with population frequency above 5%, the search would move to the protein with the next highest abundance in plasma. The process was repeated until 20 amino acid variants were selected. The process could be continued to generate additional variants with frequencies above 5%. The population frequency threshold at 5% could be modified according to needs and specific applications. For example, population frequency threshold could be preset at 1%, 2%, 5%, 8%, 10%, 15%, 20%, 30%, 50%, 75%, 90%, etc.

E) Mass spectrometry suitability prediction: Mass spectrometry (MS) requires digestion of larger proteins to generate defined peptides in the size range that can be analyzed using this method. Typically, digestion with trypsin, which cleaves on the C-terminal side of lysine residues, is used to generate peptides for MS. Tryptic peptides that span the amino acid variants should generate distinct signals, provided the alternative amino acids in the corresponding peptide variants have different molecular weights. Prediction of tryptic peptides from the selected amino acid variants for suitability in MS analysis was performed using Protein Prospector (http://prospector.ucsf.edu/prospector/mshome.htm). The peptide variants that were predicted to be distinguishable were retained on the final list as candidates to identify amino acid variants of plasma proteins synthesized by hepatocytes.

Exemplary peptide pairs/triplets are shown in Table 1.

TABLE 1
Candidates for identifying amino acid variants of hepatocyte synthesized proteins

| Protein | Peptide | SEQ ID NO: |
|---|---|---|
| albumin | QTALVELVK | 15 |
| albumin | QTALVELMK | 16 |
| albumin | AVMDDFAAFVEK | 17 |
| albumin | AVMDAFAAFVEK | 18 |
| albumin | AVMDGFAAFVEK | 19 |
| Serotransferrin | SDNCEDTPEAGYFAIAVVK | 20 |
| Serotransferrin | SDNCEDTPEAGYFAVAVVK | 21 |
| Serotransferrin | PVEEYANCHLAR | 22 |
| Serotransferrin | SVEEYANCHLAR | 23 |
| Hemopexin | WDRELISER | 24 |
| Hemopexin | WDWELISER | 25 |
| Inter-alpha-trypsin inhibitor | VVPDSTPSWANPSPTPVISMLAQGSQVLESTPPPHVMR | 26 |
| heavy chain H2 | | |
| Inter-alpha-trypsin inhibitor | VVPDSTPSWANPSATPVISMLAQGSQVLESTPPPHVMR | 27 |
| heavy chain H2 | | |
| Alpha-1-antitrypsin | ADTHDEILEGLNFNLTEIPEAQIHEGFQELLRTLNQPDSQLQL | 28 |
| | TTGNGLFLSEGLK | |
| Alpha-1-antitrypsin | ADTHDEILEGLNFNLTEIPEAQIHEGFQELLHTLNQPDSQLQL | 29 |
| | TTGNGLFLSEGLK | |
| Alpha-1-antitrypsin | DTEEEDFHVDQVTTVK | 30 |
| Alpha-1-antitrypsin | DTEEEDFHVDQATTVK | 31 |
| Alpha-1-antitrypsin | PFVFLMIEQNTK | 32 |
| Alpha-1-antitrypsin | PFVFLMIDQNTK | 33 |
| Fibrinogen beta chain | GSWYSMR | 34 |
| Fibrinogen beta chain | GSWYSMK | 35 |
| Fibrinogen gamma chain | YLQEIYNSNNQK | 36 |
| Fibrinogen gamma chain | YLQEIHNSNNQK | 37 |
| Haptoglobin | VTSIQDWVQK | 38 |
| Haptoglobin | VTSIQHWVQK | 39 |
| Fibrinogen alpha chain | PGSSGPGSTGSWNSGSSGTGSTGNQNPGSPR | 40 |
| Fibrinogen alpha chain | PGSSGPGSAGSWNSGSSGTGSTGNQNPGSPR | 41 |
| Vitamin D-binding protein | LPDATPTELAK | 3 |
| Vitamin D-binding protein | LPAATPTELAK | 42 |
| Alpha-1-acid glycoprotein 2 | QNQCFYNSSYLNVQR | 43 |
| Alpha-1-acid glycoprotein 2 | QNQCFYNSSYLNAQR | 44 |
| Inter-alpha-trypsin inhibitor | LAILPASAPPATSNPDPAVSR | 45 |
| heavy chain H4 | | |
| Inter-alpha-trypsin inhibitor | LAILPASATPATSNPDPAVSR | 46 |
| heavy chain H4 | | |
| Transthyretin | LLLLCLAGLVFVSEAGPTGTGESK | 47 |
| Transthyretin | LLLLCLAGLVFVSEAGPTGTSESK | 48 |
| Inter-alpha-trypsin inhibitor | ANLSSQALQMSLDYGFVTPLTSMSIR | 49 |
| heavy chain H1 | | |
| Inter-alpha-trypsin inhibitor | ANLSSQALRMSLDYGFVTPLTSMSIR | 50 |
| heavy chain H1 | | |
| Alpha-1B-glycoprotein | PLANVTLTCQAHLETPDFQLFK | 51 |
| Alpha-1B-glycoprotein | PLANVTLTCQARLETPDFQLFK | 52 |

### Example 2. Identification and Characterization of Candidate Peptides

The purpose of the experiment was to identify and characterize peptides of isoforms of plasma proteins that can be identified by mass spectroscopy. This Example shows a bottom-up computational method for candidate peptide identification. Desirable characteristics of candidate peptides include the following:
population prevalence of 5% or greater for each variant allele;
peptides from different isoforms should have unique sequences;
peptides should be stable to chromatography methods;
peptides with greater than four fragment ions;
peptides without glutamine (Q), glutamic acid (E) or proline (P) at its N-terminus (“Q N-term,” “E N-term,” or “P N-term”);
peptides without methionine (M) or asparagine (N) residues; and
missed cleavages of peptides should be avoided (e.g., cryptic tryptic cleavage sites where the enzyme did not cleave at a cleavage site (K, R)).

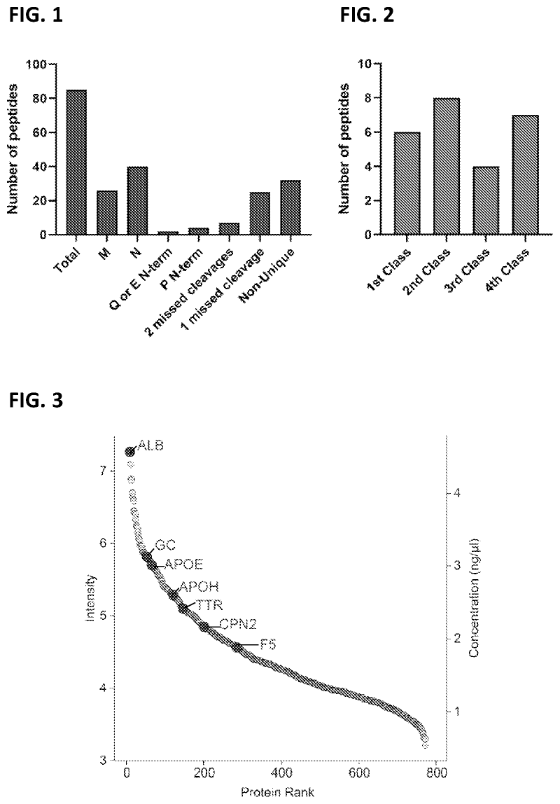

Over 1000 proteomes were screened including longitudinal datasets from previously measured plasma samples. These individuals were analyzed using data dependent acquisition (DDA) to generate MS1 and MS2 spectra of peptides present in each samples. Typically, a reference database (FASTA file) is used to identify the sequences of all human proteins with a database search. However, this reference FASTA file usually consists only of one allele variant, typically the most frequent variant in a population. Hence, it does not take variant peptides into account. To identify the variant peptides using a database search, a FASTA file containing sequences of all human proteins and their allele variants was bioinformatically designed. From this analysis, 85 candidate variant peptides were identified and screened for desirable characteristics (FIG. 1). These criteria included that the peptide is reproducible identified in longitudinal datasets, has stable chromatographic retention time, the sequence is unique, none of the amino acids Q, E, P should be at the N-terminus, the peptide should not include a methionine and no missed cleavage of the tryptic enzyme. The candidates were then grouped into classes as follows: First class-meets all criteria; Second class-misses on one criterion, maintains especially preferred criteria of reproducible identification over time in the longitudinal dataset; Third class-misses on one criterion which may be a preferred criterion; Fourth class-misses on two criteria (FIG. 2). Peptides in the first and second classes were selected for further characterization (Table 2).

TABLE 2
Candidate peptides and characteristics

| | Sequence/ | Uni- | | Q | E | | P | Missed | |
|---|---|---|---|---|---|---|---|---|---|
| Gene names | SEQ ID | que | M | N-term | N-term | N | N-term | cleavage | Frequency |
| F5 | LLSLGAGEFK | | | | | | | | 91 |
| | (SEQ ID NO: 1) | | | | | | | | |
| F5 | LLSLGAGEFR | | | | | | | | 6 |
| | (SEQ ID NO: 2) | | | | | | | | |
| GC | LPDATPTELAK | | | | | | | | 21 |
| | (SEQ ID NO: 3) | | | | | | | | |
| GC | LPEATPTELAK | | | | | | | | 85 |
| | (SEQ ID NO: 4) | | | | | | | | |
| TTR; HEL111 | AADDTWEPFSSGK | | | | | | | | 4 |
| | (SEQ ID NO: 5) | | | | | | | | |
| TTR; HEL111 | AADDTWEPFASVK | | | | | | | | 8 |
| | (SEQ ID NO: 6) | | | | | | | | |
| APOE3/2 | LGADMEDVCGR | X | X | | | | | | 7 |
| | (SEQ ID NO: 7) | | | | | | | | |
| APOE4 | LGADMEDVR | | X | | | | | | 37 |
| | (SEQ ID NO: 8) | | | | | | | | |
| APOE3/4 | LAVYQAGAR | | | | | | | | 78 |
| | (SEQ ID NO: 9) | | | | | | | | |
| APOE2 | CLAVYQAGAR | | | | | | | | 8 |
| | (SEQ ID NO: 10) | | | | | | | | |
| APOH | EHSSLAFWK | | | | X | | | | 97 |
| | (SEQ ID NO: 11) | | | | | | | | |
| APOH | EHSSLAFSK | | | | X | | | | 17 |
| | (SEQ ID NO: 12) | | | | | | | | |
| CPN2 | LTVSIEAR | | | | | | | | 73 |
| | (SEQ ID NO: 13) | | | | | | | | |
| CPN2 | LTMSIEAR | | X | | | | | | 5 |
| | (SEQ ID NO: 14) | | | | | | | | |

The peptides were all derived from proteins expressed in the liver. For this purpose the organ-specific expression of these proteins was confirmed by the Human Protein Atlas.

### Analysis of Peptides

Heavy isotope standards of all fourteen experiments were designed and analyzed by mass spectrometry. The heavy isotope standards should behave the same from the retention time and will only have a distinct mass difference due to the heavy isotope. All fourteen peptides in Table 2 could be analyzed by mass spectrometry (triplicate experiments; Table 3). Collision cross section (CCS), retention time and mass spectra were acquired. This characterization of the heavy isotope standards allows us to use them as defined standard that can be added to a sample, allowing absolute quantification and a much higher likelihood to identify the endogenous peptide in the plasma sample.

TABLE 3

| Gene | | | | |
|---|---|---|---|---|
| names | Sequence | Exp. 1 | Exp. 2 | Exp. 3 |
| F5 | LLSLGAGEFK | X | X | X |
| | (SEQ ID NO: 1) | | | |
| F5 | LLSLGAGEFR | X | X | X |
| | (SEQ ID NO: 2) | | | |
| GC | LPDATPTELAK | X | X | X |
| | (SEQ ID NO: 3) | | | |
| GC | LPEATPTELAK | X | X | X |
| | (SEQ ID NO: 4) | | | |
| TTR; | AADDTWEPFSSGK | X | X | X |
| HEL111 | (SEQ ID NO: 5) | | | |
| TTR; | AADDTWEPFASVK | X | X | X |
| HEL111 | (SEQ ID NO: 6) | | | |
| APOE3/2 | LGADMEDVCGR | X | X | X |
| | (SEQ ID NO: 7) | | | |
| APOE4 | LGADMEDVR | X | X | X |
| | (SEQ ID NO: 8) | | | |
| APOE3/4 | LAVYQAGAR | X | X | X |
| | (SEQ ID NO: 9) | | | |
| APOE2 | CLAVYQAGAR | — | — | X |
| | (SEQ ID NO: 10) | | | |
| APOH | EHSSLAFWK | X | X | X |
| | (SEQ ID NO: 11) | | | |
| APOH | EHSSLAFSK | X | X | X |
| | (SEQ ID NO: 12) | | | |
| CPN2 | LTVSIEAR | X | X | X |
| | (SEQ ID NO: 13) | | | |
| CPN2 | LTMSIEAR | X | X | X |
| | (SEQ ID NO: 14) | | | |

The heavy isotope standards of the peptides should match approximately the concentration of the endogenous peptides. To determine this concentration for the standards, the protein concentration was calculated by taking the average protein intensity of the above mentioned dataset of more than 1000 proteomes. The concentration of the highest abundant protein (albumin) is well described. As the quantitative protein levels of albumin and the six proteins (GC, APOE, APOH, TTR, CPN2, F5) for which variant peptides were identified were available, their concentration was estimated (FIG. 3).

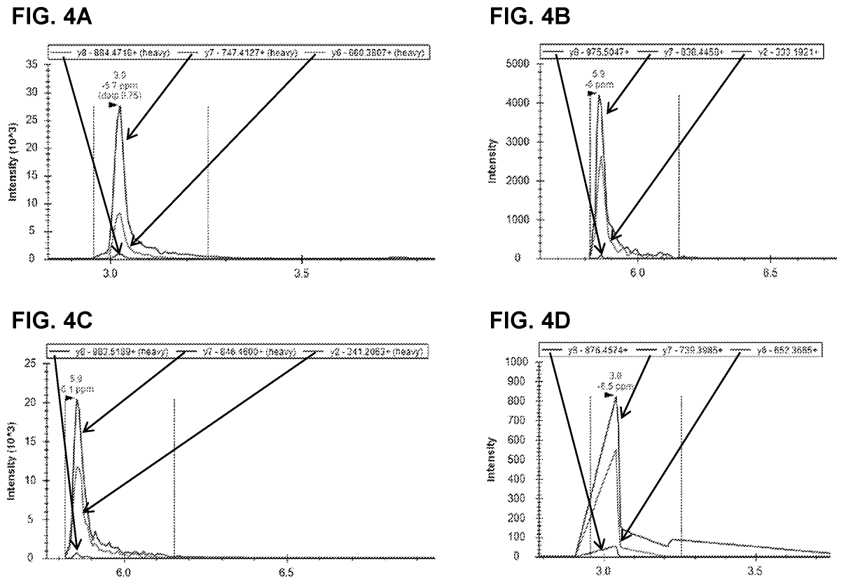

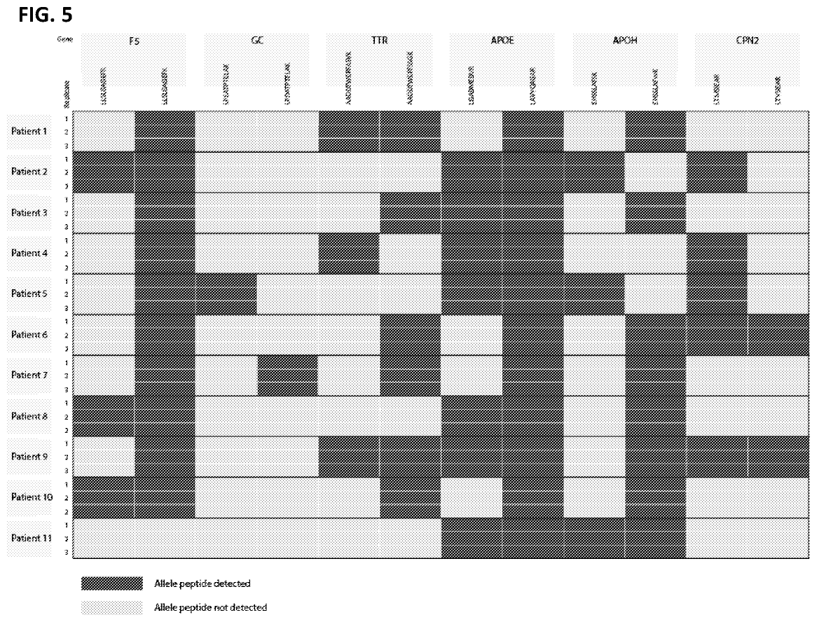

To detect and quantify peptides indicative of the allele variants in plasma samples, a targeted method was designed (Parallel-reaction-monitoring-PRM) specifically identifying and quantifying these peptides. Moreover, heavy isotope labeled peptide standards of the selected peptide candidates for six different proteins were designed, consisting of a total of twelve variant peptides. FIG. 4 shows exemplified spectra of the y8, y7 and y2 or the y8, y7 and y6 peptide fragment ions (heavy standards and endogenous light peptides) from two different selected APOH allele variants.

Spike-in experiments of the heavy isotope standards into real samples were performed to determine the level of resolution of the targeted assay. To illustrate the potential to detect variant peptides specific to an allele, we then set out to target the peptides across eleven different individuals in triplicate experiments (FIG. 5). By analyzing the presence of these 12 allele variants, it was possible to distinguish the 11 individuals.

In some experiments, the tryptic digestion is performed after the samples are spiked in, in other experiments, the tryptic digestion is performed before the samples are spiked in.

These data demonstrate that candidate peptides identified herein are indeed detectable by MS, and can distinguish between individuals.

### Example 3. Simulation of Quantitative Alteration of Variant Peptides in the Transplantation Setting

### Introduction

Mass spectrometry (MS)-based proteomics can be used to identify and quantify variant peptides in plasma samples. The quantitative aspect is important as it allows tracking, for example in liver transplantation, the increase or decrease of variant peptides that are different between donor and recipient. This allows the monitoring of the status of the transplanted organ. This Example shows the simulation of quantitative alteration of variant peptides in the transplantation setting, using apolipoprotein E (APOE) as an exemplary protein, which is produced by the liver and secreted into the circulation. As shown in Example 2, the tryptic peptide LGADMEDVR (SEQ ID NO:8) is specific for carriers of the APOE4 allele, whereas the peptide LAVYQAGAR (SEQ ID NO:9) is present in APOE3 and APOE4 carriers. This Example shows that different polypeptide isoforms can be detected from the same sample, and can each be quantified.

### Methods and Results

Plasma samples containing both allele peptides were prepared for bottom-up MS-based proteomics to produce the tryptic peptides for allele monitoring. Briefly, the samples were heat denatured, disulfide bonds were reduced and cysteine residues were alkylated. Trypsin was used to digest proteins into peptides. Next, both heavy isotope-labeled variant peptides (corresponding to both alleles) were added in different amounts to the digested plasma samples to mimic the increase of a variant peptide in plasma after liver transplantation. Samples were then measured using a combination of a liquid chromatography (LC) system coupled to a mass spectrometer. An Evosep LC system was used, which requires the samples to be loaded on C-18 material tips to act as both a desalting material and a precolumn. Data acquisition was performed with a data-dependent acquisition method. Interpretation of raw data was performed with the software AlphaPept.

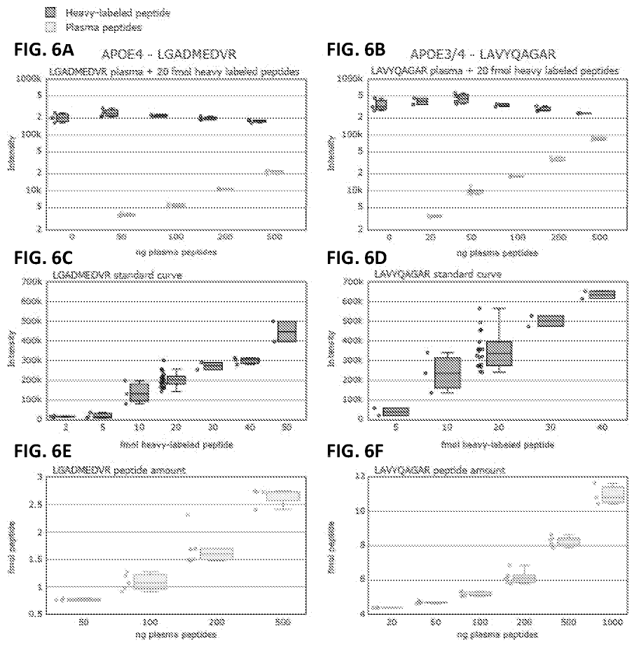

The dilution of the plasma peptides into the heavy isotope-labeled variant should mimic the increase of a variant peptide in plasma after liver transplantation. In this experiment all peptide species (both allele peptides, heavy isotope-labeled and unlabeled) were present in all measurements and quantified in parallel. This experimental design prevented a mix-up of alleles and allowed a clear dilution curve for the monitored peptides. This analysis demonstrated that increasing amounts of the plasma variant peptides can be detected in a dilution series with a constant amount of heavy isotope-labeled peptide (FIGS. 6A-6B). The analyzed protein amounts ranged from 20 ng to 1 μg, corresponding to 0.4 nl to 20 nl of plasma input material. The intensity for this untargeted screening experiment ranged from 3k to 700k, spanning over three orders of magnitude, that can be further extended using targeted assay strategies. Using a standard curve with known concentrations of the heavy isotope-labeled peptides (FIGS. 6C-6D), absolute quantification of the ApoE variant peptides was possible (FIGS. 6E-6F). The quantified peptide amounts ranged from 0.7 fmol to 12 fmol of the ApoE variant peptides in 20 ng to 1000 ng of plasma peptides.

### Example 4. Identification and Characterization of Candidate Peptides for Identifying Amino Acid Variants of B Cell Specific Proteins

### Introduction

The purpose of the experiment was to identify amino acid variants of B cell specific proteins, and peptides of isoforms of B cell specific proteins that can be identified by mass spectroscopy. The criteria for selecting candidate B cell proteins was similar as described in Example 1. Desirable characteristics of candidate peptides were similar as described in Example 2.

### Methods and Results

Similar steps as described in Example 1 were used to identify amino acid variants of B cell specific proteins here. Briefly:

A) Selecting abundant B-cell specific proteins: Lists of proteins found in plasma are available in the public domain (e.g., Anderson Mol Cell Proteomics 2002), from which a list of B cell-specific proteins were identified, and ordered by their respective concentrations in plasma. 6 immunoglobulin proteins were identified as specific to B cells and plasma cells using the Human Protein Atlas (https://www.proteinatlas.org/), namely, immunoglobulin heavy constant gamma 1 (IGHG1), immunoglobulin heavy constant gamma 2 (IGHG2), immunoglobulin heavy constant alpha 1 (IGHA1), immunoglobulin heavy constant mu (IGHM), immunoglobulin kappa constant (IGKC), and immunoglobulin lambda constant (IGLC2). A list of genes identified by searching cell type term on Human Protein Atlas were downloaded as a spreadsheet, and sorted by Single Cell Type RNA abundance.

B) Mapping to corresponding genes: Top 6 proteins from the above identified list (the 6 immunoglobulin proteins) were mapped to corresponding genes using genomeADbrowser (https://gnomad.broadinstitute.org/).

C) Identification of prevalent missense mutation: Missense mutations in the genes mapped in step B) were identified using genomeADbrowser, then ordered from high to low by frequency of missense mutations. Genes with a population mutation frequency of greater than 5% were selected for further analysis.

D) Identification of prevalent amino acid variants: Genes with a population mutation frequency of greater than 5% identified in step C) were further searched in the NCBI dbSNP database (https://www.ncbi.nlm.nih.gov/snp/) to obtain variant population frequency, and determine which allele is associated with which polypeptide isoform. Protein sequences of amino acid variants with population frequency above 5% (IGHG1, IGHG2, IGHA1, IGHM, and IGKC) were downloaded from the UniProt database (https://www.uniprot.org/) or genomeADbrowser.

E) Identification of tryptic peptides: Prevalent amino acid variants identified from the above steps were digested with trypsin in silico. Protein Prospector (http://prospector.ucsf.edu/prospector/mshome.htm) was used to identify tryptic peptides that are distinguishable, resulting in a final list of candidates to identify amino acid variants of B cell specific proteins.

Exemplary peptide pairs are shown in Table 4.

TABLE 4
Candidates for identifying amino acid variants of B cell specific proteins

| | | SEQ |
|---|---|---|
| | | ID |
| Protein | Peptide | NO: |
| IGHG1 | DELTKNQVSLTCLVK | 53 |
| IGHG1 | EELTKNQVSLTCLVK | 54 |
| IGHG1 | DELTKNQVSLTCLVK | 55 |
| IGHG1 | DEMTKNQVSLTCLVK | 56 |
| IGHG1 | WQQGNVFSCSVMHEALHNHYTQK | 57 |
| IGHG1 | WQQGNVFSCSVMHEGLHNHYTQK | 58 |
| IGHG2 | GFYPSDISVEWESNGQPENNYK | 59 |
| IGHG2 | GFYPADISVEWESNGQPENNYK | 60 |
| IGHG2 | DYFPEPVTVSWNSGALTSGVHTFPAVLQSSGLYSLSSVVTVPSSNFGTQTYTCNVDHK | 61 |
| IGHG2 | DYFPEPVTVSWNSGALTSGVHTFPAVLQSSGLYSLSSVVTVTSSNFGTQTYTCNVDHK | 62 |
| IGHG2 | TPEVTCVVVDVSHEDPEVQFNWYVDGVEVHNAK | 63 |
| IGHG2 | TPEVTCVVVDVSHEDPEVQFNWYVDGMEVHNAK | 64 |
| IGHA1 | SAVQGPPER | 65 |
| IGHA1 | SAVQGPPDR | 66 |
| IGHM | GLTFQQNASSMCVPDQDTAIR | 67 |
| IGHM | GLTFQQNASSMCGPDQDTAIR | 68 |
| IGHM | ESDWLGQSMFTCR | 69 |
| IGHM | ESDWLSQSMFTCR | 70 |
| IGKC | VYACEVTHQGLSSPVTKSFNR | 71 |
| IGKC | LYACEVTHQGLSSPVTKSFNR | 72 |

1. (canceled)

2. A method for detecting a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% of the isoforms of the polypeptide in a population of individuals; and wherein detection of donor-specific isoforms of the one or more polypeptides indicates the presence of the donor cell in the recipient.

3. (canceled)

4. A method for quantitating a donor cell in a recipient, the method comprising detecting specific isoforms of one or more polypeptides in a sample from the recipient; wherein the donor cell and the recipient express different specific isoforms of each of the one or more polypeptides, wherein the specific isoforms comprise greater than about 1% of the isoforms of the polypeptide in a population of individuals; and wherein the ratio of donor-specific isoforms to recipient-specific isoforms of the one or more polypeptides indicates a quantitative measure of the donor cell compared to the recipient cell or a measure of the performance of the donor cell in the recipient.

5-8. (canceled)

9. A method for monitoring engraftment and/or function of a donor cell in a recipient, the method comprising detecting specific isoforms of one or more polypeptides in samples from the recipient; wherein the samples have been obtained from the recipient one or more times after administration of the donor cell to the recipient; wherein the donor cell and the recipient express different specific isoforms of each of the one or more polypeptides, and wherein the specific isoforms comprise greater than about 1% of the isoforms of the polypeptide in a population of individuals;
wherein detection of donor-specific isoforms of the one or more polypeptides indicates engraftment and/or function of the donor cell in the recipient.

10. The method of claim 2, wherein the sample is isolated or obtained from the recipient within at least about one week after administration of the donor cell.

11. The method of claim 2, wherein the sample is a plasma sample, a serum sample, a whole blood sample, a lymph sample, a cerebrospinal fluid sample, a urine sample, a liquid biopsy sample, or a solid biopsy sample.

12-13. (canceled)

14. The method of claim 2, wherein the donor cell is a mesenchymal stromal stem cell, or a stem cell derived from a tissue.

15-19. (canceled)

20. The method of claim 2,
(i) wherein the donor cell is a hepatocyte, a stem cell, or a B cell; and/or
(ii) wherein the donor cell is genetically engineered.

21-25. (canceled)

26. The method of claim 2, wherein the one or more polypeptides are organ-specific or tissue-specific polypeptides.

27-32. (canceled)

33. The method of claim 2, wherein the one or more polypeptides are enzymatically cleaved prior to analysis to generate a peptide mixture.

34. (canceled)

35. The method of claim 33, wherein the one or more polypeptides are cleaved with trypsin prior to analysis to generate the peptide mixture.

36-38. (canceled)

39. The method of claim 2, wherein the specific isoforms are detected by tandem mass spectrometry.

40-46. (canceled)

47. A method for monitoring engraftment of a donor liver cell in a recipient, the method comprising:
a) isolating samples from the recipient after administration of the donor liver cell to the recipient; and
b) detecting donor-specific isoforms of one or more polypeptides in the samples using the method of claim 2;
wherein detection of donor-specific isoforms of the one or more polypeptides indicates engraftment of the donor liver cell in the recipient.

48-53. (canceled)

54. The method of claim 47, wherein the one or more polypeptides comprise one or more of alpha-1-antitrypsin (AAT), vitamin D binding protein (GC), coagulation factor V (F5), transthyretin (TTR), apolipoprotein E (APOE), beta-2-glycoprotein 1 (APOH), and carboxypeptidase N subunit 2 (CPN2).

55-57. (canceled)

58. The method of claim 54,
(i) wherein the one or more polypeptides comprise AAT, and wherein the specific isoforms comprise the amino acid sequence of SEQ ID NO:30 and/or SEQ ID NO:31;
(ii) wherein the one or more polypeptides comprise F5, and wherein the specific isoforms comprise the amino acid sequence of SEQ ID NO:1 and/or SEQ ID NO:2;
(iii) wherein the one or more polypeptides comprise GC, and wherein the specific isoforms comprise the amino acid sequence of SEQ ID NO:3, SEQ ID NO:4, and/or SEQ ID NO: 42;
(iv) wherein the one or more polypeptides comprise TTR, and wherein the specific isoforms comprise the amino acid sequence of SEQ ID NO:5 and/or SEQ ID NO:6;
(v) the one or more polypeptides comprise APOE, and wherein the specific isoforms comprise the amino acid sequence of SEQ ID NO:7 and/or SEQ ID NO: 8;
(vi) wherein the one or more polypeptides comprise APOE, and wherein the specific isoforms comprise the amino acid sequence of SEQ ID NO:9 and/or SEQ ID NO:10;
(vii) wherein the one or more polypeptides comprise APOH, and wherein the specific isoforms comprise the amino acid sequence of SEQ ID NO:11 and/or SEQ ID NO:12; and/or
(viii) wherein the one or more polypeptides comprise CPN2, and wherein the specific isoforms comprise the amino acid sequence of SEQ ID NO: 13 and/or SEQ ID NO:14.

59-81. (canceled)

82. A method for treating a liver disorder in an individual in need thereof, the method comprising:
a) administering donor liver cells from a donor individual to the recipient individual in need thereof;
b) isolating samples from the recipient individual after administration of the donor liver cells; and
c) detecting donor-specific isoforms of one or more polypeptides in the samples using the method of claim 2;
wherein detection of donor-specific isoforms of the one or more polypeptides indicates engraftment of the donor liver cells in the recipient individual in need thereof.

83-89. (canceled)

90. A method for monitoring engraftment of a donor B cell in a recipient, the method comprising:
a) isolating samples from the recipient after administration of the donor B cell to the recipient; and
b) detecting donor-specific isoforms of one or more polypeptides in the samples using the method of claim 2;
wherein detection of donor-specific isoforms of the one or more polypeptides indicates engraftment of the donor B cell in the recipient.

91-107. (canceled)

108. The method of claim 90, wherein the one or more polypeptides comprise one or more of immunoglobulin heavy constant gamma 1 (IGHG1), immunoglobulin heavy constant gamma 2 (IGHG2), immunoglobulin heavy constant alpha 1 (IGHA1), immunoglobulin heavy constant mu (IGHM), and immunoglobulin kappa constant (IGKC).

109-111. (canceled)

112. The method of claim 108,
(i) wherein the one or more polypeptides comprise IGHG1, and wherein the specific isoforms comprise the amino acid sequence of a) one or more of SEQ ID NOs: 53-56, or b) SEQ ID NO:57 and/or SEQ ID NO:58;
(ii) wherein the one or more polypeptides comprise IGHG2, and wherein the specific isoforms comprise the amino acid sequence of a) SEQ ID NO:59 and/or SEQ ID NO:60, b) SEQ ID NO: 61 and/or SEQ ID NO:62, or c) SEQ ID NO:63 and/or SEQ ID NO:64;
(iii) wherein the one or more polypeptides comprise IGHA1, and wherein the specific isoforms comprise the amino acid sequence of SEQ ID NO:65 and/or SEQ ID NO:66;
(iv) wherein the one or more polypeptides comprise IGHM, and wherein the specific isoforms comprise the amino acid sequence of a) SEQ ID NO:67 and/or SEQ ID NO:68, or b) SEQ ID NO:69 and/or SEQ ID NO:70; and/or
(v) wherein the one or more polypeptides comprise IGKC, and wherein the specific isoforms comprise the amino acid sequence of SEQ ID NO:71 and/or SEQ ID NO:72.

113-119. (canceled)

120. A method for treating a B cell disorder in an individual in need thereof, the method comprising:
a) administering donor B cells from a donor individual to the recipient individual in need thereof;
b) isolating samples from the recipient individual after administration of the donor B cells; and
c) detecting donor-specific isoforms of one or more polypeptides in the samples using the method of claim 2;
wherein detection of donor-specific isoforms of the one or more polypeptides indicates engraftment of the donor B cells in the recipient individual in need thereof.

121-127. (canceled)

128. A method for treating an individual in need thereof, the method comprising:
a) administering donor cells from a donor individual to the recipient individual in need thereof;
b) isolating samples from the recipient individual after administration of the donor cells; and
c) detecting specific isoforms of one or more polypeptides in the samples; wherein the donor cells and cells from the recipient individual express different specific isoforms of each of the one or more polypeptides, and wherein the specific isoforms comprise greater than about 1% of the isoforms of the polypeptide in a population of individuals;
wherein detection of donor-specific isoforms of the one or more polypeptides indicates engraftment of donor cells in the recipient individual, thereby treating the individual.

129-132. (canceled)
